SEQUENCE LISTING (14 sequences)

SEQ ID NO: 1 - protein, 218 aa, Artificial Sequence
  Description of Artificial Sequence Synthetic polypeptide
DIVMTQSPDSLAVSLGERATINCKASQSVDYDGDSYMNWYQQKPGQPPKLLIYVASNLESGVPDRFSGSGSGTDFTLTISSLQAEDVAVYYCQQSLQDPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 218 aa, Artificial Sequence
  Description of Artificial Sequence Synthetic polypeptide
DIVMTQSPDSLAVSLGERATINCKASQSVDYDGDSYMNWYQQKPGQPPKLLIYVASNLESGVPDRFSGSGSGTDFTLTISSLQAEDVAVYYCQQSLQDPPTFGGGTKVEIKRTVAALSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 447 aa, Artificial Sequence
  Description of Artificial Sequence Synthetic polypeptide
QVQLVQSGAEVKKPGASVKVSCKASGYTFTAYVISWVRQAPGQGLEWMGEIYPGSGSSYYNEKFKGRVTMTRDTSTSTVYMELSSLRSEDTAVYYCARSGDGSRFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 447 aa, Artificial Sequence
  Description of Artificial Sequence Synthetic polypeptide
QVQLVQSGAEVKKPGASVKVSCKASGYTFTAYVISWVRQAPGQGLEWMGEIYPGSGSSYYNEKFKGRVTMTRDTSTSTVYMELSSLRSEDTAVYYCARSGDGSRFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 447 aa, Artificial Sequence
  Description of Artificial Sequence Synthetic polypeptide
QVQLVQSGAEVKKPGASVKVSCKASGYTFTAYVISWVRQAPGQGLEWMGEIYPGSGSSYYNEKFKGRVTMTRDTSTSTVYMELSSLRSEDTAVYYCARSGDGSRFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 6 - protein, 447 aa, Artificial Sequence
  Description of Artificial Sequence Synthetic polypeptide
QVQLVQSGAEVKKPGASVKVSCKASGYTFTAYVISWVRQAPGQGLEWMGEIYPGSGSSYYNEKFKGRVTMTRDTSTSTVYMELSSLRSEDTAVYYCARSGDGSRFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHHHYTQKSLSLSPG

SEQ ID NO: 7 - protein, 15 aa, Artificial Sequence
  Description of Artificial Sequence Synthetic peptide
KASQSVDYDGDSYMN

SEQ ID NO: 8 - protein, 7 aa, Artificial Sequence
  Description of Artificial Sequence Synthetic peptide
VASNLES

SEQ ID NO: 9 - protein, 9 aa, Artificial Sequence
  Description of Artificial Sequence Synthetic peptide
QQSLQDPPT

SEQ ID NO: 10 - protein, 10 aa, Artificial Sequence
  Description of Artificial Sequence Synthetic peptide
GYTFTAYVIS

SEQ ID NO: 11 - protein, 18 aa, Artificial Sequence
  Description of Artificial Sequence Synthetic peptide
GEIYPGSGSSYYNEKFKG

SEQ ID NO: 12 - protein, 8 aa, Artificial Sequence
  Description of Artificial Sequence Synthetic peptide
GDGSRFVY

SEQ ID NO: 13 - protein, 280 aa, Artificial Sequence
  Description of Artificial Sequence Synthetic polypeptide
AESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSHHHHHH

SEQ ID NO: 14 - protein, 285 aa, Artificial Sequence
  Description of Artificial Sequence Synthetic polypeptide
AESHLSLLYHLTAVSSPAPGTPAFWVSGWLGPQQYLSYDSLRGQAEPCGAWVWENQVSWYWEKETTDLRIKEKLFLEAFKALGGKGPYTLQGLLGCELSPDNTSVPTAKFALNGEEFMNFDLKQGTWGGDWPEALAISQRWQQQDKAANKELTFLLFSCPHRLREHLERGRGNLEWKEPPSMRLKARPGNPGFSVLTCSAFSFYPPELQLRFLRNGLAAGTGQGDFGPNSDGSFHASSSLTVKSGDEHHYCCIVQHAGLAQPLRVELESPAKSSGRAHHHHHHHH

(19) United States
(12) Patent Application Publication
(10) Pub. No.: US 2010/0021460 A1
(43) Pub. Date: Jan. 28, 2010

(54) Methods of Treating Autoimmune Diseases Using CD4 Antibodies

(75) Inventors: Rong Deng, Pleasanton, CA (US); Paul J. Fielder, Emerald Hills, CA (US); Henry B. Lowman, El Granada, CA (US); Eric Stefanich, Redwood City, CA (US); Yanan Zheng, Redwood City, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 12/502,953

(22) Filed: Jul. 14, 2009

Related U.S. Application Data
(60) Provisional application No. 61/081,012, filed on Jul. 15, 2008.

(51) Int. Cl. A61K 39/395; A61P 37/00

(52) U.S. Cl. 424/133.1

(57) ABSTRACT

Methods of treating autoimmune disorders in mammalian subjects using non-depleting CD4 antibodies, alone or in combination with other compounds, are provided.

### CROSS REFERENCE TO RELATED APPLICATION

This application claims the benefit of priority of provisional U.S. Application No. 61/081,012 filed Jul. 15, 2008, which is hereby incorporated by reference in its entirety.

### FIELD

Methods of treating autoimmune disorders in mammalian subjects using non-depleting anti-CD4 antibodies, alone or in combination with other compounds, are provided.

### BACKGROUND

Autoimmune diseases, such as lupus, myasthenia gravis, multiple sclerosis (MS), rheumatoid arthritis (RA), psoriasis, inflammatory bowel disease, asthma and idiopathic thrombocytopenic purpura, among others, remain clinically important diseases in humans.

Lupus is an autoimmune disease involving antibodies that attack connective tissue. The disease is estimated to affect nearly 1 million Americans, primarily women between the ages of 20-40. Various forms of lupus are known, including, but not limited to, systemic lupus erythematosus (SLE), cutaneous lupus erythematosus (CLE), lupus nephritis (LN). Untreated lupus can be fatal as it progresses from attack of skin and joints to internal organs, including lung, heart, and kidneys (with renal disease being the primary concern).

Currently, there are no curative treatments for patients who have been diagnosed with SLE. Typically, patients are treated with any of a number of powerful immunosuppressive drugs such as high-dose corticosteroids, e.g., prednisone, or azathioprine or cyclophosphamide. Many of these drugs have potentially harmful side effects to the patients being treated. In addition, these immunosuppressive drugs interfere with the person's ability to produce all antibodies, not just the self-reactive anti-DNA antibodies. Immunosuppressants also weaken the body's defense against other potential pathogens, thereby making the patient extremely susceptible to infection and other potentially fatal diseases, such as cancer. In some of these instances, the side effects of current treatment modalities, combined with continued low-level manifestation of the disease, can cause serious impairment and premature death.

Certain recent therapeutic regimens include cyclophosphamide, mycophenolate mofetil (MMF), methotrexate, antimalarials, hormonal treatment (e.g., DHEA), and anti-hormonal therapy (e.g., the anti-prolactin agent bromocriptine). Methods for treatment of SLE involving anti-DNA antibodies have also been described. (U.S. Pat. No. 4,690,905; U.S. Pat. No. 6,726,909).

High-dose intravenous immune globulin (IVIG) infusions have also been used in treating certain autoimmune diseases. Up until the present time, treatment of SLE with IVIG has provided mixed results, including both resolution of lupus nephritis (Akashi et al., J. Rheumatology 17:375-379 (1990)), and in a few instances, exacerbation of proteinuria and kidney damage (Jordan et al., Clin. Immunol. Immunopathol. 53: S164-169 (1989)).

Multiple Sclerosis (MS) is a disorder of the central nervous system that affects the brain and spinal cord. Current treatments for MS include corticosteroids, beta interferons (BETAFERON®, AVONEX®, REBIF®), glatiramer acetate (COPAXONE®), methotrexate, azathioprine, cyclophosphamide, cladribine, baclofen, tizanidine, amitriptyline, carbamazepine (Berkow et al. (ed.), 1999, supra) and natalizumab (TYSABRI®).

Rheumatoid arthritis (RA) is a chronic systemic autoimmune inflammatory disease affecting between 1.3 and 2.1 million persons in the United States. Various drugs have been used to treat RA symptoms. None of the treatments clearly stop progression of joint destruction (Harris E D. Rheumatoid Arthritis: The clinical spectrum. In Textbook of Rheumatology [Kelley, et al., eds.] W B Saunders, Philadelphia pp 915-990, 1985).

RA is an autoimmune disorder of unknown etiology. Most RA patients suffer a chronic course of disease that, even with currently available therapies, may result in progressive joint destruction, deformity, disability and even premature death. More than 9 million physician visits and more than 250,000 hospitalizations per year result from RA. The goals of RA therapy are to prevent or control joint damage, prevent loss of function and decrease pain.

Because the body produces tumor necrosis factor alpha (TNFα) during RA, TNFα inhibitors have been used for therapy of RA. Exemplary TNFα inhibitors include etanercept (sold under the trade name ENBREL®), infliximab (sold under the trade name REMICADE®), adalimumab (sold under the trade name HUMIRA®), golimumab (sold under the trade name SIMPONI™), and certolizumab pegol (sold under the trade name CIMZIA®).

In various instances, administration of therapeutic agents to treat RA rapidly induces adverse side effects, or events, including but not limited to fever, headache, nausea, vomiting, breathing difficulties and changes in blood pressure. Increased risk of serious and/or life-threatening infections is particularly associated with administration of TNFα inhibitors. These adverse events limit the amount of a drug or therapeutic compound that can be given, which in turn limits the therapeutic effectiveness that could be achieved with higher doses of the drug.

Despite efforts to advance RA treatment, many patients do not achieve a clinically meaningful response in terms of inflammation and joint damage. In addition, many patients in clinical practice and registries are not able to continue therapy because of intolerance of or contraindications to current therapies. At present, there are limited therapeutic alternatives for patients who have had an inadequate response to treatment, including DMARDs and/or biologic agents, representing a relatively large unmet need for these patients.

To attempt to address this unmet need, a number of groups have developed and reported on various anti-CD4 mAbs as potential therapeutic agents (reviewed in Choy et al., Br. J. Rheumatol. 37:484-490, 1998). The first anti-CD4 mAbs were murine and initial clinical studies were discontinued due to immunogenicity. (Keystone, E. C., 2003, Current Opinion in Rheum. 15:253-258). Chimeric mAbs and humanized mAbs were also developed. Administration of chimeric mAbs demonstrated no clinical efficacy and were associated with adverse events following the initial administration. (Keystone, E. C., 2003, Current Opinion in Rheum. 15:253-258). A humanized anti-CD4 monoclonal antibody administered intravenously to psoriasis and rheumatoid arthritis patients induced fever, chills, hypotension and chest tightness. (Isaacs, et al., 1997 Clin Exp Immunol, 110, 158-166). This treatment down-modulated expression of CD4 and caused a reduction in the number of circulating CD4-positive T cells resulting in severe peripheral blood CD4 lymphopenia. (Keystone, E. C., 2003, Current Opinion in Rheum. 15:253-258).

Subsequently, several non-depleting, or near-non-depleting anti-CD4 antibodies were developed and tested in clinical trials. Such non-depleting or near-non-depleting anti-CD4 antibodies include OKTcdr4a (Schulze-Koops et al., J. Rheumatol. 25:2065-76, 1998); 4162W94 (Choy et al., Rheumatol. 41:1142-1148, 2002); and clenoliximab (Idec 151). (Reddy et al., 2000, J. Immunol. 164:1925-1933; U.S. Pat. Nos. 5,756,096, 6,136,310, 7,338,658; Mould et al., Clin Pharmacol Ther 66:246-57, 1999; Hepburn et al., Rheum. 42:54-61, 2003; Luggen et al., abstract presented at the 2003 Annual European Congress of Rheumatology, Ann. Rheum. Dis. OPO110). Clinical testing of these antibodies showed at best modest therapeutic effectiveness of short duration. In addition, various undesirable side effects were observed, such as CD4 lymphopenia and skin rash. (Schulze-Koops et al., J. Rheumatol. 25:2065-76, 1998; Choy et al., Rheumatol. 41:1142-1148, 2002; Mould et al., Clin Pharmacol Ther 66:246-57, 1999; Hepburn et al., Rheum. 42:54-61, 2003; Luggen et al., abstract presented at the 2003 Annual European Congress of Rheumatology, Ann. Rheum. Dis. OPO110).

Another non-depleting anti-CD4 monoclonal antibody is TRX1 developed by ToleRx and tested in healthy human volunteers in a phase I study. (Ng et al., Pharm. Research 23:95-103, 2006). In the study, subjects received a single intravenous infusion of up to 10 mg/kg TRX1. Such administration of TRX1 was still associated with pruritic rashes. Id.

In sum, while such early-phase clinical studies of non-depleting anti-CD4 antibodies administered intravenously were encouraging in terms of certain safety parameters and possible efficacy, patients still experienced some adverse events, such as pruritic rash, and the intravenous dosing regimens required high dosages and/or frequent dosing of antibody to provide therapeutic benefit, to the extent any benefit was observed in the reported trials.

As such, those antibodies would be nonoptimal for therapeutically effective subcutaneous dosing regimens. In addition, the intravenous dosing regimens tested with those antibodies are not directly translatable into optimal subcutaneous dosing regimens. Accordingly, none of the previously reported non-depleting anti-CD4 antibodies, nor the previously reported dosing regimens, would allow for an efficient, therapeutically effective dosing regimen based on subcutaneous administration.

The present invention solves problems related to past therapies and provides additional advantages that will be apparent from the detailed description below.

### SUMMARY

The present invention provides an effective therapeutic regimen for the treatment of rheumatoid arthritis and other autoimmune diseases, including, for example, lupus, multiple sclerosis (MS), and others. The present invention also provides treatment methods that achieve therapeutic efficacy while minimizing toxicity and adverse events. Furthermore, the therapeutic molecules and treatments of this invention are relatively easy to administer, and include the capability for self-administration by the patient.

The invention provides methods of treating an autoimmune disease in a mammalian subject, e.g., a human subject. In the methods, a therapeutically effective amount of a non-depleting CD4 antibody that has been modified to increase serum half-life compared to the antibody without the modification is administered subcutaneously. Throughout the specification, the antibodies of the invention are variously referred to as “non-depleting CD4 antibody” and “non-depleting anti-CD4 antibody.” It is understood that, as used herein, these terms are synonymous and interchangeable. In one aspect, the antibody is administered subcutaneously at a dose between 0.2 mg/kg and 10 mg/kg. In another aspect, the antibody is administered subcutaneously at a dose between 0.3 mg/kg and 7.0 mg/kg. In a further aspect, the antibody is administered subcutaneously at a dose selected from 0.3 mg/kg, 1.0 mg/kg, 1.5 mg/kg, 2.0 mg/kg, 2.5 mg/kg, 3.0 mg/kg, 3.5 mg/kg, 5.0 mg/kg, and 7.0 mg/kg. In a further aspect, the antibody is administered subcutaneously at a flat dose. In certain embodiments, the flat dose is between 150 mg and 350 mg. In certain embodiments, the flat dose is between 200 mg and 300 mg. In certain embodiments, the flat dose is between 225 mg and 275 mg. In certain embodiments, the flat dose is 250 mg.

The invention also provides methods of treating an autoimmune disease in a mammalian subject, e.g., a human subject, by administering subcutaneously to the subject a first administration of a therapeutically effective amount of a non-depleting CD4 antibody that has been modified to increase serum half-life compared to the antibody without the modification, and by administering subcutaneously to the subject at least one subsequent administration of the modified non-depleting CD4 antibody. In one aspect, the first administration is at a dose between 0.05 mg/kg and 35 mg/kg and each subsequent administration is at the same dose as the first administration. In another aspect, the first administration and each subsequent administration is at a dose between 1.5 mg/kg and 5.0 mg/kg. In a further aspect, the first administration and each subsequent administration is at a dose selected from 1.5 mg/kg, 2.0 mg/kg, 3.0 mg/kg, 3.5 mg/kg, and 5.0 mg/kg. In yet another aspect, the first administration and each subsequent administration is a flat dose. In certain embodiments, the flat dose is between 150 mg and 350 mg, or between 200 mg and 300 mg, or between 225 mg and 275 mg. In certain embodiments, the flat dose is 250 mg. In another aspect, each subsequent administration is administered between five and nine days after the previous administration, or between six and eight days after the previous administration, or seven days after the previous administration. In one aspect, the non-depleting CD4 antibody is administered subcutaneously once every week. In certain such embodiments, the non-depleting CD4 antibody is administered subcutaneously once every week on a chronic basis, e.g., for at least one year, or at least two years, or at least five years, or at least ten years, or for the lifetime of the subject.

In certain embodiments, methods of treating lupus, including systemic lupus erythematosus, extrarenal/lupus nephritis, and cutaneous lupus erythematosus are provided. Methods of treating multiple sclerosis, including relapsing-remitting multiple sclerosis, secondary-progressive multiple sclerosis, and primary-progressive multiple sclerosis are also provided. The invention also provides methods of treating rheumatoid arthritis, psoriasis, and psoriatic arthritis.

In one aspect, the methods provide a modification of the antibody to increase serum half-life that increases the binding of the antibody to FcRn relative to the binding of the unmodified antibody to FcRn. In certain such embodiments, the binding of the modified antibody to FcRn is increased between 2.0-fold and 4.5-fold relative to the binding of the unmodified antibody to FcRn. In further embodiments, the binding of the modified antibody to FcRn is increased between 3.0-fold and 4.0 fold. In still further embodiments, the binding of the modified antibody to FcRn is increased between 3.3-fold and 3.7-fold. In one embodiment, the binding of the modified antibody to FcRn is increased 3.5-fold relative to the binding of the unmodified antibody to FcRn. In another aspect, the modified antibody has reduced serum clearance compared to serum clearance of the unmodified antibody. In certain embodiments, serum clearance of the modified antibody is reduced by at least 38% compared to serum clearance of the unmodified antibody. In certain embodiments, serum clearance of the modified antibody is reduced between 38% and 59% compared to serum clearance of the unmodified antibody.

In another aspect, the methods provide a non-depleting CD4 antibody containing, in addition to a modification to increase serum half-life, a further modification that reduces binding to an Fcγ receptor as compared to the antibody without the further modification. In one embodiment, the constant regions of the non-depleting CD4 antibody are derived from a human IgG1 antibody. In certain embodiments, the non-depleting CD4 antibody comprises an Fc region that is aglycosylated. In a further embodiment, the non-depleting CD4 antibody comprises a constant region that does not comprise a glycosylation site. In one aspect, the non-depleting CD4 antibody comprises an Fc region with at least one amino acid substitution. In certain such embodiments, the non-depleting antibody comprises a N297A substitution as shown in SEQ ID NOs.: 4, 5, and 6. In further embodiments, the non-depleting CD4 antibody further comprises a N434A substitution as shown in SEQ ID NO.: 5 or a N434H substitution as shown in SEQ ID NO.: 6.

In one class of embodiments, the non-depleting CD4 antibody is an anti-human CD4 antibody. In one aspect, the non-depleting CD4 antibody is humanized. In certain such embodiments, the non-depleting CD4 antibody comprises a light chain amino acid sequence set forth in SEQ ID NO.: 1; a light chain amino acid sequence set forth in SEQ ID NO.: 2; a light chain variable region amino acid sequence set forth in SEQ ID NO.: 1; a light chain variable region amino acid sequence set forth in SEQ ID NO.: 2; the light chain CDR amino acid sequences set forth in SEQ ID NO.: 1; or the light chain CDR amino acid sequences set forth in SEQ ID NO.: 2.

In one class of embodiments, the non-depleting CD4 antibody comprises a heavy chain amino acid sequence set forth in SEQ ID NO.: 5; a heavy chain amino acid sequence set forth in SEQ ID NO.: 6; a heavy chain variable region amino acid sequence set forth in SEQ ID NO.: 5; a heavy chain variable region amino acid sequence set forth in SEQ ID NO.: 6; the heavy chain CDR amino acid sequences set forth in SEQ ID NO.: 5; or the heavy chain CDR amino acid sequences set forth in SEQ ID NO.: 6.

In one class of embodiments, the non-depleting CD4 antibody has a light chain amino acid sequence set forth in SEQ ID NO.: 1 and a heavy chain amino acid sequence set forth in SEQ ID NO.: 5; a light chain amino acid sequence set forth in SEQ ID NO.: 1 and a heavy chain amino acid sequence set forth in SEQ ID NO.: 6; a light chain amino acid sequence set forth in SEQ ID NO.: 2 and a heavy chain amino acid sequence set forth in SEQ ID NO.: 5; or a light chain amino acid sequence set forth in SEQ ID NO.: 2 and a heavy chain amino acid sequence set forth in SEQ ID NO.: 6.

In one class of embodiments, the non-depleting CD4 antibody comprises a light chain variable region amino acid sequence set forth in SEQ ID NO.: 1 and a heavy chain variable region amino acid sequence set forth in SEQ ID NO.: 5; a light chain variable region amino acid sequence set forth in SEQ ID NO.: 1 and a heavy chain variable region amino acid sequence set forth in SEQ ID NO.: 6; a light chain variable region amino acid sequence set forth in SEQ ID NO.: 2 and a heavy chain variable region amino acid sequence set forth in SEQ ID NO.: 5; or a light chain variable region amino acid sequence set forth in SEQ ID NO.: 2 and a heavy chain variable region amino acid sequence set forth in SEQ ID NO.: 6.

In one class of embodiments, the non-depleting CD4 antibody comprises the light chain CDR amino acid sequences set forth in SEQ ID NO.: 1 and the heavy chain CDR amino acid sequences set forth in SEQ ID NO.: 5; the light chain CDR amino acid sequences set forth in SEQ ID NO.: 1 and the heavy chain CDR amino acid sequences set forth in SEQ ID NO.: 6; the light chain CDR amino acid sequences set forth in SEQ ID NO.: 2 and the heavy chain CDR amino acid sequences set forth in SEQ ID NO.: 5; or the light chain CDR amino acid sequences set forth in SEQ ID NO.: 2 and the heavy chain CDR amino acid sequences set forth in SEQ ID NO.: 6.

In one class of embodiments, the non-depleting CD4 antibody comprises CDRL1 (SEQ ID NO.: 7), CDRL2 (SEQ ID NO.: 8) and CDRL3 (SEQ ID NO.: 9). In one class of embodiments, the non-depleting CD4 antibody comprises CDRH1 (SEQ ID NO.: 10), CDRH2 (SEQ ID NO.: 11), and CDRH3 (SEQ ID NO.: 12). In one class of embodiments, the non-depleting CD4 antibody comprises CDRL1 (SEQ ID NO.: 7), CDRL2 (SEQ ID NO.: 8), CDRL3 (SEQ ID NO.: 9), CDRH1 (SEQ ID NO.: 10), CDRH2 (SEQ ID NO.: 11), and CDRH3 (SEQ ID NO.: 12).

In one aspect, the invention provides methods of treating an autoimmune disease in a mammalian subject, e.g., a human subject, by administering a non-depleting CD4 antibody as described above in combination with at least a second compound. In certain embodiments, a second compound is a disease-modifying anti-rheumatic drug (DMARD), a corticosteroid, or a nonsteroidal antiinflammatory drug (NSAID). Suitable DMARDs include, but are not limited to, methotrexate, leflunomide, sulfasalazine, and hydroxychloroquine.

In another aspect, methods of treating an autoimmune disease in a mammalian subject, e.g., a human subject, as described above and who previously failed at least one biologic agent are provided. In certain embodiments, the biologic agent is adalimumab, etanercept, infliximab, golimumab, certolizumab pegol, rituximab, or ocrelizumab.

In another aspect, methods of treating an autoimmune disease in a mammalian subject, e.g., a human subject, as described above and who previously failed at least one DMARD are provided. In certain embodiments, the DMARD is methotrexate, leflunomide, sulfasalazine, or hydroxychloroquine.

The invention also provides methods of treating an autoimmune disease in a mammalian subject, e.g., a human subject, by administering subcutaneously a therapeutically effective amount of a non-depleting CD4 antibody that has been modified to increase serum half-life compared to the antibody without the modification at a dose between 0.2 mg/kg and 10 mg/kg in combination with an interstitial drug dispersion agent. In another aspect, the invention provides methods of treating an autoimmune disease in a mammalian subject, e.g., a human subject, by administering subcutaneously a therapeutically effective amount of a non-depleting CD4 antibody that has been modified to increase serum half-life compared to the antibody without the modification at a flat dose between 150 mg and 350 mg in combination with an interstitial drug dispersion agent. In certain embodiments, the interstitial drug dispersion agent is a soluble neutral-active hyaluronidase glycoprotein, including, but not limited to, rHuPH20.

In addition, the invention provides methods of treating an autoimmune disease in a mammalian subject, e.g., a human subject, by administering subcutaneously to the subject a first administration of a therapeutically effective amount of a non-depleting CD4 antibody that has been modified to increase serum half-life compared to the antibody without the modification at a dose between 0.05 mg/kg and 35 mg/kg, and by administering subcutaneously to the subject at least one subsequent administration of the modified non-depleting CD4 antibody at the same dose as the first administration with each subsequent administration being administered between five and nine days after the previous administration, the first administration and each subsequent administration administered in combination with an interstitial drug dispersion agent. In addition, the invention provides methods of treating an autoimmune disease in a mammalian subject, e.g., a human subject, by administering subcutaneously to the subject a first administration of a therapeutically effective amount of a non-depleting CD4 antibody that has been modified to increase serum half-life compared to the antibody without the modification at a flat dose between 150 mg and 350 mg, and by administering subcutaneously to the subject at least one subsequent administration of the modified non-depleting CD4 antibody at the same dose as the first administration with each subsequent administration being administered between five and nine days after the previous administration, the first administration and each subsequent administration administered in combination with an interstitial drug dispersion agent. In certain embodiments, the interstitial drug dispersion agent is a soluble neutral-active hyaluronidase glycoprotein, including, but not limited to, rHuPH20.

In yet another aspect, the invention provides a formulation comprising a therapeutically effective amount of a non-depleting CD4 antibody that has been modified to increase serum half-life compared to the antibody without the modification and an interstitial drug dispersion agent. In certain embodiments, the therapeutically effective amount of the non-depleting CD4 antibody in the formulation is between 150 mg and 350 mg, or between 200 mg and 300 mg, or between 225 mg and 275 mg. In certain embodiments, the therapeutically effective amount of the non-depleting CD4 antibody in the formulation is 250 mg. In certain embodiments, the interstitial drug dispersion agent in the formulation is a soluble neutral-active hyaluronidase glycoprotein, including, but not limited to, rHuPH20. In certain embodiments, the formulation is a pharmaceutical formulation.

In another aspect, the invention provides methods of treating an autoimmune disease in a mammalian subject, e.g., a human subject, by administering subcutaneously with a self-inject device a therapeutically effective amount of a non-depleting CD4 antibody that has been modified to increase serum half-life compared to the antibody without the modification at a dose between 0.2 mg/kg and 10 mg/kg. In another aspect, the invention provides methods of treating an autoimmune disease in a mammalian subject, e.g., a human subject, by administering subcutaneously with a self-inject device a therapeutically effective amount of a non-depleting CD4 antibody that has been modified to increase serum half-life compared to the antibody without the modification at a flat dose between 150 mg and 350 mg. In certain embodiments, a self-inject device includes, but is not limited to, a prefilled syringe, microneedle device, and needle-free injection device.

In yet another aspect, the invention provides methods of treating an autoimmune disease in a mammalian subject, e.g., a human subject, by administering subcutaneously with a self-inject device a first administration of a therapeutically effective amount of a non-depleting CD4 antibody that has been modified to increase serum half-life compared to the antibody without the modification at a dose between 0.05 mg/kg and 35 mg/kg, and by administering subcutaneously with a self-inject device at least one subsequent administration of the modified non-depleting CD4 antibody at the same dose as the first administration with each subsequent administration being administered between five and nine days after the previous administration. In yet another aspect, the invention provides methods of treating an autoimmune disease in a mammalian subject, e.g., a human subject, by administering subcutaneously with a self-inject device a first administration of a therapeutically effective amount of a non-depleting CD4 antibody that has been modified to increase serum half-life compared to the antibody without the modification at a flat dose between 150 mg and 350 mg, and by administering subcutaneously with a self-inject device at least one subsequent administration of the modified non-depleting CD4 antibody at the same dose as the first administration with each subsequent administration being administered between five and nine days after the previous administration. In certain embodiments, a self-inject device includes, but is not limited to, a prefilled syringe, microneedle device, and needle-free injection device.

The invention also provides a non-depleting CD4 antibody, wherein the antibody contains a modification to increase serum half-life compared to the antibody without the modification, for use in treating an autoimmune disease in a mammalian subject, the treatment comprising administering to the subject a therapeutically effective amount of the antibody subcutaneously at a dose between 0.2 mg/kg and 10 mg/kg. In addition, the invention provides for use of a non-depleting CD4 antibody, wherein the antibody contains a modification to increase serum half-life compared to the antibody without the modification in the preparation of a medicament for the treatment of an autoimmune disease in a mammalian subject, wherein the medicament is for administration subcutaneously at a dose between 0.2 mg/kg and 10 mg/kg. The invention still further provides a formulation for subcutaneous administration comprising a dose of between 0.2 mg/kg and 10 mg/kg of a non-depleting CD4 antibody, wherein the antibody contains a modification to increase serum half-life compared to the antibody without the modification. In certain embodiments, the dose is selected from 1.5 mg/kg, 2.0 mg/kg, 3.0 mg/kg, 3.5 mg/kg, and 5.0 mg/kg.

The invention also provides a non-depleting CD4 antibody, wherein the antibody contains a modification to increase serum half-life compared to the antibody without the modification, for use in treating an autoimmune disease in a mammalian subject, the treatment comprising administering to the subject a therapeutically effective amount of the antibody subcutaneously at a flat dose between 150 mg and 350 mg. The invention further provides for use of a non-depleting CD4 antibody, wherein the antibody contains a modification to increase serum half-life compared to the antibody without the modification in the preparation of a medicament for the treatment of an autoimmune disease in a mammalian subject, wherein the medicament is for administration subcutaneously at a flat dose between 150 mg and 350 mg. In addition, the invention provides a formulation for subcutaneous administration comprising a flat dose between 150 mg and 350 mg of a non-depleting CD4 antibody, wherein the antibody contains a modification to increase serum half-life compared to the antibody without the modification. In certain embodiments, the flat dose is between 200 mg and 300 mg. In certain embodiments, the flat dose is between 225 mg and 275 mg. In certain embodiments, the flat dose is 250 mg.

In another aspect, the invention provides a non-depleting CD4 antibody, wherein the antibody contains a modification to increase serum half-life compared to the antibody without the modification, for use in treating an autoimmune disease in a mammalian subject, wherein the treatment comprises administering to the subject a therapeutically effective amount of the antibody, wherein administration of the antibody comprises a first administration and at least one subsequent administration, wherein the first administration is at a dose between 0.05 mg/kg and 35 mg/kg and each subsequent administration is at the same dose as the first administration, wherein each subsequent administration is administered between five and nine days after the previous administration, and wherein the first administration and each subsequent administration are administered subcutaneously. The invention also provides for use of a non-depleting CD4 antibody, wherein the antibody contains a modification to increase serum half-life compared to the antibody without the modification, in the preparation of a medicament for the treatment of an autoimmune disease in a mammalian subject, wherein the medicament is for administration comprising a first administration and at least one subsequent administration, wherein the first administration is at a dose between 0.05 mg/kg and 35 mg/kg and each subsequent administration is at the same dose as the first administration, wherein each subsequent administration is for administration between five and nine days after the previous administration, and wherein the first administration and each subsequent administration are for administration subcutaneously. The invention further provides a formulation for subcutaneous administration comprising a dose of between 0.05 mg/kg and 35 mg/kg of a non-depleting CD4 antibody, wherein the antibody contains a modification to increase serum half-life compared to the antibody without the modification. In certain embodiments, the dose is selected from 1.5 mg/kg, 2.0 mg/kg, 3.0 mg/kg, 3.5 mg/kg, and 5.0 mg/kg.

In another aspect, the invention provides a non-depleting CD4 antibody, wherein the antibody contains a modification to increase serum half-life compared to the antibody without the modification, for use in treating an autoimmune disease in a mammalian subject, wherein the treatment comprises administering to the subject a therapeutically effective amount of the antibody, wherein administration of the antibody comprises a first administration and at least one subsequent administration, wherein the first administration is a flat dose between 150 mg and 350 mg and each subsequent administration is at the same dose as the first administration, wherein each subsequent administration is administered between five and nine days after the previous administration, and wherein the first administration and each subsequent administration are administered subcutaneously. The invention also provides for use of a non-depleting CD4 antibody, wherein the antibody contains a modification to increase serum half-life compared to the antibody without the modification, in the preparation of a medicament for the treatment of an autoimmune disease in a mammalian subject, wherein the medicament is for administration comprising a first administration and at least one subsequent administration, wherein the first administration is a flat dose between 150 mg and 350 mg and each subsequent administration is at the same dose as the first administration, wherein each subsequent administration is for administration between five and nine days after the previous administration, and wherein the first administration and each subsequent administration are for administration subcutaneously. In certain embodiments, the flat dose is between 200 mg and 300 mg, or between 225 mg and 275 mg. In certain embodiments, the flat dose is 250 mg.

The invention also provides a non-depleting CD4 antibody for use as described above, and wherein the antibody is administered in combination with at least a second compound selected from a DMARD, a corticosteroid, and a NSAID. In addition, the invention provides for use of a non-depleting CD4 antibody in the preparation of a medicament as described above, wherein the medicament is for administration in combination with at least a second compound selected from a DMARD, a corticosteroid, and a NSAID. In another aspect, the invention provides for use of a non-depleting CD4 antibody, wherein the antibody contains a modification to increase serum half-life compared to the antibody without the modification, in combination with at least a second compound selected from a DMARD, a corticosteroid, and a NSAID for the preparation of a medicament for the treatment of an autoimmune disease in a mammalian subject, wherein the medicament is for administration subcutaneously at a dose between 0.2 mg/kg and 10 mg/kg. The invention further provides for use of a non-depleting CD4 antibody, wherein the antibody contains a modification to increase serum half-life compared to the antibody without the modification, in combination with at least a second compound selected from a DMARD, a corticosteroid, and a NSAID for the preparation of a medicament for the treatment of an autoimmune disease in a mammalian subject, wherein the medicament is for administration subcutaneously at a flat dose between 150 mg and 350 mg.

In yet another aspect, the invention provides for use of a non-depleting CD4 antibody, wherein the antibody contains a modification to increase serum half-life compared to the antibody without the modification, in combination with at least a second compound selected from a DMARD, a corticosteroid, and a NSAID for the preparation of a medicament for the treatment of an autoimmune disease in a mammalian subject, wherein the medicament is for administration comprising a first administration and at least one subsequent administration, wherein the first administration is at a dose between 0.05 mg/kg and 35 mg/kg and each subsequent administration is at the same dose as the first administration, wherein each subsequent administration is for administration between five and nine days after the previous administration, and wherein the first administration and each subsequent administration are for administration subcutaneously. In certain embodiments, the dose is selected from 1.5 mg/kg, 2.0 mg/kg, 3.0 mg/kg, 3.5 mg/kg, and 5.0 mg/kg. In addition, the invention provides for use of a non-depleting CD4 antibody, wherein the antibody contains a modification to increase serum half-life compared to the antibody without the modification, in combination with at least a second compound selected from a DMARD, a corticosteroid, and a NSAID for the preparation of a medicament for the treatment of an autoimmune disease in a mammalian subject, wherein the medicament is for administration comprising a first administration and at least one subsequent administration, wherein the first administration is at a flat dose between 150 mg and 350 mg and each subsequent administration is at the same dose as the first administration, wherein each subsequent administration is for administration between five and nine days after the previous administration, and wherein the first administration and each subsequent administration are for administration subcutaneously. In certain embodiments, the flat dose is between 200 mg and 300 mg, or between 225 mg and 275 mg. In certain embodiments, the flat dose is 250 mg.

In another aspect, the invention provides a non-depleting CD4 antibody, wherein the antibody contains a modification to increase serum half-life compared to the antibody without the modification, for use in treating an autoimmune disease in a mammalian subject, wherein the treatment comprises administering to the subject a therapeutically effective amount of the antibody subcutaneously at a dose between 0.2 mg/kg and 10 mg/kg in combination with an interstitial drug dispersion agent. Also provided is a non-depleting CD4 antibody, wherein the antibody contains a modification to increase serum half-life compared to the antibody without the modification, for use in treating an autoimmune disease in a mammalian subject, wherein the treatment comprises administering to the subject a therapeutically effective amount of the antibody subcutaneously at a dose between 0.2 mg/kg and 10 mg/kg, wherein the treatment further comprises administration in combination with an interstitial drug dispersion agent. In yet another aspect, the invention provides a non-depleting CD4 antibody, wherein the antibody contains a modification to increase serum half-life compared to the antibody without the modification, in combination with an interstitial drug dispersion agent for use in treating an autoimmune disease in a mammalian subject, wherein the treatment comprises administering to the subject a therapeutically effective amount of the antibody subcutaneously at a dose between 0.2 mg/kg and 10 mg/kg. In certain embodiments, the dose is selected from 1.5 mg/kg, 2.0 mg/kg, 3.0 mg/kg, 3.5 mg/kg, and 5.0 mg/kg.

In another aspect, the invention provides a non-depleting CD4 antibody, wherein the antibody contains a modification to increase serum half-life compared to the antibody without the modification, for use in treating an autoimmune disease in a mammalian subject, wherein the treatment comprises administering to the subject a therapeutically effective amount of the antibody subcutaneously at a flat dose between 150 mg and 350 mg in combination with an interstitial drug dispersion agent. Also provided is a non-depleting CD4 antibody, wherein the antibody contains a modification to increase serum half-life compared to the antibody without the modification, for use in treating an autoimmune disease in a mammalian subject, wherein the treatment comprises administering to the subject a therapeutically effective amount of the antibody subcutaneously at a flat dose between 150 mg and 350 mg, wherein the treatment further comprises administration in combination with an interstitial drug dispersion agent. In yet another aspect, the invention provides a non-depleting CD4 antibody, wherein the antibody contains a modification to increase serum half-life compared to the antibody without the modification, in combination with an interstitial drug dispersion agent for use in treating an autoimmune disease in a mammalian subject, wherein the treatment comprises administering to the subject a therapeutically effective amount of the antibody subcutaneously at a flat dose between 150 mg and 350 mg. In certain embodiments, the flat dose is between 200 mg and 300 mg, or between 225 mg and 275 mg. In certain embodiments, the flat dose is 250 mg.

Also provided is use of a non-depleting CD4 antibody, wherein the antibody contains a modification to increase serum half-life compared to the antibody without the modification, in the preparation of a medicament for the treatment of an autoimmune disease in a mammalian subject, wherein the medicament is for administration subcutaneously at a dose between 0.2 mg/kg and 10 mg/kg in combination with an interstitial drug dispersion agent. In another aspect, the invention provides for use of a non-depleting CD4 antibody, wherein the antibody contains a modification to increase serum half-life compared to the antibody without the modification, in combination with an interstitial drug dispersion agent for the preparation of a medicament for the treatment of an autoimmune disease in a mammalian subject, wherein the medicament is for administration subcutaneously at a dose between 0.2 mg/kg and 10 mg/kg. In certain embodiments, the dose is selected from 1.5 mg/kg, 2.0 mg/kg, 3.0 mg/kg, 3.5 mg/kg, and 5.0 mg/kg.

Also provided is use of a non-depleting CD4 antibody, wherein the antibody contains a modification to increase serum half-life compared to the antibody without the modification, in the preparation of a medicament for the treatment of an autoimmune disease in a mammalian subject, wherein the medicament is for administration subcutaneously at a flat dose between 150 mg and 350 mg in combination with an interstitial drug dispersion agent. In another aspect, the invention provides for use of a non-depleting CD4 antibody, wherein the antibody contains a modification to increase serum half-life compared to the antibody without the modification, in combination with an interstitial drug dispersion agent for the preparation of a medicament for the treatment of an autoimmune disease in a mammalian subject, wherein the medicament is for administration subcutaneously at a flat dose between 150 mg and 350 mg. In certain embodiments, the flat dose is between 200 mg and 300 mg, or between 225 mg and 275 mg. In certain embodiments, the flat dose is 250 mg.

In another aspect, the invention provides a non-depleting CD4 antibody, wherein the antibody contains a modification to increase serum half-life compared to the antibody without the modification, for use in treating an autoimmune disease in a mammalian subject, wherein the treatment comprises administering to the subject a therapeutically effective amount of the antibody, wherein administration of the antibody comprises a first administration and at least one subsequent administration, wherein the first administration is at a dose between 0.05 mg/kg and 35 mg/kg and each subsequent administration is at the same dose as the first administration, wherein each subsequent administration is administered between five and nine days after the previous administration, and wherein the first administration and each subsequent administration are administered subcutaneously in combination with an interstitial drug dispersion agent. The invention also provides for use of a non-depleting CD4 antibody, wherein the antibody contains a modification to increase serum half-life compared to the antibody without the modification, in the preparation of a medicament for the treatment of an autoimmune disease in a mammalian subject, wherein the medicament is for administration comprising a first administration and at least one subsequent administration, wherein the first administration is at a dose between 0.05 mg/kg and 35 mg/kg and each subsequent administration is at the same dose as the first administration, wherein each subsequent administration is for administration between five and nine days after the previous administration, and wherein the first administration and each subsequent administration are for administration subcutaneously in combination with an interstitial drug dispersion agent. In certain embodiments, the dose is selected from 1.5 mg/kg, 2.0 mg/kg, 3.0 mg/kg, 3.5 mg/kg, and 5.0 mg/kg.

In another aspect, the invention provides a non-depleting CD4 antibody, wherein the antibody contains a modification to increase serum half-life compared to the antibody without the modification, for use in treating an autoimmune disease in a mammalian subject, wherein the treatment comprises administering to the subject a therapeutically effective amount of the antibody, wherein administration of the antibody comprises a first administration and at least one subsequent administration, wherein the first administration is at a flat dose between 150 mg and 350 mg and each subsequent administration is at the same dose as the first administration, wherein each subsequent administration is administered between five and nine days after the previous administration, and wherein the first administration and each subsequent administration are administered subcutaneously in combination with an interstitial drug dispersion agent. The invention also provides for use of a non-depleting CD4 antibody, wherein the antibody contains a modification to increase serum half-life compared to the antibody without the modification, in the preparation of a medicament for the treatment of an autoimmune disease in a mammalian subject, wherein the medicament is for administration comprising a first administration and at least one subsequent administration, wherein the first administration is at a flat dose between 150 mg and 350 mg and each subsequent administration is at the same dose as the first administration, wherein each subsequent administration is for administration between five and nine days after the previous administration, and wherein the first administration and each subsequent administration are for administration subcutaneously in combination with an interstitial drug dispersion agent. In certain embodiments, the flat dose is between 200 mg and 300 mg, or between 225 mg and 275 mg. In certain embodiments, the flat dose is 250 mg.

In another aspect, the invention provides a non-depleting CD4 antibody, wherein the antibody contains a modification to increase serum half-life compared to the antibody without the modification, for use in treating an autoimmune disease in a mammalian subject, wherein the treatment comprises administering to the subject a therapeutically effective amount of the antibody subcutaneously at a dose between 0.2 mg/kg and 10 mg/kg in combination with a self-inject device. Also provided is a non-depleting CD4 antibody, wherein the antibody, wherein the antibody contains a modification to increase serum half-life compared to the antibody without the modification, for use in a method of treating an autoimmune disease in a mammalian subject, wherein the treatment comprises administering to the subject a therapeutically effective amount of the antibody subcutaneously at a dose between 0.2 mg/kg and 10 mg/kg, wherein the treatment further comprises administration in combination with a self-inject device. In yet another aspect, the invention provides a non-depleting CD4 antibody, wherein the antibody contains a modification to increase serum half-life compared to the antibody without the modification, in combination with a self-inject device for use in treating an autoimmune disease in a mammalian subject, wherein the treatment comprises administering to the subject a therapeutically effective amount of the antibody subcutaneously at a dose between 0.2 mg/kg and 10 mg/kg. In certain embodiments, the dose is selected from 1.5 mg/kg, 2.0 mg/kg, 3.0 mg/kg, 3.5 mg/kg, and 5.0 mg/kg.

In another aspect, the invention provides a non-depleting CD4 antibody, wherein the antibody contains a modification to increase serum half-life compared to the antibody without the modification, for use in treating an autoimmune disease in a mammalian subject, wherein the treatment comprises administering to the subject a therapeutically effective amount of the antibody subcutaneously at a flat dose between 150 mg and 350 mg in combination with a self-inject device. Also provided is a non-depleting CD4 antibody, wherein the antibody, wherein the antibody contains a modification to increase serum half-life compared to the antibody without the modification, for use in a method of treating an autoimmune disease in a mammalian subject, wherein the treatment comprises administering to the subject a therapeutically effective amount of the antibody subcutaneously at a flat dose between 150 mg and 350 mg, wherein the treatment further comprises administration in combination with a self-inject device. In yet another aspect, the invention provides a non-depleting CD4 antibody, wherein the antibody contains a modification to increase serum half-life compared to the antibody without the modification, in combination with a self-inject device for use in treating an autoimmune disease in a mammalian subject, wherein the treatment comprises administering to the subject a therapeutically effective amount of the antibody subcutaneously at a flat dose between 150 mg and 350 mg. In certain embodiments, the flat dose is between 200 mg and 300 mg, or between 225 mg and 275 mg. In certain embodiments, the flat dose is 250 mg.

Also provided is use of a non-depleting CD4 antibody, wherein the antibody contains a modification to increase serum half-life compared to the antibody without the modification, in the preparation of a medicament for the treatment of an autoimmune disease in a mammalian subject, wherein the medicament is for administration subcutaneously at a dose between 0.2 mg/kg and 10 mg/kg in combination with a self-inject device. In another aspect, the invention provides for use of a non-depleting CD4 antibody, wherein the antibody contains a modification to increase serum half-life compared to the antibody without the modification, in combination with a self-inject device for the preparation of a medicament for the treatment of an autoimmune disease in a mammalian subject, wherein the medicament is for administration subcutaneously at a dose between 0.2 mg/kg and 10 mg/kg. In certain embodiments, the dose is selected from 1.5 mg/kg, 2.0 mg/kg, 3.0 mg/kg, 3.5 mg/kg, and 5.0 mg/kg.

Also provided is use of a non-depleting CD4 antibody, wherein the antibody contains a modification to increase serum half-life compared to the antibody without the modification, in the preparation of a medicament for the treatment of an autoimmune disease in a mammalian subject, wherein the medicament is for administration subcutaneously at a flat dose between 150 mg and 350 mg in combination with a self-inject device. In another aspect, the invention provides for use of a non-depleting CD4 antibody, wherein the antibody contains a modification to increase serum half-life compared to the antibody without the modification, in combination with a self-inject device for the preparation of a medicament for the treatment of an autoimmune disease in a mammalian subject, wherein the medicament is for administration subcutaneously at a flat dose between 150 mg and 350 mg. In certain embodiments, the flat dose is between 200 mg and 300 mg, or between 225 mg and 275 mg. In certain embodiments, the flat dose is 250 mg.

In another aspect, the invention provides a non-depleting CD4 antibody, wherein the antibody contains a modification to increase serum half-life compared to the antibody without the modification, for use in treating an autoimmune disease in a mammalian subject, wherein the treatment comprises administering to the subject a therapeutically effective amount of the antibody, wherein administration of the antibody comprises a first administration and at least one subsequent administration, wherein the first administration is at a dose between 0.05 mg/kg and 35 mg/kg and each subsequent administration is at the same dose as the first administration, wherein each subsequent administration is administered between five and nine days after the previous administration, and wherein the first administration and each subsequent administration are administered subcutaneously in combination with a self-inject device. The invention also provides for use of a non-depleting CD4 antibody, wherein the antibody contains a modification to increase serum half-life compared to the antibody without the modification, in the preparation of a medicament for the treatment of an autoimmune disease in a mammalian subject, wherein the medicament is for administration comprising a first administration and at least one subsequent administration, wherein the first administration is at a dose between 0.05 mg/kg and 35 mg/kg and each subsequent administration is at the same dose as the first administration, wherein each subsequent administration is for administration between five and nine days after the previous administration, and wherein the first administration and each subsequent administration are for administration subcutaneously in combination with a self-inject device. In certain embodiments, the dose is selected from 1.5 mg/kg, 2.0 mg/kg, 3.0 mg/kg, 3.5 mg/kg, and 5.0 mg/kg.

In another aspect, the invention provides a non-depleting CD4 antibody, wherein the antibody contains a modification to increase serum half-life compared to the antibody without the modification, for use in treating an autoimmune disease in a mammalian subject, wherein the treatment comprises administering to the subject a therapeutically effective amount of the antibody, wherein administration of the antibody comprises a first administration and at least one subsequent administration, wherein the first administration is at a flat dose between 150 mg and 350 mg and each subsequent administration is at the same dose as the first administration, wherein each subsequent administration is administered between five and nine days after the previous administration, and wherein the first administration and each subsequent administration are administered subcutaneously in combination with a self-inject device. The invention also provides for use of a non-depleting CD4 antibody, wherein the antibody contains a modification to increase serum half-life compared to the antibody without the modification, in the preparation of a medicament for the treatment of an autoimmune disease in a mammalian subject, wherein the medicament is for administration comprising a first administration and at least one subsequent administration, wherein the first administration is at a flat dose between 150 mg and 350 mg and each subsequent administration is at the same dose as the first administration, wherein each subsequent administration is for administration between five and nine days after the previous administration, and wherein the first administration and each subsequent administration are for administration subcutaneously in combination with a self-inject device. In certain embodiments, the flat dose is between 200 mg and 300 mg, or between 225 mg and 275 mg. In certain embodiments, the flat dose is 250 mg.

### BRIEF DESCRIPTION OF THE DRAWINGS

FIG. 1 shows non-depleting anti-CD4 light chain variants discussed in the Examples. Amino acid sequences of CDR sequences are underlined. The first amino acid of the constant region is shown in bold and underlined. (A) non-depleting anti-CD4 light chain variant number 1 (SEQ ID NO.: 1); (B) non-depleting anti-CD4 light chain variant number 2 (SEQ ID NO.: 2).

FIG. 2 shows non-depleting anti-CD4 heavy chain variants discussed in the Examples. Amino acid sequences of CDR sequences are underlined. The first amino acid of the first constant region is shown in bold and underlined. (A) non-depleting anti-CD4 heavy chain variant number 1 (SEQ ID NO.: 3); (B) non-depleting anti-CD4 heavy chain variant number 2 (SEQ ID NO.: 4); (C) non-depleting anti-CD4 heavy chain variant number 3 (SEQ ID NO.: 5); (D) non-depleting anti-CD4 heavy chain variant number 4 (SEQ ID NO.: 6).

FIG. 3 shows binding plots for non-depleting anti-CD4 antibody variant D and control monoclonal antibodies to Fcγ receptors (FcγR) as described in Example 1. (A) binding plot for antibodies to FcγR IA; (B) binding plot for antibodies to FcγR IIA; (C) binding plot for antibodies to FcγR IIB; (D) binding plot for antibodies to FcγR IIIA-F158; (E) binding plot for antibodies to FcγR IIIA-V158.

FIG. 4 shows ADCC assay results with peripheral blood mononuclear cells and two human T-lymphoma cell lines, Jurkat and Hut-78, analyzed by flow cytometry as described in Example 1. (A) Cell surface expression of CD4 analyzed by flow cytometry; (B) ADCC curves from one representative experiment in which anti-CD4 variants were assayed with Hut-78 cells.

FIG. 5 shows in vivo clearance of non-depleting anti-CD4 antibody variants in baboons following intravenous administration as described in Example 1.

FIG. 6 shows mean and individual estimated time CD4 T-cell receptor sites reach 10% CD4-free in baboons given Variant B, Variant C, or Variant D as described in Example 1.

FIG. 7 shows serum concentration of non-depleting anti-CD4 antibody Variant D over time in baboons following repeated intravenous or subcutaneous administration of antibody as described in Example 2.

FIG. 8 shows light chain and heavy chain CDR sequences of non-depleting anti-CD4 light chain and heavy chain variants discussed in the Examples. (A) Non-depleting anti-CD4 CDRL1 (SEQ ID NO.: 7); (B) Non-depleting anti-CD4 CDRL2 (SEQ ID NO.: 8); (C) Non-depleting anti-CD4 CDRL3 (SEQ ID NO.: 9); (D) Non-depleting anti-CD4 CDRH1 (SEQ ID NO.: 10); (E) Non-depleting anti-CD4 CDRH2 (SEQ ID NO.: 11); (F) Non-depleting anti-CD4 CDRH3 (SEQ ID NO.: 12).

FIG. 9 shows amino acid sequences of (A) histidine-tagged human FcRn (SEQ ID NO.: 13) and (B) histidine-tagged baboon FcRn (SEQ ID NO.: 14) used in FcRn binding affinity studies as described in Example 1.

FIG. 10 shows serum concentration of non-depleting anti-CD4 antibody Variant D over time in RA patients in the single ascending-dose study as described in Example 3. LLOQ (lower limit of quantification) of the assay is indicated in the figure.

FIG. 11 shows the percentage of CD4 receptor occupancy (A) and cell surface CD4 receptor expression (B) relative to baseline in the peripheral blood of RA patients in the single ascending-dose study as described in Example 3.

FIG. 12 shows the predicted PK and PD time-profiles of Variant D in RA patients following weekly SC injections of Variant D at 3.5 mg/kg as described in Example 3.

FIG. 13 shows the results of sorting Th1 and Th17 cells from a fresh leukapheresis mononuclear preparation (A) and cytokine secretion by the sorted Th1 and Th17 cells (B) as described in Example 4.

FIG. 14 shows inhibition of human Th1 and Th17 CD4+ cells by Variant D or Control Ig in a mixed lymphocyte reaction as described in Example 4.

### DETAILED DESCRIPTION OF CERTAIN EMBODIMENTS

The invention provides isolated antibodies that bind to CD4 and methods of using the same, e.g., for the diagnosis or treatment of autoimmune disorders including, but not limited to, lupus, multiple sclerosis, and rheumatoid arthritis.

### I. Certain Definitions

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Any methods and materials similar or equivalent to those described herein can be used in the practice for testing of the present invention, and non-limiting materials and methods are described herein. Accordingly, the terminology used herein is for the purpose of describing particular embodiments only, and is not intended to be limiting.

As used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like.

Ranges provided in the specification and appended claims include both end points and all points between the end points. Thus, for example, a range of 2.0 to 3.0 includes 2.0, 3.0, and all points between 2.0 and 3.0.

The term “autoimmune disease” refers to a disease or disorder arising from and/or directed against an individual's own tissues or organs, or a co-segregate or manifestation thereof, or resulting condition therefrom. Typically, various clinical and laboratory markers of autoimmune diseases may exist including, but not limited to, hypergammaglobulinemia, high levels of autoantibodies, antigen-antibody complex deposits in tissues, clinical benefit from corticosteroid or immunosuppressive treatments, and lymphoid cell aggregates in affected tissues.

“Lupus” refers to an autoimmune disease or disorder involving antibodies that attack connective tissue. The principal form of lupus is a systemic one, systemic lupus erythematosus (SLE), which in certain instances include cutaneous involvement. “Lupus” as used herein includes SLE as well as other types of lupus (including, e.g., cutaneous lupus erythematosus (CLE), lupus nephritis (LN), extrarenal, cerebritis, pediatric, non-renal, discoid, and alopecia).

“Multiple sclerosis” (MS) is an autoimmune demyelinating disorder. MS generally exhibits a relapsing-remitting course or a chronic progressive course.

As used herein, “relapsing-remitting MS” (RRMS) is characterized by partial or total recovery after attacks.

The term “secondary-progressive MS” (SPMS) refers to a relapsing-remitting course of MS which becomes steadily progressive. Attacks and partial recoveries may continue to occur.

The term “primary-progressive MS” (PPMS) refers to MS that is progressive from the onset. Symptoms in patients with PPMS generally do not remit—i.e., decrease in intensity.

“Rheumatoid arthritis” (RA) refers to a chronic systemic autoimmune inflammatory disease that mainly involves the synovial membrane of multiple joints with resultant injury to the articular cartilage, resulting in joint destruction. The main presenting symptoms in RA are pain, stiffness, swelling, and/or loss of function of one or more joints.

A “subject” herein is typically a human. In certain embodiments, a subject is a non-human mammal. Exemplary non-human mammals include laboratory, domestic, pet, sport, and stock animals, e.g., mice, cats, dogs, horses, and cows. Typically, the subject is eligible for treatment, e.g., treatment of an autoimmune disorder, treatment related to a tissue transplant, or the like.

As used herein, “lifetime” of a subject refers to the remainder of the life of the subject after starting treatment.

“Treatment” of a subject refers to therapeutic treatment. Treatment also refers to prophylactic or preventative measures. Those in need of treatment include those already with an autoimmune disease, such as lupus, MS, rheumatoid arthritis, or inflammatory bowel disease, as well as those in which the autoimmune disease is to be prevented. Thus, the subject may have been diagnosed as having an autoimmune disease, such as lupus, MS, rheumatoid arthritis, or inflammatory bowel disease, or may be predisposed or susceptible to the autoimmune disease.

The term “ameliorates” or “amelioration” as used herein refers to a decrease, reduction or elimination of a condition, disease, disorder, or phenotype, including an abnormality or symptom.

A “symptom” of a disease or disorder (e.g., an autoimmune disease, such as, for example, lupus, MS, rheumatoid arthritis) is any morbid phenomenon or departure from the normal in structure, function, or sensation, experienced by a subject and indicative of disease.

The expression “therapeutically effective amount” refers to an amount that is effective for preventing, ameliorating, or treating a disease or disorder (e.g., lupus, MS, rheumatoid arthritis, or inflammatory bowel disease). For example, a “therapeutically effective amount” of an antibody refers to an amount of the antibody that is effective for preventing, ameliorating, or treating the specified disease or disorder. Similarly, a “therapeutically effective amount” of a combination of an antibody and a second compound refers to an amount of the antibody and an amount of the second compound that, in combination, is effective for preventing, ameliorating, or treating the specified disease or disorder.

It is to be understood that the terminology “a combination of” two compounds does not mean that the compounds have to be administered in admixture with each other. Thus, treatment with or use of such a combination encompasses a mixture of the compounds or separate administration of the compounds, and includes administration on the same day or different days. Thus the terminology “combination” means two or more compounds are used for the treatment, either individually or in admixture with each other. When an antibody and a second compound, for example, are administered in combination to a subject, the antibody is present in the subject at a time when the second compound is also present in the subject, whether the antibody and second compound are administered individually or in admixture to the subject. In certain embodiments, a compound other than the antibody is administered prior to the antibody. In certain embodiments, a compound other than the antibody is administered after the antibody.

The CD4 antigen, or “CD4,” is a glycoprotein expressed on the surface of T lymphocytes, as well as certain other cells. Other names for CD4 in the art include cluster of differentiation 4 and L3T4. CD4 is described, for example, in entry 186940 in the Online Mendelian Inheritance in Man database, on the world wide web at www.ncbi.nlm.nih.gov/Omim.

A “CD4 antibody” or an “anti-CD4 antibody” or an “antibody that binds to CD4” refer to an antibody that is capable of binding CD4 with sufficient affinity such that the antibody is useful as a diagnostic and/or therapeutic agent in targeting CD4. In certain embodiments, the extent of binding of an anti-CD4 antibody to an unrelated, non-CD4 protein is less than about 10% of the binding of the antibody to CD4 as measured, e.g., by a radioimmunoassay (RIA). In certain embodiments, an antibody that binds to CD4 has a dissociation constant (Kd) of ≦1 μM, ≦100 nM, ≦10 nM, ≦1 nM, or ≦0.1 nM. In certain embodiments, an anti-CD4 antibody binds to an epitope of CD4 that is conserved among CD4 from different species. As used herein, a “CD4 antibody,” an “anti-CD4 antibody,” and an “anti-CD4” are equivalent terms and are used interchangeably.

A “non-depleting CD4 antibody,” used interchangeably with “non-depleting anti-CD4 antibody” is a CD4 antibody that depletes less than 50% of CD4+ cells. CD4+ cells are quantified by various methods known in the art, for example, by flow cytometry, e.g., as described in the Examples herein. In certain embodiments, a non-depleting CD4 antibody depletes less than 25% of CD4+ cells. In certain embodiments, a non-depleting CD4 antibody depletes less than 10% of CD4+ cells. In certain embodiments, i.e. in a clinical setting, treatment with a non-depleting CD4 antibody does not result in CD4+ T-cell counts below 250 cells/mm3. Conversely, a “depleting CD4 antibody,” used interchangeably with “depleting anti-CD4 antibody” is a CD4 antibody that depletes 50% or more of CD4+ cells, or even 75% or more or 90% or more of CD4+ cells. Depletion of CD4+ cells (e.g., reduction in circulating CD4+ cell levels in a subject treated with the antibody) can be achieved by various mechanisms, such as antibody-dependent cell-mediated cytotoxicity, complement-dependent cytotoxicity, inhibition of T-cell proliferation, and/or induction of T-cell death.

The term “antibody” herein is used in the broadest sense and specifically covers monoclonal antibodies, polyclonal antibodies, multispecific antibodies (e.g. bispecific antibodies) formed from at least two intact antibodies, chimeric antibodies, human antibodies, and antibody fragments so long as they exhibit the desired biological activity (e.g., CD4 binding). An antibody is a protein comprising one or more polypeptides substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes.

An “isolated” antibody is one which has been identified and separated and/or recovered from a component of its natural environment. Contaminant components of its natural environment are materials which would interfere with research, diagnostic or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. Isolated antibody includes the antibody in situ within recombinant cells since at least one component of the antibody's natural environment will not be present. Ordinarily, however, isolated antibody will be prepared by at least one purification step.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

“Antibody fragments” comprise a portion of an intact antibody. Antibody fragments, in certain instances, comprises the antigen-binding region of the intact antibody. Examples of antibody fragments include Fab, Fab′, F(ab′)₂, and Fv fragments; diabodies; linear antibodies; single-chain antibody molecules; and multispecific antibodies formed from antibody fragments.

An “intact antibody” is one comprising heavy- and light-variable domains as well as an Fc region.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light-chain and the heavy-chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al. Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody-dependent cell-mediated cytotoxicity.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment that contains a complete antigen-recognition and antigen-binding site. This region consists of a dimer of one heavy-chain and one light-chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy-chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments that have hinge cysteines between them. Other chemical couplings of antibody fragments are also known. See, e.g., Fundamental Immunology, W. E. Paul, ed., Raven Press, N.Y. (1999), for a more detailed description of other antibody fragments.

While various antibody fragments are defined in terms of the digestion of an intact antibody, one of skill will appreciate that such fragments may be synthesized de novo either chemically or by utilizing recombinant DNA methodology. Thus, the term antibody, as used herein, includes antibodies or fragments thereof either produced by the modification of whole antibodies or synthesized de novo using recombinant DNA methodologies.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include PRIMATIZED® antibodies comprising variable-domain antigen-binding sequences derived from a non-human primate (e.g. Old World Monkey, such as baboon, rhesus, or cynomolgus monkey) and human constant-region sequences (U.S. Pat. No. 5,693,780).

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a HVR of the recipient are replaced by residues from a HVR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1: 105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE technology). See also, for example, Li et al., Proc. Natl. Acad. Sci. USA, 103:3557-3562 (2006) regarding human antibodies generated via a human B-cell hybridoma technology.

The term “hypervariable region,” “HVR,” or “HV,” when used herein refers to the regions of an antibody variable domain which are hypervariable in sequence and/or form structurally defined loops. Generally, antibodies comprise six HVRs; three in the VH(H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

Loop Kabat AbM Chothia Contact

L1 L24-L34 L24-L34 L26-L32 L30-L36

L2 L50-L56 L50-L56 L50-L52 L46-L55

L3 L89-L97 L89-L97 L91-L96 L89-L96

H1 H31-H35B H26-H35B H26-H32H30-H35B
(Kabat Numbering)

H1 H31-H35H26-H35H26-H32H30-H35
(Chothia Numbering)

H2H50-H65H50-H58H53-H55H47-H58

H3H95-H102 H95-H102 H96-H101 H93-H101

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

“Framework” or “FR” residues are those variable domain residues other than the HVR residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies means residue numbering by the Kabat numbering system. Unless stated otherwise herein, references to residue numbers in the constant domain of antibodies means residue numbering by the EU numbering system (e.g., see International Patent Application No. PCT/US05/047072 [International Publication No. WO 2006/073941], Figures for EU numbering).

An “affinity matured” antibody is one with one or more alterations in one or more HVRs thereof which result in an improvement in the affinity of the antibody for antigen, compared to a parent antibody which does not possess those alteration(s). In one embodiment, an affinity matured antibody has nanomolar or even picomolar affinities for the target antigen. Affinity matured antibodies may be produced using certain procedures known in the art. For example, Marks et al. Bio/Technology 10:779-783 (1992) describes affinity maturation by VH and VL domain shuffling. Random mutagenesis of HVR and/or framework residues is described by, for example, Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

A “functional Fc region” possesses an “effector function” of a native sequence Fc region. Exemplary “effector functions” include C1q binding; CDC; Fc receptor binding; ADCC; phagocytosis; down regulation of cell surface receptors (e.g. B cell receptor; BCR), etc. Such effector functions generally require the Fc region to be combined with a binding domain (e.g., an antibody variable domain) and can be assessed using various assays as disclosed, for example, in definitions herein.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, e.g., one or more amino acid substitution(s). In certain embodiments, the variant Fc region has at least one amino acid substitution compared to a native sequence Fc region or to the Fc region of a parent polypeptide, e.g. from about one to about ten amino acid substitutions, or from about one to about five amino acid substitutions in a native sequence Fc region or in the Fc region of the parent polypeptide. In certain embodiments, the variant Fc region herein will possess at least about 80% homology with a native sequence Fc region and/or with an Fc region of a parent polypeptide, at least about 90% homology therewith, or at least about 95% homology therewith.

“Fc receptor” or “FcR” describes a receptor that binds to the Fc region of an antibody. In some embodiments, an FcR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see, e.g., Daëron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which, in certain instances, is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

Binding to human FcRn in vivo and serum half life of human FcRn high affinity binding polypeptides can be assayed, e.g., in transgenic mice or transfected human cell lines expressing human FcRn, or in primates to which the polypeptides with a variant Fc region are administered. WO 2000/42072 (Presta) describes antibody variants with improved or diminished binding to FcRs. See also, e.g., Shields et al. J. Biol. Chem. 9(2):6591-6604 (2001).

The term “serum clearance” refers to a pharmacokinetic measurement of the disappearance of an antibody from the serum of a subject following administration of the antibody. Various methods for determining clearance are known in the art, including those described in the Examples herein.

A “CD4 binding fragment” of an antibody is a fragment of the antibody that retains the ability to bind CD4. As noted, the fragment is optionally produced by digestion of the intact antibody or synthesized de novo.

An “epitope” is the specific region of an antigenic molecule that binds to an antibody.

The phrase “substantially similar,” or “substantially the same”, as used herein, denotes a sufficiently high degree of similarity between two numeric values (generally one associated with an antibody of the invention and the other associated with a reference/comparator antibody) such that one of skill in the art would consider the difference between the two values to be of little or no biological and/or statistical significance within the context of the biological characteristic measured by said values (e.g., Kd values). The difference between said two values is, for example, less than about 50%, less than about 40%, less than about 30%, less than about 20%, and/or less than about 10% as a function of the reference/comparator value.

The phrase “substantially reduced,” or “substantially different,” as used herein, denotes a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., Kd values). The difference between said two values is, for example, greater than about 10%, greater than about 20%, greater than about 30%, greater than about 40%, and/or greater than about 50% as a function of the value for the reference/comparator molecule.

“Binding affinity” of an antibody for an antigen generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative embodiments are described in the following.

In one embodiment, the “Kd” or “Kd value” according to this invention is measured by a radiolabeled antigen binding assay (RIA) performed with the Fab version of an antibody of interest and its antigen as described by the following assay that measures solution binding affinity of Fabs for antigen by equilibrating Fab with a minimal concentration of -labeled antigen in the presence of a titration series of unlabeled antigen, then capturing bound antigen with an anti-Fab antibody-coated plate (Chen et al. (1999) J. Mol. Biol 293:865-881). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbant plate (Nunc #269620), 100 pM or 26 pM [125I]-antigen are mixed with serial dilutions of a Fab of interest (e.g., consistent with assessment of an anti-VEGF antibody, Fab-12, in Presta et al. (1997) Cancer Res. 57:4593-4599). The Fab of interest is then incubated overnight; however, the incubation may continue for a longer period (e.g., 65 hours) to insure that equilibrium is reached. Thereafter, the mixtures are transferred to the capture plate for incubation at room temperature (e.g., for one hour). The solution is then removed and the plate washed eight times with 0.1% Tween-20 in PBS. When the plates have dried, 150 μl/well of scintillant (MicroScint™-20; Packard) is added, and the plates are counted on a Topcount® gamma counter (Packard) for ten minutes. Concentrations of each Fab that give less than or equal to 20% of maximal binding are chosen for use in competitive binding assays. According to another embodiment, the Kd or Kd value is measured by using surface plasmon resonance assays using a BIAcore®-2000 or a BIAcore®-3000 (BIAcore, Inc., Piscataway, N.J.) at 25° C. with immobilized antigen CM5 chips at ˜10 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, into 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% Tween 20 (PBST) at 25° C. at a flow rate of approximately 25 μl/min. Association rates (kon) and dissociation rates (koff) are calculated using a simple one-to-one Langmuir binding model (BIAcore® Evaluation Software version 3.2) by simultaneously fitting the association and dissociation sensorgram. The equilibrium dissociation constant (Kd) is calculated as the ratio koff/kon. See, e.g., Chen et al. (1999) J. Mol. Biol 293:865-881. If the on-rate exceeds 10⁶ M⁻¹ S⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-Aminco® spectrophotometer (ThermoSpectronic) with a stirred cuvette.

An “on-rate,” “rate of association,” “association rate,” or “kₒₙ” according to this invention can also be determined as described above using a BIACORE®-2000 or a BIACORE®-3000 system (BIAcore, Inc., Piscataway, N.J.).

“Binding” e.g., of an antibody for a receptor, e.g., FcR, reflects a relative binding affinity and may be expressed as an IC₅₀ value. Various methods are known in the art for determining IC₅₀ including those described in the Examples herein.

An antibody variant with “altered” FcR binding affinity or ADCC activity is one which has either enhanced or diminished FcR binding activity and/or ADCC activity compared to a parent or unmodified antibody or to an antibody comprising a native sequence Fc region. The antibody variant which “displays increased binding” to an FcR binds at least one FcR with better affinity than the parent or unmodified antibody or to an antibody comprising a native sequence Fc region. The antibody variant which “displays decreased binding” to an FcR, binds at least one FcR with worse affinity than the parent or unmodified antibody or to an antibody comprising a native sequence Fc region. Such variants which display decreased binding to an FcR may possess little or no appreciable binding to an FcR, e.g., 0-20% binding to the FcR compared to a native sequence IgG Fc region, e.g. as determined in the Examples herein.

An antibody variant which binds an FcR, e.g., FcRn, with “better affinity” or increased “binding affinity” compared to a parent or unmodified antibody or to an antibody comprising a native sequence Fc region, is one which binds any one or more of the above identified FcRs, e.g., FcRn, with substantially better binding affinity than the parent or unmodified antibody, when the amounts of antibody variant and parent or unmodified antibody in the binding assay are essentially the same. For example, the antibody variant with improved or increased FcR binding affinity may display from between 1.15 fold and 100 fold, or between 1.2 fold and 50 fold, or between 1.5 fold and 10 fold, or between 2.0 fold and 4.5 fold improvement in FcR binding affinity compared to the parent or unmodified antibody, where FcR binding affinity, e.g., FcRn binding affinity, is determined, for example, as disclosed in the Examples herein. In certain embodiments, binding affinity is a relative affinity determined by quantifying binding of a variant antibody to a receptor, e.g., FcRn, relative to the binding of a parent or unmodified antibody to the receptor. In certain such embodiments, binding is an IC₅₀ value, as disclosed in the Examples herein.

An “amino acid sequence” is a polymer of amino acid residues (a protein, polypeptide, etc.) or a character string representing an amino acid polymer, depending on context.

The term “immunosuppressive agent” as used herein for therapy refers to substances that act to suppress or mask the immune system of the mammal being treated herein. This would include substances that suppress cytokine production, down-regulate or suppress self-antigen expression, or mask the MHC antigens. Examples of such agents include 2-amino-6-aryl-5-substituted pyrimidines (see U.S. Pat. No. 4,665,077); nonsteroidal antiinflammatory drugs (NSAIDs); ganciclovir, tacrolimus, glucocorticoids such as cortisol or aldosterone, anti-inflammatory agents such as a cyclooxygenase inhibitor, a 5-lipoxygenase inhibitor, or a leukotriene receptor antagonist; purine antagonists such as azathioprine or mycophenolate mofetil (MMF); alkylating agents such as cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as corticosteroids or glucocorticosteroids or glucocorticoid analogs, e.g., prednisone, methylprednisolone, and dexamethasone; dihydrofolate reductase inhibitors such as methotrexate (oral or subcutaneous); hydroxycloroquine; sulfasalazine; leflunomide; cytokine or cytokine receptor antibodies including anti-interferon-alpha, -beta, or -gamma antibodies, anti-tumor necrosis factor-alpha antibodies (golimumab, certolizumab pegol, infliximab or adalimumab), anti-TNF-alpha immunoadhesin (etanercept), anti-tumor necrosis factor-beta antibodies, anti-interleukin-2 antibodies and anti-IL-2 receptor antibodies; anti-LFA-1 antibodies, including anti-CD11a and anti-CD18 antibodies; heterologous anti-lymphocyte globulin; pan-T antibodies, including anti-CD3; soluble peptide containing a LFA-3 binding domain (WO 1990/08187 published Jul. 26, 1990); streptokinase; TGF-beta; streptodornase; RNA or DNA from the host; FK506; RS-61443; deoxyspergualin; rapamycin; T-cell receptor (Cohen et al., U.S. Pat. No. 5,114,721); T-cell-receptor fragments (Offner et al., Science, 251: 430-432 (1991); WO 1990/11294; Ianeway, Nature, 341: 482 (1989); and WO 1991/01133); and T-cell-receptor antibodies (EP 340,109) such as T10B9.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small-molecule toxins or enzymatically active toxins of bacterial, fungal, plant, or animal origin, or fragments thereof.

The term “cytokine” is a generic term for proteins released by one cell population that act on another cell as intercellular mediators. Examples of such cytokines are lymphokines, monokines; interleukins (ILs) such as IL-1, IL-1a, IL-2, IL-3, IL-4, IL-5, IL-6, IL-7, IL-8, IL-9, IL-11, IL-12, IL-115; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native-sequence cytokines, including synthetically produced small-molecule entities and pharmaceutically acceptable derivatives and salts thereof.

The term “hormone” refers to polypeptide hormones, which are generally secreted by glandular organs with ducts. Included among the hormones are, for example, growth hormone such as human growth hormone, N-methionyl human growth hormone, and bovine growth hormone; parathyroid hormone; thyroxine; insulin; proinsulin; relaxin; prorelaxin; glycoprotein hormones such as follicle-stimulating hormone (FSH), thyroid-stimulating hormone (TSH), and luteinizing hormone (LH); prolactin, placental lactogen, mouse gonadotropin-associated peptide, inhibin; activin; mullerian-inhibiting substance; and thrombopoietin. As used herein, the term hormone includes proteins from natural sources or from recombinant cell culture and biologically active equivalents of the native-sequence hormone, including synthetically produced small-molecule entities and pharmaceutically acceptable derivatives and salts thereof.

The term “growth factor” refers to proteins that promote growth, and include, for example, hepatic growth factor; fibroblast growth factor; vascular endothelial growth factor; nerve growth factors such as NGF-β; platelet-derived growth factor; transforming growth factors (TGFs) such as TGF-α and TGF-β; insulin-like growth factor-I and -II; erythropoietin (EPO); osteoinductive factors; interferons such as interferon-α, -β, and -γ; and colony-stimulating factors (CSFs) such as macrophage-CSF (M-CSF); granulocyte-macrophage-CSF (GM-CSF); and granulocyte-CSF (G-CSF). As used herein, the term growth factor includes proteins from natural sources or from recombinant cell culture and biologically active equivalents of the native-sequence growth factor, including synthetically produced small-molecule entities and pharmaceutically acceptable derivatives and salts thereof.

For the purposes herein, “tumor necrosis factor-alpha (TNF-alpha)” refers to a human TNF-alpha molecule comprising the amino acid sequence as described in Pennica et al., Nature, 312:721 (1984) or Aggarwal et al., JBC, 260:2345 (1985).

A “TNF-alpha inhibitor” herein is an agent that inhibits, to some extent, a biological function of TNF-alpha, generally through binding to TNF-alpha and neutralizing its activity. Examples of TNF inhibitors specifically contemplated herein are etanercept (ENBREL®), infliximab (REMICADE®), adalimumab (HUMIRA®), golimumab (SIMPONI™), and certolizumab pegol (CIMZIA®).

Examples of “nonsteroidal anti-inflammatory drugs” or “NSAIDs” are acetylsalicylic acid, ibuprofen, naproxen, indomethacin, sulindac, tolmetin, including salts and derivatives thereof, etc.

The term “integrin” refers to a receptor protein that allows cells both to bind to and to respond to the extracellular matrix and is involved in a variety of cellular functions such as wound healing, cell differentiation, homing of tumor cells, and apoptosis. They are part of a large family of cell adhesion receptors that are involved in cell-extracellular matrix and cell-cell interactions. Functional integrins consist of two transmembrane glycoprotein subunits, called alpha and beta, which are non-covalently bound. The alpha subunits all share some homology to each other, as do the beta subunits. The receptors always contain one alpha chain and one beta chain. Examples include α6β1, α3β1, α7β1, LFA-1 etc. As used herein, the term integrin includes proteins from natural sources or from recombinant cell culture and biologically active equivalents of the native-sequence integrin, including synthetically produced small-molecule entities and pharmaceutically acceptable derivatives and salts thereof. An “α4-integrin” is the α4 subunit of α4-β1 and α4-β7 integrins that are expressed on the surface of leukocytes other than neutrophils.

Examples of “integrin antagonists or antibodies” herein include an LFA-1 antibody, such as efalizumab (RAPTIVA®) commercially available from Genentech, or an alpha 4 integrin antibody (e.g., a “α4-integrin antibody” is an antibody that binds α4-integrin) such as natalizumab (TYSABRI®) available from Biogen, or diazacyclic phenylalanine derivatives (WO 2003/89410), phenylalanine derivatives (WO 2003/70709, WO 2002/28830, WO 2002/16329 and WO 2003/53926), phenylpropionic acid derivatives (WO 2003/10135), enamine derivatives (WO 2001/79173), propanoic acid derivatives (WO 2000/37444), alkanoic acid derivatives (WO 2000/32575), substituted phenyl derivatives (U.S. Pat. Nos. 6,677,339 and 6,348,463), aromatic amine derivatives (U.S. Pat. No. 6,369,229), ADAM disintegrin domain polypeptides (US 2002/0042368), antibodies to alphavbeta3 integrin (EP 633945), aza-bridged bicyclic amino acid derivatives (WO 2002/02556), etc.

“Corticosteroid” refers to any one of several synthetic or naturally occurring substances with the general chemical structure of steroids that mimic or augment the effects of the naturally occurring corticosteroids. Examples of synthetic corticosteroids include prednisone, prednisolone (including methylprednisolone), dexamethasone triamcinolone, and betamethasone.

A “B-cell surface marker” or “B-cell surface antigen” herein is an antigen expressed on the surface of a B cell that can be targeted with an antagonist that binds thereto. Exemplary B-cell surface markers include the CD10, CD 19, CD20, CD21, CD22, CD23, CD24, CD37, CD40, CD53, CD72, CD73, CD74, CDw75, CDw76, CD77, CDw78, CD79a, CD79b, CD80, CD81, CD82, CD83, CDw84, CD85, and CD86 leukocyte surface markers (for descriptions, see The Leukocyte Antigen Facts Book, 2nd Edition. 1997, ed. Barclay et al. Academic Press, Harcourt Brace & Co., New York). Other B-cell surface markers include RP105, FcRH2, B-cell CR2, CCR6, P2X5, HLA-DOB, CXCR5, FCER2, BR3, Btig, NAG14, SLGC16270, FcRH1, IRTA2, ATWD578, FcRH3, IRTA1, FcRH6, BCMA, and 239287. The B-cell surface marker of particular interest is preferentially expressed on B cells compared to other non-B-cell tissues of a mammal and may be expressed on both precursor B cells and mature B cells.

An “antibody that binds to a B-cell surface marker” is a molecule that, upon binding to a B-cell surface marker, destroys or depletes B cells in a mammal and/or interferes with one or more B-cell functions, e.g. by reducing or preventing a humoral response elicited by the B cell. The antibody in certain instances is able to deplete B cells (i.e. reduce circulating B-cell levels) in a mammal treated therewith. Such depletion may be achieved via various mechanisms such as antibody-dependent cell-mediated cytotoxicity (ADCC) and/or complement-dependent cytotoxicity (CDC), inhibition of B-cell proliferation, and/or induction of B-cell death (e.g. via apoptosis).

An “antagonist” refers to a molecule capable of neutralizing, blocking, inhibiting, abrogating, reducing or interfering with the activities of a particular or specified protein, including its binding to one or more receptors in the case of a ligand or binding to one or more ligands in case of a receptor. Antagonists include antibodies and antigen-binding fragments thereof, proteins, peptides, glycoproteins, glycopeptides, glycolipids, polysaccharides, oligosaccharides, nucleic acids, bioorganic molecules, peptidomimetics, pharmacological agents and their metabolites, transcriptional and translation control sequences, and the like. Antagonists also include small molecule inhibitors of the protein, and fusion proteins, receptor molecules and derivatives which bind specifically to the protein thereby sequestering its binding to its target, antagonist variants of the protein, antisense molecules directed to the protein, RNA aptamers, and ribozymes against the protein.

A “B-cell surface marker antagonist” is a molecule that, upon binding to a B-cell surface marker, destroys or depletes B cells in a mammal and/or interferes with one or more B-cell functions, e.g. by reducing or preventing a humoral response elicited by the B cell. The antagonist in certain instances is able to deplete B cells (i.e. reduce circulating B-cell levels) in a mammal treated therewith. Such depletion may be achieved via various mechanisms such as ADCC and/or CDC, inhibition of B-cell proliferation, and/or induction of B-cell death (e.g. via apoptosis). Exemplary antagonists include synthetic or native-sequence peptides, fusion proteins, and small-molecule antagonists that bind to the B-cell marker, optionally conjugated with or fused to a cytotoxic agent. Examples include but are not limited to, e.g., CD20 antibodies, BR3 antibodies (e.g., WO0224909), BR3-Fc, etc.

Examples of CD20 antibodies include: “C2B8,” which is now called “rituximab” (“RITUXAN®”) (U.S. Pat. No. 5,736,137); the yttrium-[90]-labeled 2B8 murine antibody designated “Y2B8” or “Ibritumomab Tiuxetan” (ZEVALIN®) commercially available from IDEC Pharmaceuticals, Inc. (U.S. Pat. No. 5,736,137; 2B8 deposited with ATCC under accession no. HB11388 on Jun. 22, 1993); murine IgG2a “B1,” also called “Tositumomab,” optionally labeled with ¹³¹I, to generate the “¹³¹I-B1” or “iodine I¹³¹ tositumomab” antibody (BEXXAR™) commercially available from Corixa (see, also, U.S. Pat. No. 5,595,721); murine monoclonal antibody “1F5” (Press et al. Blood 69(2):584-591 (1987) and variants thereof including “framework-patched” or humanized 1F5 (WO 2003/002607, Leung, S.; ATCC deposit HB-96450); murine 2H7 and chimeric 2H7 antibody (U.S. Pat. No. 5,677,180); humanized 2H7 (see, e.g., WO04/056312; US20060024295); HUMAX-CD20™ antibodies (Genmab, Denmark); the human monoclonal antibodies set forth in WO 2004/035607 (Teeling et al.); AME-133™ antibodies (Applied Molecular Evolution); A20 antibody or variants thereof such as chimeric or humanized A20 antibody (cA20, hA20, respectively) (US 2003/0219433, Immunomedics); and monoclonal antibodies L27, G28-2, 93-1 B3, B-C1 or NU-B2 available from the International Leukocyte Typing Workshop (Valentine et al., In: Leukocyte Typing III (McMichael, Ed., p. 440, Oxford University Press (1987)).

Examples of “disease-modifying anti-rheumatic drugs” or “DMARDs” include hydroxycloroquine, sulfasalazine, methotrexate (plus oral and subcutaneous methrotrexate), leflunomide, azathioprine, D-penicillamine, Gold (oral), Gold (intramuscular), minocycline, cyclosporine, Staphylococcal protein A immunoadsorption, including salts and derivatives thereof, etc.

“CTLA4” is expressed on activated T lymphocytes and is involved in down-regulation of the immune response. Other names for CTLA4 in the literature include cytotoxic T-lymphocyte-associated antigen 4, cytotoxic T-lymphocyte-associated protein 4, cell differentiation antigen CD152, and cytotoxic T-lymphocyte-associated granule serine protease 4.

An “interstitial drug dispersion agent” refers to an agent, such as an enzyme, capable of degrading the interstitial matrix.

The term “soluble neutral-active hyaluronidase glycoprotein” or “sHASEGP” refers to a hyaluronidase, an enzyme capable of degrading glycosaminoglycan. One such hyaluronidase is PH20, the predominant hyaluronidase in mammalian testes. PH20 is a neutral pH-active hyaluronidase and degrades glycosaminoglycans under physiologic conditions. “rHuPH20” is a recombinant and soluble form of human hyaluronidase lacking the glycosyl-phosphatidylinositol moiety.

A “self-inject device” refers to a medical device for self-administration, e.g., by a patient or in-home caregiver, of a therapeutic agent. Self-inject devices include autoinjector devices and other devices designed for self-administration.

A variety of additional terms are defined or otherwise characterized herein.

### II. Compositions and Methods

Antibodies that bind to CD4 are provided. Antibodies of the invention are useful, e.g., for the diagnosis or treatment of autoimmune disorders. In certain embodiments, antibodies of the invention are useful for the diagnosis or treatment of lupus, multiple sclerosis, or rheumatoid arthritis. In certain embodiments, antibodies of the invention are non-depleting.

CD4 is a surface glycoprotein primarily expressed on cells of the T lymphocyte lineage, including a majority of thymocytes and a subset of peripheral T cells. Low levels of CD4 are also expressed by some non-lymphoid cells, although the functional significance of such divergent cellular distribution is unknown. On mature T cells, CD4 serves a co-recognition function through interaction with MHC Class II molecules expressed in antigen presenting cells. CD4+ T cells constitute primarily the helper subset which regulates T and B cell functions during T cell-dependent responses to viral, bacterial, fungal and parasitic infections.

During the pathogenesis of autoimmune diseases, in particular when tolerance to self antigens breaks down, CD4+ T cells can contribute to inflammatory responses which result in joint and tissue destruction. These processes are facilitated, e.g., by the recruitment of inflammatory cells of the hematopoietic lineage, production of antibodies, inflammatory cytokines and mediators, and by the activation of killer cells.

CD4+ T cells have been implicated in the pathogenesis of lupus. For example, CD4+ T cells are present in sites of glomerulonephritis. CD4+ T cells from SLE patients are reported to be hyper-responsive to antigen and resistant to apoptosis in vitro. Autoantigen-specific CD4+ T cells that can support production of autoantibodies by B cells (effector/memory CD4+ cells that produce IFN-γ) are present in SLE patients. In addition, a strong association between MHC Class II alleles and risk for SLE is observed.

CD4+ T cells have been similarly implicated in the pathogenesis of MS. For example, CD4+ helper T cells are involved in the pathogenesis of MS and a corresponding laboratory model, experimental allergic encephalomyelitis (EAE), and laboratory animals depleted of T cells exhibit a loss of ability to develop EAE (U.S. Pat. No. 4,695,459 to Steinman et al. entitled “Method of treating autoimmune diseases that are mediated by Leu3/CD4 phenotype T cells”, Traugott et al. (1983) “Multiple sclerosis: distribution of T cell subsets within active chronic lesions” Science 219:308-310, Amason et al. (1962) “Role of the thymus in immune reaction in rats: II. Suppressive effect of thymectomy at birth on reactions of delayed (cellular) hypersensitivity and the circulating small lymphocyte” J Exp Med 116:177-186, and Gonatas and Howard (1974) “Inhibition of experimental allergic encephalomyelitis in rats severely depleted of T cells” Science 186:839-841). CD4+ and CD8+ T cells are found in MS lesions; both are known to produce inflammatory cytokines, although their relative contribution to pathogenesis has not been determined. A four-fold increase is observed in the frequency of myelin-specific CD4+ cells in blood of MS patients. Several drugs currently used or which mostly will be used for treatment of MS are believed to work, in part, through their action on T cells; for example, Tysabri® (natalizumab, alpha-4 integrin antibody), CAMPATH® (alemtuzumab, CD52 antibody), and daclizumab (IL-2Rαantibody). In addition, an increased risk of MS is associated with MHC Class II alleles (3.6 fold) and, to a lesser extent, Class I alleles (2 fold).

In addition, CD4+ T cells have been implicated in the pathogenesis of RA. RA is characterized by a cell-mediated immune response in both synovial and extra-synovial sites in patients with the disease. The synovial tissue of patients with RA is infiltrated by large numbers of lymphocytes and monocytes. Of the T-cell infiltrates, those expressing CD4 appear to be the predominate subtype (Janossy et al., Lancet 2 (8251):839-42, 1981; Pitzalis et al., Clin. Immunol. Immunopathol. 45:252-52, 1987; Pitzalis et al., Eur. J. Immunol. 21:369-76, 1991). These T cells demonstrate evidence of activation through expression of activation markers, such as the interleukin-2 (IL-2) receptor, MHC class II molecules, and CD69. Through T-cell activation and related interaction with other inflammatory cells, there is also the increased production of inflammatory cytokines. Animal models of RA have demonstrated the importance of T cells in disease manifestation as well as attenuation of the disease with anti-T cell therapies (Chu and Londei, J. Immunol. 157:2685-89, 1996). In patients with RA, previous therapies (both depleting and non-depleting monoclonal antibodies) directed against T cells expressing CD4 have shown evidence of immunomodulatory effects and clinical improvement (Choy et al., Rheumatol. 41:1142-1148, 2002; Mason et al., J. Rheumatol. 29:220-29, 2002; Luggen et al., abstract presented at the 2003 Annual European Congress of Rheumatology, Ann. Rheum. Dis. OPO110).

In one aspect, the present invention provides methods of treating an autoimmune disease by administering a non-depleting CD4 antibody, alone or in combination with another compound used clinically or experimentally to treat the autoimmune disease. As used herein, an autoimmune disease refers to a disease or disorder arising from and/or directed against an individual's own tissues or organs, or a co-segregate or manifestation thereof, or resulting condition therefrom. Typically, various clinical and laboratory markers of autoimmune diseases may exist including, but not limited to, hypergammaglobulinemia, high levels of autoantibodies, antigen-antibody complex deposits in tissues, clinical benefit from corticosteroid or immunosuppressive treatments, and lymphoid cell aggregates in affected tissues.

An autoimmune disease can be an organ-specific disease (i.e., the immune response is specifically directed against an organ system such as the endocrine system, the hematopoietic system, the skin, the cardiopulmonary system, the gastrointestinal and liver systems, the renal system, the thyroid, the ears, the neuromuscular system, the central nervous system, etc.) or a systemic disease which can affect multiple organ systems (for example, systemic lupus erythematosus (SLE), rheumatoid arthritis, polymyositis, etc.). Exemplary diseases include autoimmune rheumatologic disorders (such as, for example, rheumatoid arthritis, Sjögren's syndrome, scleroderma, lupus such as SLE and lupus nephritis, polymyositis/dermatomyositis, cryoglobulinemia, anti-phospholipid antibody syndrome, and psoriatic arthritis), autoimmune gastrointestinal and liver disorders (such as, for example, inflammatory bowel diseases (e.g., ulcerative colitis and Crohn's disease), autoimmune gastritis and pernicious anemia, autoimmune hepatitis, primary biliary cirrhosis, primary sclerosing cholangitis, and celiac disease), vasculitis (such as, for example, ANCA-negative vasculitis and ANCA-associated vasculitis, including Churg-Strauss vasculitis, Wegener's granulomatosis, and microscopic polyangiitis), autoimmune neurological disorders (such as, for example, multiple sclerosis, opsoclonus myoclonus syndrome, myasthenia gravis, neuromyelitis optica, Parkinson's disease, Alzheimer's disease, and autoimmune polyneuropathies), renal disorders (such as, for example, glomerulonephritis, Goodpasture's syndrome, and Berger's disease), autoimmune dermatologic disorders (such as, for example, psoriasis, urticaria, hives, pemphigus vulgaris, bullous pemphigoid, and cutaneous lupus erythematosus), hematologic disorders (such as, for example, thrombocytopenic purpura, thrombotic thrombocytopenic purpura, post-transfusion purpura, and autoimmune hemolytic anemia), atherosclerosis, uveitis, autoimmune hearing diseases (such as, for example, inner ear disease and hearing loss), Behcet's disease, Raynaud's syndrome, organ transplant, and autoimmune endocrine disorders (such as, for example, diabetic-related autoimmune diseases such as insulin-dependent diabetes mellitus (IDDM), Addison's disease, and autoimmune thyroid disease (e.g., Graves' disease and thyroiditis)).

Specific examples of other autoimmune disorders as defined herein, which in some cases encompass those listed above, include, but are not limited to, arthritis (acute and chronic, rheumatoid arthritis including juvenile-onset rheumatoid arthritis and stages such as rheumatoid synovitis, gout or gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, osteoarthritis, arthritis chronica progrediente, arthritis deformans, polyarthritis chronica primaria, reactive arthritis, menopausal arthritis, estrogen-depletion arthritis, and ankylosing spondylitis/rheumatoid spondylitis), autoimmune lymphoproliferative disease, inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, hives, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, gastrointestinal inflammation, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, graft-versus-host disease, angioedema such as hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN(RPGN), proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, food allergies, drug allergies, insect allergies, rare allergic disorders such as mastocytosis, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, SLE, such as cutaneous SLE or subacute cutaneous SLE, neonatal lupus syndrome (NLE), and lupus erythematosus disseminatus, juvenile onset (Type I) diabetes mellitus, including pediatric IDDM, adult onset diabetes mellitus (Type II diabetes), autoimmune diabetes, idiopathic diabetes insipidus, diabetic retinopathy, diabetic nephropathy, diabetic colitis, diabetic large-artery disorder, immune responses associated with acute and delayed hypersensitivity mediated by cytokines and T-lymphocytes, tuberculosis, sarcoidosis, granulomatosis including lymphomatoid granulomatosis, agranulocytosis, vasculitides (including large-vessel vasculitis such as polymyalgia rheumatica and giant-cell (Takayasu's) arteritis, medium-vessel vasculitis such as Kawasaki's disease and polyarteritis nodosa/periarteritis nodosa, immunovasculitis, CNS vasculitis, cutaneous vasculitis, hypersensitivity vasculitis, necrotizing vasculitis such as fibrinoid necrotizing vasculitis and systemic necrotizing vasculitis, ANCA-negative vasculitis, and ANCA-associated vasculitis such as Churg-Strauss syndrome (CSS), Wegener's granulomatosis, and microscopic polyangiitis), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia(s), cytopenias such as pancytopenia, leukopenia, diseases involving leukocyte diapedesis, CNS inflammatory disorders, Alzheimer's disease, Parkinson's disease, multiple organ injury syndrome such as those secondary to septicemia, trauma or hemorrhage, antigen-antibody complex-mediated diseases, anti-glomerular basement membrane disease, anti-phospholipid antibody syndrome, motoneuritis, allergic neuritis, Behçet's disease/syndrome, Castleman's syndrome, Goodpasture's syndrome, Reynaud's syndrome, Sjögren's syndrome, Stevens-Johnson syndrome, pemphigoid or pemphigus such as pemphigoid bullous, cicatricial (mucous membrane) pemphigoid, skin pemphigoid, pemphigus vulgaris, paraneoplastic pemphigus, pemphigus foliaceus, pemphigus mucus-membrane pemphigoid, and pemphigus erythematosus, epidermolysis bullosa acquisita, ocular inflammation, including allergic ocular inflammation such as allergic conjunctivis, linear IgA bullous disease, autoimmune-induced conjunctival inflammation, autoimmune polyendocrinopathies, Reiter's disease or syndrome, thermal injury due to an autoimmune condition, preeclampsia, an immune complex disorder such as immune complex nephritis, antibody-mediated nephritis, neuroinflammatory disorders, polyneuropathies, chronic neuropathy such as IgM polyneuropathies or IgM-mediated neuropathy, thrombocytopenia (as developed by myocardial infarction patients, for example), including thrombotic thrombocytopenic purpura (TTP), post-transfusion purpura (PTP), heparin-induced thrombocytopenia, and autoimmune or immune-mediated thrombocytopenia including, for example, idiopathic thrombocytopenic purpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, Grave's eye disease (opthalmopathy or thyroid-associated opthalmopathy), polyglandular syndromes such as autoimmune polyglandular syndromes, for example, type I (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant-cell hepatitis, chronic active hepatitis or autoimmune chronic active hepatitis, pneumonitis such as lymphoid interstitial pneumonitis (LIP), bronchiolitis obliterans (non-transplant) vs. NSIP, Guillain-Barré syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia such as mixed cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, keratitis such as Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia greata, alopecia totalis, CREST syndrome (calcinosis, Raynaud's phenomenon, esophageal dysmotility, sclerodactyl), and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, fibrosing mediastinitis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis (systemic inflammatory response syndrome (SIRS)), endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant-cell polymyalgia, chronic hypersensitivity pneumonitis, conjunctivitis, such as vernal catarrh, keratoconjunctivitis sicca, and epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders (cerebral vascular insufficiency) such as arteriosclerotic encephalopathy and arteriosclerotic retinopathy, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica (sympathetic ophthalmitis), neonatal ophthalmitis, optic neuritis, orchitis granulomatosa, pancreatitis, polyradiculitis acuta, pyoderma gangrenosum, Quervain's thyreoiditis, acquired spenic atrophy, non-malignant thymoma, lymphofollicular thymitis, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, autoimmune polyendocrinopathies, oophoritis, primary myxedema, autoimmune atrophic gastritis, rheumatic diseases, mixed connective tissue disease, nephrotic syndrome, insulitis, polyendocrine failure, autoimmune polyglandular syndromes, including polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, allergic sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, spondyloarthropathies, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism such as chronic arthrorheumatism, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, and endometriosis.

In one aspect, the present invention also provides methods of treating lupus, including SLE and lupus nephritis, by administering a non-depleting CD4 antibody, alone or in combination with another compound used clinically or experimentally to treat lupus. Another aspect of the invention provides methods of treating lupus nephritis, including mid- to late-stage disease, by administration of a non-depleting CD4 antibody that results in an improvement in renal function and/or a reduction in proteinuria or active urinary sediment.

In one aspect, a subject is eligible for treatment for lupus, including treatment for SLE or lupus nephritis. For the purposes herein, such eligible subject is one that is experiencing or has experienced one or more signs, symptoms, or other indicators of lupus or has been diagnosed with lupus, whether, for example, newly diagnosed, previously diagnosed with a new flare, or chronically steroid dependent with a new flare, or is at risk for developing lupus. One eligible for treatment of lupus may optionally be identified as one who is screened by renal biopsy and/or is screened using an assay to detect auto-antibodies, such as those noted below, wherein autoantibody production is assessed qualitatively and/or quantitatively. In the case of patients eligible for treatment for SLE, SLE can be associated with the production of antinuclear antibodies, circulating immune complexes, and activation of the complement system. SLE has an incidence of about 1 in 700 women between the ages of 20 and 60. SLE can affect any organ system and can cause severe tissue damage. Numerous autoantibodies of differing specificity are present in SLE. SLE patients often produce autoantibodies having anti-DNA, anti-Ro, and anti-platelet specificity and that are capable of initiating clinical features of the disease, such as glomerulonephritis, arthritis, serositis, complete heart block in newborns, and hematologic abnormalities. These autoantibodies are also possibly related to central nervous system disturbances. Arbuckle et al. describes the development of autoantibodies before the clinical onset of SLE (Arbuckle et al. (2003) N. Engl. J. Med. 349(16):1526-1533). The presence of antibodies immunoreactive with double-stranded native DNA is frequently used as a diagnostic marker for SLE. Exemplary such auto-antibodies associated with SLE are anti-nuclear antibodies (Ab), anti-double-stranded DNA (dsDNA) Ab, anti-Sm Ab, anti-nuclear ribonucleoprotein Ab, anti-phospholipid Ab, anti-ribosomal P Ab, anti-Ro/SS-A Ab, anti-Ro Ab, and anti-La Ab.

Diagnosis of lupus (and determination of eligibility for treatment) can be performed as established in the art. For example, diagnosis of SLE may be according to current American College of Rheumatology (ACR) criteria. Active disease may be defined by one British Isles Lupus Activity Group's (BILAG) “A” criteria or two BILAG “B” criteria, e.g., as applied in U.S. patent application publication 2006/0024295 by Brunetta entitled “Method for treating lupus.” Some signs, symptoms, or other indicators used to diagnose SLE adapted from Tan et al. (1982) “The 1982 Revised Criteria for the Classification of SLE” Arth Rheum 25:1271-1277 may be malar rash such as rash over the cheeks, discoid rash, or red raised patches, photosensitivity such as reaction to sunlight, resulting in the development of or increase in skin rash, oral ulcers such as ulcers in the nose or mouth, usually painless, arthritis, such as non-erosive arthritis involving two or more peripheral joints (arthritis in which the bones around the joints do not become destroyed), serositis, pleuritis or pericarditis, renal disorder such as excessive protein in the urine (proteinuria, greater than 0.5 g (gram)/day or 3+on test sticks) and/or cellular casts (abnormal elements derived from the urine and/or white cells and/or kidney tubule cells), neurologic signs, symptoms, or other indicators, seizures (convulsions), and/or psychosis in the absence of drugs or metabolic disturbances that are known to cause such effects, and hematologic signs, symptoms, or other indicators such as hemolytic anemia or leukopenia (white bloodcount below 4,000 cells per cubic millimeter) or lymphopenia (less than 1,500 lymphocytes per cubic millimeter) or thrombocytopenia (less than 100,000 platelets per cubic millimeter). The leukopenia and lymphopenia must be detected on two or more occasions. The thrombocytopenia must be detected in the absence of drugs known to induce it. The invention is not limited to these signs, symptoms, or other indicators of lupus.

A nephritic lupus flare can be defined as 1) an increase of >30% in Scr within a 1-month period, or 2) a recurrence or appearance of nephrotic syndrome, or 3) a 3-fold increase in urinary protein with baseline proteinuria>1 g/24 hrs or as noted in U.S. patent application publication 2006/0024295. For lupus nephritis, the treatment eligibility may be evidenced by a nephritic flare as defined by renal criteria as noted in U.S. patent application publication 2006/0024295.

Lupus nephritis is optionally diagnosed and classified as ISN/WHO class I, class II, class III, class IV, class V, or class VI lupus nephritis, e.g., as set forth in Weening et al. (2004) “The classification of glomerulonephritis in systemic lupus erythematosus revisited” Kidney International 65:521-530.

Yet another aspect of the invention provides methods of treating multiple sclerosis (MS) by administration of a non-depleting CD4 antibody, optionally in combination with another compound used clinically or experimentally to treat MS. MS is an autoimmune demyelinating disorder that is believed to be T lymphocyte dependent. MS generally exhibits a relapsing-remitting course or a chronic progressive course. Relapsing-remitting MS (RRMS) is characterized by partial or total recovery after attacks. Secondary-progressive MS (SPMS) is a relapsing-remitting course which becomes steadily progressive. Attacks and partial recoveries may continue to occur. Primary-progressive MS (PPMS) is progressive from the onset. Symptoms in patients with PPMS generally do not remit—i.e., decrease in intensity.

Common signs and symptoms of MS include paresthesias in one or more extremities, in the trunk, or on one side of the face; weakness or clumsiness of a leg or hand; or visual disturbances (such as partial blindness and pain in one eye), dimness of vision, or scotomas. Other common early symptoms are ocular palsy resulting in double vision (diplopia), transient weakness of one or more extremities, slight stiffness or unusual fatigability of a limb, minor gait disturbances, difficulty with bladder control, vertigo, and mild emotional disturbances (Berkow et al. (ed.), 1999, Merck Manual of Diagnosis and Therapy: 17th Ed). The etiology of MS is unknown, however, viral infections, genetic predisposition, environment, and autoimmunity all appear to contribute to the disorder. Lesions in MS patients contain infiltrates of predominantly T lymphocyte mediated microglial cells and infiltrating macrophages. CD4+ T lymphocytes are the predominant cell type present at these lesions. The hallmark of the MS lesion is plaque, an area of demyelination sharply demarcated from the usual white matter seen in MRI scans. Histological appearance of MS plaques varies with different stages of the disease. In active lesions, the blood-brain barrier is damaged, thereby permitting extravasation of serum proteins into extracellular spaces. Inflammatory cells can be seen in perivascular cuffs and throughout white matter. CD4-T-cells, especially Th1, accumulate around postcapillary venules at the edge of the plaque and are also scattered in the white matter. In active lesions, up-regulation of adhesion molecules and markers of lymphocyte and monocyte activation, such as IL2-R and CD26 have also been observed. Demyelination in active lesions is not accompanied by destruction of oligodendrocytes. In contrast, during chronic phases of the disease, lesions are characterized by a loss of oligodendrocytes and hence, the presence of myelin oligodendrocyte glycoprotein (MOG) antibodies in the blood.

Yet another aspect of the invention provides methods of treating RA by administration of a non-depleting CD4 antibody, optionally in combination with another compound used clinically or experimentally to treat RA. In certain aspects, methods of treating RA by administration of a non-depleting CD4 antibody, optionally in combination with another compound used clinically or experimentally to treat RA, in patients who previously failed treatment with at least one biologic therapeutic compound are provided. Yet another aspect of the invention provides methods of treating RA by administration of a non-depleting CD4 antibody, optionally in combination with another compound used clinically or experimentally to treat RA, in patients who previously failed treatment with at least one disease-modifying antirheumatic drug (DMARD).

Rheumatoid arthritis (RA) is a chronic systemic autoimmune inflammatory disease that mainly involves the synovial membrane of multiple joints with resultant injury to the articular cartilage, resulting in joint destruction and ultimately in disability in most patients. (Lawrence et al., Arthritis Rheum. 41:778-99, 1998; Helmick et al., Arthritis Rheum. 58(1):15-25, 2008; Pincus et al., Arthritis Rheum. 27(8):864-72, 1984; Corbett et al., Br. J. Rheumatol. 32(8):717-23, 1993). The main presenting symptoms in RA are pain, stiffness, swelling, and loss of function (Bennett J C, The etiology of rheumatoid arthritis, In Textbook of Rheumatology [Kelley W N, Harris E D, Ruddy S, Sledge C B, eds.] W B Saunders, Philadelphia pp 879-886, 1985).

The pathogenesis is T lymphocyte dependent and is associated with the production of rheumatoid factors, auto-antibodies directed against self IgG, with the resultant formation of immune complexes that attain high levels in joint fluid and blood. These complexes in the joint may induce the marked infiltrate of lymphocytes and monocytes into the synovium and subsequent marked synovial changes; the joint space/fluid is infiltrated by similar cells with the addition of numerous neutrophils. Tissues affected are primarily the joints, often in symmetrical pattern. However, extra-articular disease also occurs in two major forms. One form is the development of extra-articular lesions with ongoing progressive joint disease and typical lesions of pulmonary fibrosis, vasculitis, and cutaneous ulcers. The second form of extra-articular disease is the so-called Felty's syndrome which occurs late in the RA disease course, sometimes after joint disease has become quiescent, and involves the presence of neutropenia, thrombocytopenia and splenomegaly. This can be accompanied by vasculitis in multiple organs with formations of infarcts, skin ulcers and gangrene. Patients often also develop rheumatoid nodules in the subcutis tissue overlying affected joints; the nodules late stage have necrotic centers surrounded by a mixed inflammatory cell infiltrate. Other manifestations which can occur in RA include: pericarditis, pleuritis, coronary arteritis, interstitial pneumonitis with pulmonary fibrosis, keratoconjunctivitis sicca, and rheumatoid nodules.

Juvenile chronic arthritis is a chronic idiopathic inflammatory disease which begins often at less than 16 years of age. Its phenotype has some similarities to RA; some patients which are rheumatoid factor positive are classified as juvenile rheumatoid arthritis. The disease is sub-classified into three major categories: pauciarticular, polyarticular, and systemic. The arthritis can be severe and is typically destructive and leads to joint ankylosis and retarded growth. Other manifestations can include chronic anterior uveitis and systemic amyloidosis.

Spondyloarthropathies are a group of disorders with some common clinical features and the common association with the expression of HLA-B27 gene product. Exemplary disorders include: ankylosing spondylitis, Reiter's syndrome (reactive arthritis), arthritis associated with inflammatory bowel disease, spondylitis associated with psoriasis, juvenile onset spondyloarthropathy and undifferentiated spondyloarthropathy. Distinguishing features include sacroileitis with or without spondylitis; inflammatory asymmetric arthritis; association with HLA-B27 (a serologically defined allele of the HLA-B locus of class I MHC); ocular inflammation, and absence of autoantibodies associated with other rheumatoid disease. The cell most implicated as key to induction of the disease is the CD8+ T lymphocyte, a cell which targets antigen presented by class I MHC molecules. CD8+ T cells may react against the class I MHC allele HLA-B27 as if it were a foreign peptide expressed by MHC class I molecules. It has been hypothesized that an epitope of HLA-B27 may mimic a bacterial or other microbial antigenic epitope and thus induce a CD8+ T cells response.

In certain instances, diagnosis of RA is made if a patient satisfies certain American College of Rheumatology Criteria (ACR). Criteria include morning stiffness in and around the joints lasting for at least 1 hour before maximal improvement; arthritis of three or more joint areas: at least three joint areas have simultaneously had soft tissue swelling or fluid (not bony overgrowth alone) observed by a physician; the 14 possible joint areas (right and left) are proximal interphalangeal (PIP), metacarpophalangeal (MCP), wrist, elbow, knee, ankle, and metatarsophalangeal (MTP) joints; arthritis of hand joints: at least one joint area swollen as above in wrist, MCP, or PIP joint; symmetric arthritis: simultaneous involvement of the same joint areas (as in arthritis of three or more joint areas, above) on both sides of the body (bilateral involvement of PIP, MCP, or MTP joints is acceptable without absolute symmetry); rheumatoid nodules: subcutaneous nodules over bony prominences or extensor surfaces or in juxta-articular regions that are observed by a physician; serum rheumatoid factor: demonstration of abnormal amounts of serum rheumatoid factor by any method that has been positive in fewer than five percent of normal control patients; radiographic changes: radiographic changes typical of rheumatoid arthritis on posteroanterior hand and wrist X-rays, which must include erosions or unequivocal bony decalcification localized to or most marked adjacent to the involved joints (osteoarthritis changes alone do not qualify). Diagnosis of RA is typically made if a patient satisfies at least four of the above criteria.

Initial therapy of RA typically involves administration of one or more of the following drugs: nonsteroidal antiinflammatory drugs (NSAIDs), glucocorticoid (via joint injection), and low-dose prednisone. See “Guidelines for the management of rheumatoid arthritis,” Arthritis & Rheumatism 46(2): 328-346 (February, 2002). The majority of patients with newly diagnosed RA are started with disease-modifying antirheumatic drug (DMARD) therapy within 3 months of diagnosis. DMARDs commonly used in RA are hydroxychloroquine, sulfasalazine, methotrexate (plus oral and subcutaneous methotrexate), leflunomide, azathioprine, D-penicillamine, Gold (oral), Gold (intramuscular), minocycline, cyclosporine, Staphylococcal protein A immunoadsorption.

In certain instances, TNFα inhibitors have been used for therapy of RA. Exemplary TNFα inhibitors include etanercept (sold under the trade name ENBREL®), infliximab (sold under the trade name REMICADE®), adalimumab (sold under the trade name HUMIRA®), golimumab (sold under the trade name SIMPONI™) and certolizumab pegol (sold under the trade name CIMZIA)®.

Etanercept (sold under the trade name ENBREL®) is an injectable drug approved in the U.S. for therapy of active RA. Etanercept binds to TNFα and serves to remove most TNFα from joints and blood, thereby preventing TNFα from promoting inflammation and other symptoms of rheumatoid arthritis. Etanercept is an “immunoadhesin” fusion protein consisting of the extracellular ligand binding portion of the human 75 kD (p75) tumor necrosis factor receptor (TNFR) linked to the Fc portion of a human IgG1. The drug has been associated with negative side effects including serious infections and sepsis, and nervous system disorders such as multiple sclerosis (MS). See, e.g., www.reniicade-infliximab.com/pages/enbrel_embrel.html.

Infliximab, sold under the trade name REMICADE®, is an immune-suppressing drug prescribed to treat RA and Crohn's disease. Infliximab is a chimeric monoclonal antibody that binds to TNFα and reduces inflammation in the body by targeting and binding to TNFα which produces inflammation. Infliximab has been linked to certain fatal reactions such as heart failure and infections including tuberculosis as well as demyelination resulting in MS. See, e.g., www.remicade-infliximab.com.

In 2002, Abbott Laboratories received FDA approval to market adalimumab (sold under the trade name HUMIRA®), previously known as D2E7. Adalimumab is a human monoclonal antibody that binds to TNFα and is approved for reducing the signs and symptoms and inhibiting the progression of structural damage in adults with moderately to severely active RA who have had insufficient response to one or more traditional disease modifying DMARDs.

In April 2009, Centocor Ortho Biotech Inc. received FDA approval to market golimumab (sold under the trade name SIMPONI™) for patients with moderate to severe RA, psoriatic arthritis, and ankylosing spondylitis. Golimumab is a human IgG1ε monoclonal antibody specific for human TNFα and which is self-administered by patients subcutaneously once every month. Golimumab binds to both soluble and transmembrane bioactive forms of TNFα. Similar to other agents that inhibit TNFα, golimumab has been associated with certain adverse events such as risk of infection, including serious and life-threatening fungal infections.

In May 2009, certolizumab pegol (sold under the trade name CIMZIA®) was approved by the FDA for treatment of patients with RA. It is administered by a healthcare professional by subcutaneous injection every two weeks during induction and then every four weeks during maintenance. Certolizumab pegol is a recombinant, humanized antibody Fab′ fragment, with specificity for human TNFα, conjugated to an approximately 40 kDa polyethylene glycol (PEG2MAL40K). Certolizumab pegol has also been associated with certain safety risks such as increased risk of serious infection, similar to other TNFα inhibitors.

In certain instances, the rituximab antibody (sold under the trade name RITUXAN®) has been used as a therapy for RA. Rituximab is a genetically engineered chimeric murine/human monoclonal antibody directed against the CD20 antigen. Rituximab is the antibody called “C2B8” in U.S. Pat. No. 5,736,137 issued Apr. 7, 1998 (Anderson et al.).

Another anti-CD20 antibody is ocrelizumab. Ocrelizumab is a humanized variant of an anti-CD20 antibody, 2H7. Such humanized 2H7 variants are described, for example, in International Publication No. WO 2004/056312 (International Application No. PCT/US2003/040426).

A further aspect of the invention provides methods of treating transplant recipients or subjects with autoimmune diseases such as asthma, psoriasis, inflammatory bowel disease (e.g., Crohn's disease and ulcerative colitis), and Sjogren's syndrome by administration of a non-depleting CD4 antibody, optionally in combination with another compound used clinically or experimentally to treat autoimmune disease.

A. CD4 Antibodies

A number of CD4 antibodies, both depleting and non-depleting, have been described. Use of such antibodies to induce tolerance to antigens, including autoantigens, has also been reported. See, e.g., U.S. Pat. No. 4,695,459; U.S. Pat. No. 6,056,956 to Cobbold and Waldmann entitled “Non-depleting anti-CD4 monoclonal antibodies and tolerance induction”; U.S. Pat. No. 5,690,933 to Cobbold and Waldmann entitled “Monoclonal antibodies for inducing tolerance”; European patent application publication 0240344 by Cobbold et al. entitled “Monoclonal antibodies and their use”; U.S. Pat. No. 6,136,310 to Hanna et al. entitled “Recombinant anti-CD4 antibodies for human therapy”; U.S. Pat. No. 5,756,096 to Newman et al. entitled “Recombinant antibodies for human therapy”; U.S. Pat. No. 5,750,105 to Newman et al. entitled “Recombinant antibodies for human therapy”; U.S. Pat. No. 4,381,295 to Kung and Goldstein entitled “Monoclonal antibody to human helper T cells and methods of preparing same”; Waldmann (1989) “Manipulation of T-cell responses with monoclonal antibodies” Ann Rev Immunol 7:407-44; and Wofsy and Seaman (1987) “Reversal of advanced murine lupus in NZB/NZW F1 mice by treatment with monoclonal antibody to L3T4” J Immunol 138:3247-53. In particular, a non-depleting CD4 antibody and its use in inducing tolerance has been described in U.S. patent application publication 2003/0108518 by Frewin et al. entitled “TRX1 antibody and uses therefor” and U.S. patent application publication 2003/0219403 by Frewin et al. entitled “Compositions and methods of tolerizing a primate to an antigen,” each of which is hereby incorporated by reference.

Exemplary non-depleting CD4 antibodies suitable for use in certain of the methods include, but are not limited to, the TRX1 antibodies described in U.S. patent application publication 2003/0108518 by Frewin et al. entitled “TRX1 antibody and uses therefor” and U.S. patent application publication 2003/0219403 by Frewin et al. entitled “Compositions and methods of tolerizing a primate to an antigen.” These antibodies are humanized antibodies including modified constant regions of a human antibody, light and heavy chain framework regions of a human antibody, and light and heavy chain CDRs derived from a mouse monoclonal antibody.

Additional exemplary, non-depleting CD4 antibodies suitable for use in certain of the methods include, but are not limited to, non-depleting CD4 antibodies modified to alter effector function, including, but not limited to, ADCC, CDC, and serum half-life. In certain such embodiments, modified non-depleting CD4 antibodies have the ability to bind FcRn with an increased binding relative to the unmodified antibody. In certain embodiments, modified non-depleting CD4 antibodies include a substitution at heavy-chain position 434, including, but not limited to, N434A and N434H. In certain embodiments, modified non-depleting CD4 antibodies include a substitution at heavy-chain position 297, including, but not limited to, N297A. In certain embodiments, non-depleting CD4 antibodies include a substitution at heavy-chain position 297 and a substitution of heavy-chain position 434.

In certain embodiments, the non-depleting CD4 antibody is any one of the antibodies as shown in Table 2 in Example 1. The antibody can have a light chain amino acid sequence set forth in SEQ ID NO:1 and a heavy chain amino acid sequence set forth in SEQ ID NO:3, a light chain amino acid sequence set forth in SEQ ID NO:1 and a heavy chain amino acid sequence set forth in SEQ ID NO:4, a light chain amino acid sequence set forth in SEQ ID NO:1 and a heavy chain amino acid sequence set forth in SEQ ID NO:5, or a light chain amino acid sequence set forth in SEQ ID NO:1 and a heavy chain amino acid sequence set forth in SEQ ID NO:6, a light chain amino acid sequence set forth in SEQ ID NO:2 and a heavy chain amino acid sequence set forth in SEQ ID NO:3, a light chain amino acid sequence set forth in SEQ ID NO:2 and a heavy chain amino acid sequence set forth in SEQ ID NO:4, a light chain amino acid sequence set forth in SEQ ID NO:2 and a heavy chain amino acid sequence set forth in SEQ ID NO:5, or a light chain amino acid sequence set forth in SEQ ID NO:2 and a heavy chain amino acid sequence set forth in SEQ ID NO:6. In a related class of embodiments, the antibody comprises a CD4 binding fragment of an antibody that comprises a light chain amino acid sequence set forth in SEQ ID NO:1 and a heavy chain amino acid sequence set forth in SEQ ID NO:3, a light chain amino acid sequence set forth in SEQ ID NO:1 and a heavy chain amino acid sequence set forth in SEQ ID NO:4, a light chain amino acid sequence set forth in SEQ ID NO:1 and a heavy chain amino acid sequence set forth in SEQ ID NO:5, or a light chain amino acid sequence set forth in SEQ ID NO:1 and a heavy chain amino acid sequence set forth in SEQ ID NO:6, a light chain amino acid sequence set forth in SEQ ID NO:2 and a heavy chain amino acid sequence set forth in SEQ ID NO:3, a light chain amino acid sequence set forth in SEQ ID NO:2 and a heavy chain amino acid sequence set forth in SEQ ID NO:4, a light chain amino acid sequence set forth in SEQ ID NO:2 and a heavy chain amino acid sequence set forth in SEQ ID NO:5, or a light chain amino acid sequence set forth in SEQ ID NO:2 and a heavy chain amino acid sequence set forth in SEQ ID NO:6.

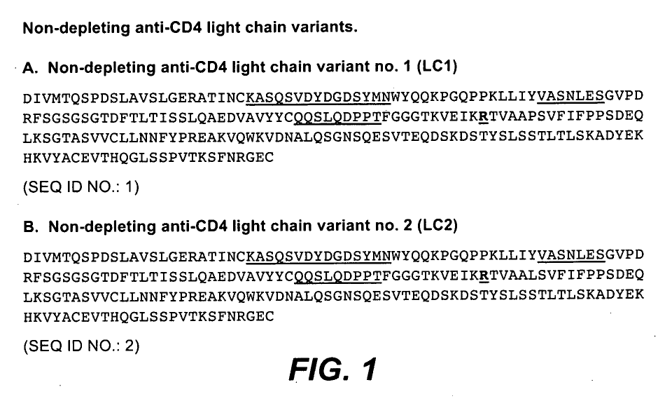

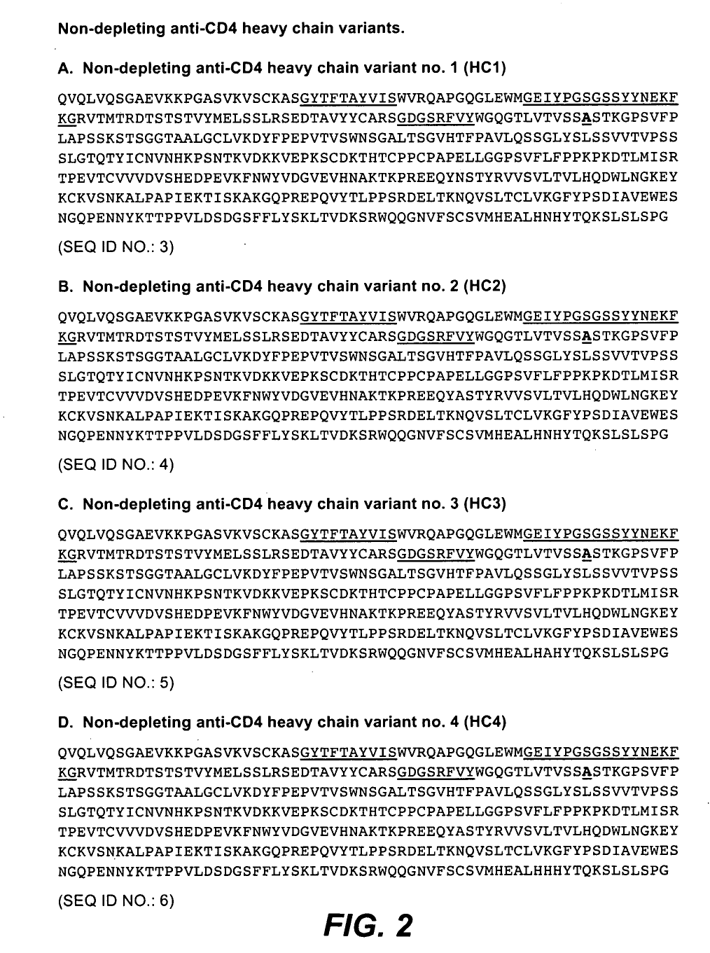

Antibodies comprising one or more CDRs from a non-depleting CD4 antibody are also useful in the methods. Thus, in one class of embodiments, the non-depleting CD4 antibody comprises CDR1 (SEQ ID NO.: 7), CDR2 (SEQ ID NO.: 8), or CDR3 (SEQ ID NO.: 9) of the light chain shown in FIGS. 1A and 1B. The antibody optionally includes CDR1, CDR2, and CDR3 of the light chain shown in FIGS. 1A and 1B (SEQ ID NOs.: 7-9). Similarly, in one class of embodiments, the antibody comprises CDR1 (SEQ ID NO.: 10), CDR2 (SEQ ID NO.: 11), or CDR3 (SEQ ID NO.: 12) of the heavy chain shown in FIGS. 2A-D. The antibody optionally includes CDR1, CDR2, and CDR3 of the heavy chain shown in FIGS. 2A-D (SEQ ID NOs.: 10-12). In one class of embodiments, the antibody comprises CDR1, CDR2, and CDR3 of the light chain shown in FIGS. 1A and 1B (SEQ ID NOs.: 7-9) and CDR1, CDR2, and CDR3 of the heavy chain shown in FIGS. 2A-D (SEQ ID NOs.: 10-12). The antibody optionally also includes FR1, FR2, FR3, and/or FR4 of the light chain shown in FIG. 1A or FIG. 1B and/or FR1, FR2, FR3, and/or FR4 of the heavy chain shown in FIG. 2A, FIG. 2B, FIG. 2C, or FIG. 2D.

Other exemplary antibodies include, but are not limited to, antibodies that bind the same epitope as a non-depleting CD4 antibody as described herein (e.g., as any one of an antibody shown Table 2 in Example 1).

In certain embodiments, the subject is a human and the antibody is a humanized or human antibody. It will be evident that for treatment of a non-human mammal, the antibody is optionally adapted for use in that animal, for example, by incorporation of framework and constant region sequences from an immunoglobulin from a mammal of the appropriate species. The antibody is optionally a monoclonal antibody, an intact antibody, an antibody fragment, and/or a native antibody.

The antibody optionally has a reduced effector function, e.g., as compared to wild-type human IgG1, such that its ability to induce complement activation and/or antibody dependent cell-mediated cytotoxicity is decreased. For example, in certain embodiments, the antibody has a reduced (or no) binding to a Fcγ receptor. Similarly, in certain embodiments, the antibody has an aglycosylated Fc portion. In certain embodiments, the antibody is a modified, or variant, non-depleting CD4 antibody having an increased binding to FcRn relative to the binding of the unmodified antibody to FcRn.

1. Antibody Fragments

The present invention encompasses antibody fragments. Antibody fragments may be generated by traditional means, such as enzymatic digestion, or by recombinant techniques. In certain circumstances there are advantages of using antibody fragments, rather than whole antibodies. The smaller size of the fragments allows for rapid clearance. For a review of certain antibody fragments, see Hudson et al. (2003) Nat. Med. 9:129-134.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10: 163-167 (1992)). According to another approach, F(ab′)₂ fragments can be isolated directly from recombinant host cell culture. Fab and F(ab′)₂ fragment with increased in vivo half-life comprising salvage receptor binding epitope residues are described in U.S. Pat. No. 5,869,046. Other techniques for the production of antibody fragments will be apparent to the skilled practitioner. In certain embodiments, an antibody is a single chain Fv fragment (scFv). See WO 93/16185; U.S. Pat. Nos. 5,571,894; and 5,587,458. Fv and scFv are the only species with intact combining sites that are devoid of constant regions; thus, they may be suitable for reduced nonspecific binding during in vivo use. scFv fusion proteins may be constructed to yield fusion of an effector protein at either the amino or the carboxy terminus of an scFv. See Antibody Engineering, ed. Borrebaeck, supra. The antibody fragment may also be a “linear antibody”, e.g., as described in U.S. Pat. No. 5,641,870, for example. Such linear antibodies may be monospecific or bispecific.

2. Humanized Antibodies

The invention encompasses humanized antibodies. Various methods for humanizing non-human antibodies are known in the art. For example, a humanized antibody can have one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al. (1986) Nature 321:522-525; Riechmann et al. (1988) Nature 332:323-327; Verhoeyen et al. (1988) Science 239:1534-1536), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies can be important to reduce antigenicity. According to the so-called “best-fit” method, the sequence of the variable domain of a rodent antibody is screened against the entire library of known human variable-domain sequences. The human sequence which is closest to that of the rodent is then accepted as the human framework for the humanized antibody. See, e.g., Sims et al. (1993) J. Immunol. 151:2296; Chothia et al. (1987) J. Mol. Biol. 196:901. Another method uses a particular framework derived from the consensus sequence of all human antibodies of a particular subgroup of light or heavy chains. The same framework may be used for several different humanized antibodies. See, e.g., Carter et al. (1992) Proc. Natl. Acad. Sci. USA, 89:4285; Presta et al. (1993) J. Immunol., 151:2623.

It is further generally desirable that antibodies be humanized with retention of high affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

3. Human Antibodies

Human antibodies of the invention can be constructed by combining Fv clone variable domain sequence(s) selected from human-derived phage display libraries with known human constant domain sequences(s) as described above. Alternatively, human monoclonal antibodies of the invention can be made by the hybridoma method. Human myeloma and mouse-human heteromyeloma cell lines for the production of human monoclonal antibodies have been described, for example, by Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).

It is now possible to produce transgenic animals (e.g. mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90: 2551 (1993); Jakobovits et al., Nature, 362: 255 (1993); Bruggermann et al., Year in Immunol., 7: 33 (1993).

Gene shuffling can also be used to derive human antibodies from non-human, e.g. rodent, antibodies, where the human antibody has similar affinities and specificities to the starting non-human antibody. According to this method, which is also called “epitope imprinting”, either the heavy or light chain variable region of a non-human antibody fragment obtained by phage display techniques as described herein is replaced with a repertoire of human V domain genes, creating a population of non-human chain/human chain scFv or Fab chimeras. Selection with antigen results in isolation of a non-human chain/human chain chimeric scFv or Fab wherein the human chain restores the antigen binding site destroyed upon removal of the corresponding non-human chain in the primary phage display clone, i.e. the epitope governs (imprints) the choice of the human chain partner. When the process is repeated in order to replace the remaining non-human chain, a human antibody is obtained (see PCT WO 93/06213 published Apr. 1, 1993). Unlike traditional humanization of non-human antibodies by CDR grafting, this technique provides completely human antibodies, which have no FR or CDR residues of non-human origin.

4. Antibody Variants

In some embodiments, amino acid sequence modification(s) of the antibodies described herein are contemplated. For example, it may be desirable to improve the binding affinity and/or other biological properties of the antibody. Amino acid sequence variants of the antibody may be prepared by introducing appropriate changes into the nucleotide sequence encoding the antibody, or by peptide synthesis. Such modifications include, for example, deletions from, and/or insertions into and/or substitutions of, residues within the amino acid sequences of the antibody. Any combination of deletion, insertion, and substitution can be made to arrive at the final construct, provided that the final construct possesses the desired characteristics. The amino acid alterations may be introduced in the subject antibody amino acid sequence at the time that sequence is made.

A useful method for identification of certain residues or regions of the antibody that are favored locations for mutagenesis is called “alanine scanning mutagenesis” as described by Cunningham and Wells (1989) Science, 244:1081-1085. Here, a residue or group of target residues are identified (e.g., charged residues such as arg, asp, his, lys, and glu) and replaced by a neutral or negatively charged amino acid (e.g., alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed immunoglobulins are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme or a polypeptide which increases the serum half-life of the antibody.

In certain embodiments, an antibody of the invention is altered to increase or decrease the extent to which the antibody is glycosylated. Glycosylation of polypeptides is typically either N-linked or O-linked. N-linked refers to the attachment of a carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition or deletion of glycosylation sites to the antibody is conveniently accomplished by altering the amino acid sequence such that one or more of the above-described tripeptide sequences (for N-linked glycosylation sites) is created or removed. The alteration may also be made by the addition, deletion, or substitution of one or more serine or threonine residues to the sequence of the original antibody (for O-linked glycosylation sites).

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. (1997) TIBTECH 15:26-32. The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

Where the antibody comprises an Fc region, the carbohydrate attached thereto may be altered or removed. For example, in one glycosylation variant herein, one or more amino acid substitutions are introduced in an Fc region of an antibody to eliminate one or more glycosylation sites. Such an aglycosylated antibody can have reduced effector function, e.g., as compared to wild-type human IgG1, such that its ability to induce complement activation and/or antibody dependent cell-mediated cytotoxicity is decreased, and the aglycosylated antibody can have reduced (or no) binding to a Fcγ receptor.

In certain embodiments, the invention contemplates an antibody variant that possesses some but not all effector functions, which make it a desirable candidate for many applications in which the half life of the antibody in vivo is important yet certain effector functions (such as complement and ADCC) are unnecessary or deleterious. In certain embodiments, the Fc activities of the antibody are measured to ensure that only the desired properties are maintained. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-92 (1991). Non-limiting examples of in vitro assays to assess ADCC activity of a molecule of interest is described in U.S. Pat. No. 5,500,362 (see, e.g. Hellstrom, I., et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CYTOTOX 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, for example, Petkova, S. B. et al., Int'l. Immunol. 18(12): 1759-1769 (2006)).

Other antibody variants having one or more amino acid substitutions are provided. Sites of interest for substitutional mutagenesis include the hypervariable regions, but FR alterations are also contemplated. Conservative substitutions are shown in Table 1 under the heading of “conservative substitutions.” More substantial changes, denominated “exemplary substitutions” are provided in Table 1, or as further described below in reference to amino acid classes. Amino acid substitutions may be introduced into an antibody of interest and the products screened, e.g., for a desired activity, such as improved antigen binding, decreased immunogenicity, improved ADCC or CDC, etc.

TABLE 1

| Original | Exemplary | Conservative |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; | Leu |
| | Phe; Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala; Norleucine | |

Modifications in the biological properties of an antibody may be accomplished by selecting substitutions that affect (a) the structure of the polypeptide backbone in the area of the substitution, for example, as a sheet or helical conformation, (b) the charge or hydrophobicity of the molecule at the target site, or (c) the bulk of the side chain. Amino acids may be grouped according to similarities in the properties of their side chains (in A. L. Lehninger, in Biochemistry, second ed., pp. 73-75, Worth Publishers, New York (1975)):

(1) non-polar: Ala (A), Val (V), Leu (L), Ile (I), Pro (P), Phe (F), Trp (W), Met (M)

(2) uncharged polar: Gly (G), Ser (S), Thr (T), Cys (C), Tyr (Y), Asn (N), Gln (O)

(3) acidic: Asp (D), Glu (E)

(4) basic: Lys (K), Arg (R), His(H)

Alternatively, naturally occurring residues may be divided into groups based on common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: H is, Lys, Arg;

(5) residues that influence chain orientation: Gly, Pro;

(6) aromatic: Trp, Tyr, Phe.

Non-conservative substitutions will entail exchanging a member of one of these classes for another class. Such substituted residues also may be introduced into the conservative substitution sites or, into the remaining (non-conserved) sites.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

One type of substitutional variant involves substituting one or more hypervariable region residues of a parent antibody (e.g. a humanized or human antibody). Generally, the resulting variant(s) selected for further development will have modified (e.g., improved) biological properties relative to the parent antibody from which they are generated. An exemplary substitutional variant is an affinity matured antibody, which may be conveniently generated using phage display-based affinity maturation techniques. Briefly, several hypervariable region sites (e.g. 6-7 sites) are mutated to generate all possible amino acid substitutions at each site. The antibodies thus generated are displayed from filamentous phage particles as fusions to at least part of a phage coat protein (e.g., the gene III product of M13) packaged within each particle. The phage-displayed variants are then screened for their biological activity (e.g. binding affinity). In order to identify candidate hypervariable region sites for modification, scanning mutagenesis (e.g., alanine scanning) can be performed to identify hypervariable region residues contributing significantly to antigen binding. Alternatively, or additionally, it may be beneficial to analyze a crystal structure of the antigen-antibody complex to identify contact points between the antibody and antigen. Such contact residues and neighboring residues are candidates for substitution according to techniques known in the art, including those elaborated herein. Once such variants are generated, the panel of variants is subjected to screening using techniques known in the art, including those described herein, and variants with superior properties in one or more relevant assays may be selected for further development.

Antibodies with altered C1q binding and/or CDC are described in WO 1999/51642 and U.S. Pat. Nos. 6,194,551, 6,242,195, 6,528,624, and 6,538,124 (Idusogie et al.). The antibodies comprise an amino acid substitution at one or more of amino acid positions 270, 322, 326, 327, 329, 313, 333, and/or 334 of the Fc region thereof. Non-depleting anti-CD4 antibodies comprising such amino acid substitutions constitute an embodiment of the invention.

To increase the serum half-life of the antibody, one may incorporate a salvage receptor binding epitope into the antibody (or an antibody fragment) as described in U.S. Pat. No. 5,739,277, for example. As used herein, the term salvage receptor binding epitope refers to an epitope of the Fc region of an IgG molecule (e.g., IgG₁, IgG₂, IgG₃, or IgG₄) that is responsible for increasing the in vivo serum half-life of the IgG molecule. Antibodies with substitutions in an Fc region thereof and increased serum half-lives are also described in WO 2000/42072 (Presta, L.). Non-depleting anti-CD4 antibodies comprising such a salvage receptor binding epitope constitute an embodiment of the invention.

Any of the non-depleting antibodies of the invention may comprise at least one substitution in the Fc region that improves FcRn binding or serum half-life, e.g., a non-depleting anti-CD4 variant antibody. For example, the invention further provides an antibody comprising a variant Fc region with altered neonatal Fc receptor (FcRn) binding affinity, for example, increased binding affinity for FcRn or increased binding to FcRn. FcRn is structurally similar to major histocompatibility complex (MHC) and consists of an α-chain noncovalently bound to β2-microglobulin. The multiple functions of the neonatal Fc receptor FcRn are reviewed in Ghetie and Ward (2000) Annu. Rev. Immunol. 18:39-766. FcRn plays a role in the passive delivery of immunoglobulin IgGs from mother to young and the regulation of serum IgG levels. FcRn acts as a salvage receptor, binding and transporting pinocytosed IgGs in intact form both within and across cells, and rescuing them from a default degradative pathway. Although the mechanisms responsible for salvaging IgGs are still unclear, it is thought that unbound IgGs are directed toward proteolysis in lysosomes, whereas bound IgGs are recycled to the surface of the cells and released. This control takes place within the endothelial cells located throughout adult tissues. FcRn is expressed in at least the liver, mammary gland, and adult intestine. FcRn binds to IgG; the FcRn-IgG interaction has been studied extensively and appears to involve residues at the CH2, CH3 domain interface of the Fc region of IgG. These residues interact with residues primarily located in the α2 domain of FcRn.

In certain embodiments of the invention, a non-depleting anti-CD4 variant antibody may display increased binding to FcRn and comprise an amino acid modification at any one or more of amino acid positions 238, 256, 265, 272, 286, 303, 305, 307, 311, 312, 317, 340, 356, 360, 362, 376, 378, 380, 382, 413, 424 or 434 of the Fc region, wherein the numbering of the residues in the Fc region is that of the EU index as in Kabat. See, e.g., U.S. Pat. No. 6,737,056; and, Shields et al., J. Biol. Chem. 276: 6591-6604 (2001). In one embodiment of the invention, an antibody comprises a variant IgG Fc region comprising at least an amino acid substitution at Asn 434 to His (N434H). In one embodiment of the invention, an antibody comprises a variant IgG Fc region comprising at least an amino acid substitution at Asn 434 to Ala (N434A). Typically, these variants comprise a higher binding affinity for FcRn or display increased binding to FcRn than polypeptides having native sequence/wild type sequence Fc region. These Fc variant polypeptide and antibodies have the advantage of being salvaged and recycled rather than degraded. These non-depleting anti-CD4 variant antibodies can be used in the methods provided herein. As just one example of a non-depleting CD4 variant antibody, any of the non-depleting anti-CD4 antibodies described herein can include a substitution at heavy-chain position 434, such as N434A or N434H.

Serum half-life of the antibody may also be increased by incorporation of a serum albumin binding peptide into the antibody as disclosed in U.S. Patent Publication No. 20040001827 (Dennis, M.). Non-depleting anti-CD4 antibodies comprising such serum albumin binding peptides constitute an embodiment of the invention.

It may be desirable to introduce one or more amino acid modifications in an Fc region of antibodies of the invention, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions including that of a hinge cysteine.

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

5. Antibody Derivatives

The antibodies of the present invention can be further modified to contain additional nonproteinaceous moieties that are known in the art and readily available. In certain embodiments, the moieties suitable for derivatization of the antibody are water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

B. Certain Methods of Making Antibodies

1. Certain Hybridoma-Based Methods

Monoclonal antibodies of the invention can be made using the hybridoma method first described by Kohler et al., Nature, 256:495 (1975), and further described, e.g., in Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981), and Ni, Xiandai Mianyixue, 26(4):265-268 (2006) regarding human-human hybridomas. Additional methods include those described, for example, in U.S. Pat. No. 7,189,826 regarding production of monoclonal human natural IgM antibodies from hybridoma cell lines. Human hybridoma technology (Trioma technology) is described in Vollmers and Brandlein, Histology and Histopathology, 20(3):927-937 (2005) and Vollmers and Brandlein, Methods and Findings in Experimental and Clinical Pharmacology, 27(3):185-91 (2005).

For various other hybridoma techniques, see, e.g., US 2006/258841; US 2006/183887 (fully human antibodies), US 2006/059575; US 2005/287149; US 2005/100546; US 2005/026229; and U.S. Pat. Nos. 7,078,492 and 7,153,507. An exemplary protocol for producing monoclonal antibodies using the hybridoma method is described as follows. In one embodiment, a mouse or other appropriate host animal, such as a hamster, is immunized to elicit lymphocytes that produce or are capable of producing antibodies that will specifically bind to the protein used for immunization. Antibodies are raised in animals by multiple subcutaneous (sc) or intraperitoneal (ip) injections of a polypeptide comprising CD4 or a fragment thereof, and an adjuvant, such as monophosphoryl lipid A (MPL)/trehalose dicrynomycolate (TDM) (Ribi Immunochem. Research, Inc., Hamilton, Mont.). A polypeptide comprising CD4 or a fragment thereof may be prepared using methods well known in the art, such as recombinant methods, some of which are further described herein. Serum from immunized animals is assayed for anti-CD4 antibodies, and booster immunizations are optionally administered. Lymphocytes from animals producing anti-CD4 antibodies are isolated. Alternatively, lymphocytes may be immunized in vitro.

Lymphocytes are then fused with myeloma cells using a suitable fusing agent, such as polyethylene glycol, to form a hybridoma cell. See, e.g., Goding, Monoclonal Antibodies: Principles and Practice, pp. 59-103 (Academic Press, 1986). Myeloma cells may be used that fuse efficiently, support stable high-level production of antibody by the selected antibody-producing cells, and are sensitive to a medium such as HAT medium. Exemplary myeloma cells include, but are not limited to, murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

The hybridoma cells thus prepared are seeded and grown in a suitable culture medium, e.g., a medium that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells. In certain embodiments, serum-free hybridoma cell culture methods are used to reduce use of animal-derived serum such as fetal bovine serum, as described, for example, in Even et al., Trends in Biotechnology, 24(3), 105-108 (2006).

Oligopeptides as tools for improving productivity of hybridoma cell cultures are described in Franek, Trends in Monoclonal Antibody Research, 111-122 (2005). Specifically, standard culture media are enriched with certain amino acids (alanine, serine, asparagine, proline), or with protein hydrolyzate fractions, and apoptosis may be significantly suppressed by synthetic oligopeptides, constituted of three to six amino acid residues. The peptides are present at millimolar or higher concentrations.

Culture medium in which hybridoma cells are growing may be assayed for production of monoclonal antibodies that bind to CD4. The binding specificity of monoclonal antibodies produced by hybridoma cells may be determined by immunoprecipitation or by an in vitro binding assay, such as radioimmunoassay (RIA) or enzyme-linked immunoadsorbent assay (ELISA). The binding affinity of the monoclonal antibody can be determined, for example, by Scatchard analysis. See, e.g., Munson et al., Anal. Biochem., 107:220 (1980).

After hybridoma cells are identified that produce antibodies of the desired specificity, affinity, and/or activity, the clones may be subcloned by limiting dilution procedures and grown by standard methods. See, e.g., Goding, supra. Suitable culture media for this purpose include, for example, D-MEM or RPMI-1640 medium. In addition, hybridoma cells may be grown in vivo as ascites tumors in an animal. Monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography. One procedure for isolation of proteins from hybridoma cells is described in US 2005/176122 and U.S. Pat. No. 6,919,436. The method includes using minimal salts, such as lyotropic salts, in the binding process and also using small amounts of organic solvents in the elution process.

2. Certain Library Screening Methods

Antibodies of the invention can be made by using combinatorial libraries to screen for antibodies with the desired activity or activities. For example, a variety of methods are known in the art for generating phage display libraries and screening such libraries for antibodies possessing the desired binding characteristics. Such methods are described generally in Hoogenboom et al. in Methods in Molecular Biology 178:1-37 (O'Brien et al., ed., Human Press, Totowa, N.J., 2001). For example, one method of generating antibodies of interest is through the use of a phage antibody library as described in Lee et al., J. Mol. Biol. (2004), 340(5):1073-93.

In principle, synthetic antibody clones are selected by screening phage libraries containing phage that display various fragments of antibody variable region (Fv) fused to phage coat protein. Such phage libraries are panned by affinity chromatography against the desired antigen. Clones expressing Fv fragments capable of binding to the desired antigen are adsorbed to the antigen and thus separated from the non-binding clones in the library. The binding clones are then eluted from the antigen, and can be further enriched by additional cycles of antigen adsorption/elution. Any of the antibodies of the invention can be obtained by designing a suitable antigen screening procedure to select for the phage clone of interest followed by construction of a full length antibody clone using the Fv sequences from the phage clone of interest and suitable constant region (Fc) sequences described in Kabat et al., Sequences of Proteins of Immunological Interest, Fifth Edition, NIH Publication 91-3242, Bethesda Md. (1991), vols. 1-3.

In certain embodiments, the antigen-binding domain of an antibody is formed from two variable (V) regions of about 110 amino acids, one each from the light (VL) and heavy (VH) chains, that both present three hypervariable loops (HVRs) or complementarity-determining regions (CDRs). Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described in Winter et al., Ann. Rev. Immunol., 12: 433-455 (1994). As used herein, scFv encoding phage clones and Fab encoding phage clones are collectively referred to as “Fv phage clones” or “Fv clones.”

Repertoires of VH and VL genes can be separately cloned by polymerase chain reaction (PCR) and recombined randomly in phage libraries, which can then be searched for antigen-binding clones as described in Winter et al., Ann. Rev. Immunol., 12: 433-455 (1994). Libraries from immunized sources provide high-affinity antibodies to the immunogen without the requirement of constructing hybridomas. Alternatively, the naive repertoire can be cloned to provide a single source of human antibodies to a wide range of non-self and also self antigens without any immunization as described by Griffiths et al., EMBO J, 12: 725-734 (1993). Finally, naive libraries can also be made synthetically by cloning the unrearranged V-gene segments from stem cells, and using PCR primers containing random sequence to encode the highly variable CDR3 regions and to accomplish rearrangement in vitro as described by Hoogenboom and Winter, J. Mol. Biol., 227: 381-388 (1992).

In certain embodiments, filamentous phage is used to display antibody fragments by fusion to the minor coat protein pIII. The antibody fragments can be displayed as single chain Fv fragments, in which VH and VL domains are connected on the same polypeptide chain by a flexible polypeptide spacer, e.g. as described by Marks et al., J. Mol. Biol., 222: 581-597 (1991), or as Fab fragments, in which one chain is fused to pIII and the other is secreted into the bacterial host cell periplasm where assembly of a Fab-coat protein structure which becomes displayed on the phage surface by displacing some of the wild type coat proteins, e.g. as described in Hoogenboom et al., Nucl. Acids Res., 19: 4133-4137 (1991).

In general, nucleic acids encoding antibody gene fragments are obtained from immune cells harvested from humans or animals. If a library biased in favor of anti-CD4 clones is desired, the subject is immunized with CD4 to generate an antibody response, and spleen cells and/or circulating B cells other peripheral blood lymphocytes (PBLs) are recovered for library construction. In a particular embodiment, a human antibody gene fragment library biased in favor of anti-CD4 clones is obtained by generating an anti-CD4 antibody response in transgenic mice carrying a functional human immunoglobulin gene array (and lacking a functional endogenous antibody production system) such that CD4 immunization gives rise to B cells producing human antibodies against CD4. The generation of human antibody-producing transgenic mice is described below.

Additional enrichment for anti-CD4 reactive cell populations can be obtained by using a suitable screening procedure to isolate B cells expressing CD4-specific membrane bound antibody, e.g., by cell separation using CD4 affinity chromatography or adsorption of cells to fluorochrome-labeled CD4 followed by flow-activated cell sorting (FACS).

Alternatively, the use of spleen cells and/or B cells or other PBLs from an unimmunized donor provides a better representation of the possible antibody repertoire, and also permits the construction of an antibody library using any animal (human or non-human) species in which CD4 is not antigenic. For libraries incorporating in vitro antibody gene construction, stem cells are harvested from the subject to provide nucleic acids encoding unrearranged antibody gene segments. The immune cells of interest can be obtained from a variety of animal species, such as human, mouse, rat, lagomorpha, luprine, canine, feline, porcine, bovine, equine, and avian species, etc.

Nucleic acid encoding antibody variable gene segments (including VH and VL segments) are recovered from the cells of interest and amplified. In the case of rearranged VH and VL gene libraries, the desired DNA can be obtained by isolating genomic DNA or mRNA from lymphocytes followed by polymerase chain reaction (PCR) with primers matching the 5′ and 3′ ends of rearranged VH and VL genes as described in Orlandi et al., Proc. Natl. Acad. Sci. (USA), 86: 3833-3837 (1989), thereby making diverse V gene repertoires for expression. The V genes can be amplified from cDNA and genomic DNA, with back primers at the 5′ end of the exon encoding the mature V-domain and forward primers based within the J-segment as described in Orlandi et al. (1989) and in Ward et al., Nature, 341: 544-546 (1989). However, for amplifying from cDNA, back primers can also be based in the leader exon as described in Jones et al., Biotechnol., 9: 88-89 (1991), and forward primers within the constant region as described in Sastry et al., Proc. Natl. Acad. Sci. (USA), 86: 5728-5732 (1989). To maximize complementarity, degeneracy can be incorporated in the primers as described in Orlandi et al. (1989) or Sastry et al. (1989). In certain embodiments, library diversity is maximized by using PCR primers targeted to each V-gene family in order to amplify all available VH and VL arrangements present in the immune cell nucleic acid sample, e.g. as described in the method of Marks et al., J. Mol. Biol., 222: 581-597 (1991) or as described in the method of Orum et al., Nucleic Acids Res., 21: 4491-4498 (1993). For cloning of the amplified DNA into expression vectors, rare restriction sites can be introduced within the PCR primer as a tag at one end as described in Orlandi et al. (1989), or by further PCR amplification with a tagged primer as described in Clackson et al., Nature, 352: 624-628 (1991).

Repertoires of synthetically rearranged V genes can be derived in vitro from V gene segments. Most of the human VH-gene segments have been cloned and sequenced (reported in Tomlinson et al., J. Mol. Biol., 227: 776-798 (1992)), and mapped (reported in Matsuda et al., Nature Genet., 3: 88-94 (1993); these cloned segments (including all the major conformations of the H1 and H2 loop) can be used to generate diverse VH gene repertoires with PCR primers encoding H3 loops of diverse sequence and length as described in Hoogenboom and Winter, J. Mol. Biol., 227: 381-388 (1992). VH repertoires can also be made with all the sequence diversity focused in a long H3 loop of a single length as described in Barbas et al., Proc. Natl. Acad. Sci. USA, 89: 4457-4461 (1992). Human Vε and Vλ segments have been cloned and sequenced (reported in Williams and Winter, Eur. J. Immunol., 23: 1456-1461 (1993)) and can be used to make synthetic light chain repertoires. Synthetic V gene repertoires, based on a range of VH and VL folds, and L3 and H3 lengths, will encode antibodies of considerable structural diversity. Following amplification of V-gene encoding DNAs, germline V-gene segments can be rearranged in vitro according to the methods of Hoogenboom and Winter, J. Mol. Biol., 227: 381-388 (1992).

Repertoires of antibody fragments can be constructed by combining VH and VL gene repertoires together in several ways. Each repertoire can be created in different vectors, and the vectors recombined in vitro, e.g., as described in Hogrefe et al., Gene, 128: 119-126 (1993), or in vivo by combinatorial infection, e.g., the 1oxP system described in Waterhouse et al., Nucl. Acids Res., 21: 2265-2266 (1993). The in vivo recombination approach exploits the two-chain nature of Fab fragments to overcome the limit on library size imposed by E. coli transformation efficiency. Naive VH and VL repertoires are cloned separately, one into a phagemid and the other into a phage vector. The two libraries are then combined by phage infection of phagemid-containing bacteria so that each cell contains a different combination and the library size is limited only by the number of cells present (about 10¹² clones). Both vectors contain in vivo recombination signals so that the VH and VL genes are recombined onto a single replicon and are co-packaged into phage virions. These huge libraries provide large numbers of diverse antibodies of good affinity (K_{d}⁻¹ of about 10⁻⁸ M).

Alternatively, the repertoires may be cloned sequentially into the same vector, e.g. as described in Barbas et al., Proc. Natl. Acad. Sci. USA, 88: 7978-7982 (1991), or assembled together by PCR and then cloned, e.g. as described in Clackson et al., Nature, 352: 624-628 (1991). PCR assembly can also be used to join VH and VL DNAs with DNA encoding a flexible peptide spacer to form single chain Fv (scFv) repertoires. In yet another technique, “in cell PCR assembly” is used to combine VH and VL genes within lymphocytes by PCR and then clone repertoires of linked genes as described in Embleton et al., Nucl. Acids Res., 20: 3831-3837 (1992).

The antibodies produced by naive libraries (either natural or synthetic) can be of moderate affinity (K_{d}⁻¹ of about 10⁶ to 10⁷ M⁻¹), but affinity maturation can also be mimicked in vitro by constructing and reselecting from secondary libraries as described in Winter et al. (1994), supra. For example, mutation can be introduced at random in vitro by using error-prone polymerase (reported in Leung et al., Technique, 1: 11-15 (1989)) in the method of Hawkins et al., J. Mol. Biol., 226: 889-896 (1992) or in the method of Gram et al., Proc. Natl. Acad. Sci. USA, 89: 3576-3580 (1992). Additionally, affinity maturation can be performed by randomly mutating one or more CDRs, e.g. using PCR with primers carrying random sequence spanning the CDR of interest, in selected individual Fv clones and screening for higher affinity clones. WO 9607754 (published 14 Mar. 1996) described a method for inducing mutagenesis in a complementarity determining region of an immunoglobulin light chain to create a library of light chain genes. Another effective approach is to recombine the VH or VL domains selected by phage display with repertoires of naturally occurring V domain variants obtained from unimmunized donors and screen for higher affinity in several rounds of chain reshuffling as described in Marks et al., Biotechnol., 10: 779-783 (1992). This technique allows the production of antibodies and antibody fragments with affinities of about 10⁻⁹ M or less.

Screening of the libraries can be accomplished by various techniques known in the art. For example, CD4 can be used to coat the wells of adsorption plates, expressed on host cells affixed to adsorption plates or used in cell sorting, or conjugated to biotin for capture with streptavidin-coated beads, or used in any other method for panning phage display libraries.

The phage library samples are contacted with immobilized CD4 under conditions suitable for binding at least a portion of the phage particles with the adsorbent. Normally, the conditions, including pH, ionic strength, temperature and the like are selected to mimic physiological conditions. The phages bound to the solid phase are washed and then eluted by acid, e.g. as described in Barbas et al., Proc. Natl. Acad. Sci. USA, 88: 7978-7982 (1991), or by alkali, e.g. as described in Marks et al., J. Mol. Biol., 222: 581-597 (1991), or by CD4 antigen competition, e.g. in a procedure similar to the antigen competition method of Clackson et al., Nature, 352: 624-628 (1991). Phages can be enriched 20-1,000-fold in a single round of selection. Moreover, the enriched phages can be grown in bacterial culture and subjected to further rounds of selection.

The efficiency of selection depends on many factors, including the kinetics of dissociation during washing, and whether multiple antibody fragments on a single phage can simultaneously engage with antigen. Antibodies with fast dissociation kinetics (and weak binding affinities) can be retained by use of short washes, multivalent phage display and high coating density of antigen in solid phase. The high density not only stabilizes the phage through multivalent interactions, but favors rebinding of phage that has dissociated. The selection of antibodies with slow dissociation kinetics (and good binding affinities) can be promoted by use of long washes and monovalent phage display as described in Bass et al., Proteins, 8: 309-314 (1990) and in WO 92/09690, and a low coating density of antigen as described in Marks et al., Biotechnol., 10: 779-783 (1992).

It is possible to select between phage antibodies of different affinities, even with affinities that differ slightly, for CD4. However, random mutation of a selected antibody (e.g. as performed in some affinity maturation techniques) is likely to give rise to many mutants, most binding to antigen, and a few with higher affinity. With limiting CD4, rare high affinity phage could be competed out. To retain all higher affinity mutants, phages can be incubated with excess biotinylated CD4, but with the biotinylated CD4 at a concentration of lower molarity than the target molar affinity constant for CD4. The high affinity-binding phages can then be captured by streptavidin-coated paramagnetic beads. Such “equilibrium capture” allows the antibodies to be selected according to their affinities of binding, with sensitivity that permits isolation of mutant clones with as little as two-fold higher affinity from a great excess of phages with lower affinity. Conditions used in washing phages bound to a solid phase can also be manipulated to discriminate on the basis of dissociation kinetics.

Anti-CD4 clones may be selected based on activity. In certain embodiments, the invention provides anti-CD4 antibodies that bind to living cells that naturally express CD4. In one embodiment, the invention provides anti-CD4 antibodies that block the binding between a CD4 ligand and CD4, but do not block the binding between a CD4 ligand and a second protein. Fv clones corresponding to such anti-CD4 antibodies can be selected by (1) isolating anti-CD4 clones from a phage library as described above, and optionally amplifying the isolated population of phage clones by growing up the population in a suitable bacterial host; (2) selecting CD4 and a second protein against which blocking and non-blocking activity, respectively, is desired; (3) adsorbing the anti-CD4 phage clones to immobilized CD4; (4) using an excess of the second protein to elute any undesired clones that recognize CD4-binding determinants which overlap or are shared with the binding determinants of the second protein; and (5) eluting the clones which remain adsorbed following step (4). Optionally, clones with the desired blocking/non-blocking properties can be further enriched by repeating the selection procedures described herein one or more times.

DNA encoding hybridoma-derived monoclonal antibodies or phage display Fv clones of the invention is readily isolated and sequenced using conventional procedures (e.g. by using oligonucleotide primers designed to specifically amplify the heavy and light chain coding regions of interest from hybridoma or phage DNA template). Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of the desired monoclonal antibodies in the recombinant host cells. Review articles on recombinant expression in bacteria of antibody-encoding DNA include Skerra et al., Curr. Opinion in Immunol., 5: 256 (1993) and Pluckthun, Immunol. Revs, 130: 151 (1992).

DNA encoding the Fv clones of the invention can be combined with known DNA sequences encoding heavy chain and/or light chain constant regions (e.g. the appropriate DNA sequences can be obtained from Kabat et al., supra) to form clones encoding full or partial length heavy and/or light chains. It will be appreciated that constant regions of any isotype can be used for this purpose, including IgG, IgM, IgA, IgD, and IgE constant regions, and that such constant regions can be obtained from any human or animal species. An Fv clone derived from the variable domain DNA of one animal (such as human) species and then fused to constant region DNA of another animal species to form coding sequence(s) for “hybrid,” full length heavy chain and/or light chain is included in the definition of “chimeric” and “hybrid” antibody as used herein. In certain embodiments, an Fv clone derived from human variable DNA is fused to human constant region DNA to form coding sequence(s) for full- or partial-length human heavy and/or light chains.

DNA encoding anti-CD4 antibody derived from a hybridoma of the invention can also be modified, for example, by substituting the coding sequence for human heavy- and light-chain constant domains in place of homologous murine sequences derived from the hybridoma clone (e.g. as in the method of Morrison et al., Proc. Natl. Acad. Sci. USA, 81: 6851-6855 (1984)). DNA encoding a hybridoma- or Fv clone-derived antibody or fragment can be further modified by covalently joining to the immunoglobulin coding sequence all or part of the coding sequence for a non-immunoglobulin polypeptide. In this manner, “chimeric” or “hybrid” antibodies are prepared that have the binding specificity of the Fv clone or hybridoma clone-derived antibodies of the invention.

3. Vectors, Host Cells, and Recombinant Methods

In practicing the present invention, many conventional techniques in molecular biology, microbiology, and recombinant DNA technology are optionally used. Such conventional techniques relate to vectors, host cells and recombinant methods. These techniques are well known and are explained in, for example, Berger and Kimmel, Guide to Molecular Cloning Techniques, Methods in Enzymology volume 152 Academic Press, Inc., San Diego, Calif.; Sambrook et al., Molecular Cloning-A Laboratory Manual (3rd Ed.), Vol. 1-3, Cold Spring Harbor Laboratory, Cold Spring Harbor, N.Y., 2000 and Current Protocols in Molecular Biology, F. M. Ausubel et al., eds., Current Protocols, a joint venture between Greene Publishing Associates, Inc. and John Wiley & Sons, Inc., (supplemented through 2006). Other useful references, e.g. for cell isolation and culture (e.g., for subsequent nucleic acid or protein isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (Eds.) (1995) Plant Cell, Tissue and Organ Culture; Fundamental Methods Springer Lab Manual, Springer-Verlag (Berlin Heidelberg New York) and Atlas and Parks (Eds.) The Handbook of Microbiological Media (1993) CRC Press, Boca Raton, Fla. Methods of making nucleic acids (e.g., by in vitro amplification, purification from cells, or chemical synthesis), methods for manipulating nucleic acids (e.g., site-directed mutagenesis, by restriction enzyme digestion, ligation, etc.), and various vectors, cell lines and the like useful in manipulating and making nucleic acids are described in the above references. In addition, essentially any polynucleotide (including, e.g., labeled or biotinylated polynucleotides) can be custom or standard ordered from any of a variety of commercial sources.

C. Administration

The physician administering treatment will be able to determine the appropriate dose for the individual subject. Preparation and dosing schedules for commercially available second therapeutic and other compounds administered in combination with the non-depleting CD4 antibodies may be used according to manufacturers' instructions or determined empirically by the skilled practitioner.

For the prevention or treatment of disease, the appropriate dosage of the non-depleting anti-CD4 antibody and any second therapeutic or other compound administered in combination with the non-depleting antibody will depend on the type of autoimmune disease to be treated, e.g., RA, SLE, MS, the severity and course of the disease, whether the non-depleting antibody or combination is administered for preventive or therapeutic purposes, previous therapy, the patient's clinical history and response to the antibody or combination, and the discretion of the attending physician. The non-depleting CD4 antibody or combination is suitably administered to the patient at one time or more typically over a series of treatments. In certain embodiments, the non-depleting CD4 antibody is administered once every week for a period of 8 weeks, or 6 months, or 1 year, or 2 years, or chronically for the lifetime of the patient. In certain embodiments, the treatment is self-administered by the patient.

Depending on the type and severity of the disease, about 1 μg/kg to 50 mg/kg (e.g. 0.1-20 mg/kg) of non-depleting CD4 antibody is an initial candidate dosage for administration to the patient, whether, for example, by one or more separate administrations, or by continuous infusion. In certain instances, a typical daily dosage might range from about 1 μg/kg to about 100 mg/kg or more, depending on the factors mentioned above. For repeated administrations over several days or longer, depending on the condition, the treatment is sustained until a desired suppression of disease symptoms occurs. However, other dosage regimens may be useful. Typically, the clinician will administer an antibody (alone or in combination with a second compound) of the invention until a dosage(s) is reached that provides the required biological effect. The progress of the therapy of the invention is easily monitored by conventional techniques and assays. For example, a non-depleting anti-CD4 antibody is optionally administered as described above or in U.S. Patent Publication No. 2003/0108518 or U.S. Patent Publication No. 2003/0219403.

In certain embodiments, between 0.2 and 10 mg/kg, or between 0.3 and 7.0 mg/kg, or between 1.0 and 5.0 mg/kg of a non-depleting anti-CD4 antibody is administered to a subject in need of treatment. In certain such embodiments, the dose administered is 0.3 mg/kg, 1.0 mg/kg, 2.0 mg/kg, 2.5 mg/kg, 3.0 mg/kg, 3.5 mg/kg, or 7.0 mg/kg. In certain embodiments, a flat dose between 150 mg and 350 mg, or between 200 mg and 300 mg, or between 225 mg and 275 mg of a non-depleting anti-CD4 antibody is administered to a subject in need of treatment. In certain such embodiments, the flat dose of the non-depleting anti-CD4 antibody administered is 250 mg. The non-depleting anti-CD4 antibody is administered alone or in combination with at least one additional compound as described herein, and treatment is sustained until a desired suppression of disease symptoms occurs. The non-depleting anti-CD4 antibody is optionally administered over a period of time in order to maintain in the subject appropriate levels of antibody (or if the antibody is used in combination with a second compound, appropriate levels of the combination of the antibody and second compound) to achieve and maintain suppression of symptoms.

The non-depleting CD4 antibody can be administered by any suitable means, including parenteral, topical, subcutaneous, intraperitoneal, intrapulmonary, intranasal, and/or intralesional administration. Parenteral infusions include intramuscular, intravenous, intraarterial, intraperitoneal, or subcutaneous administration. Intrathecal administration is also contemplated (see, e.g., U.S. Patent Publication No. 2002/0009444 by Grillo-Lopez). In addition, the antibody may suitably be administered by pulse infusion, e.g., with declining doses of the antibody. In certain embodiments, the dosing is given intravenously or subcutaneously. Each exposure may be provided using the same or a different administration means. In one embodiment, each exposure is by subcutaneous administration.

In certain embodiments, a non-depleting anti-CD4 antibody is administered in combination with an insterstitial drug dispersion agent. An interstitial drug dispersion agent is an agent that is capable of degrading or reducing the viscosity of the interstitial matrix. See, e.g., Bookbinder et al., J. of Controlled Release 114:230-241, 2006. The interstitial matrix is a complex three-dimensional dynamic structure that acts as a filter controlling the rate of drug flow. Id. It is comprised of numerous structural macromolecules including, for example, collagens, elastin, and fibronectin, in which glycosaminoglycans and proteoglycans form a hydrated gel-like substance. Id. Glycosaminoglycans such as hyaluronan help create a barrier to bulk fluid flow through the interstitial collagenous matrix by way of their viscosity and water of hydration. Hyaluronan is a mega-dalton molecule containing repeating disaccharide units that allows the extracellular matrix to resist compressive forces. Id. Hydrolysis of glycosaminoglycan, including hyaluronan, reduces the viscosity of the interstitial matrix allowing for an increase in diffusion and absorption of subcutaneously administered fluids and a decrease in infusion site swelling. See, e.g., Pirrello et al., J. of Palliative Medicine 10:861-864, 2007.

Hyaluronidases are a family of glycosaminoglycan-degrading enzymes. One such hyaluronidase is PH20, the predominant hyaluronidase in mammalian testes. PH20 is a neutral pH-active hyaluronidase and degrades glycosaminoglycans under physiologic conditions. rHuPH20 is a soluble form of human hyaluronidase lacking the glycosyl-phosphatidylinositol moiety. (Bookbinder et al., J. of Controlled Release 114:230-241, 2006).

Exemplary insterstitial drug dispersion agents include, but are not limited to, soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Patent Publication Nos. 20050260186 and 20060104968; and in Bookbinder et al., J. of Controlled Release 114:230-241, 2006; Pirrello et al., J. of Palliative Medicine 10:861-864, 2007; and Thomas et al., J. of Palliative Medicine 10: 1312-1320, 2007. In one aspect, a sHASEGP is combined with one or more additional glycosaminoglycanases such as chondroitinases. In one aspect, in the context of non-intravenous parenteral injections (such as intradermal, subcutaneous, intramuscular and other injections into spaces other than the vasculature), a sHASEGP (and/or another glycosaminoglycanase) and another agent (e.g. a co-formulation or a mixture comprising a sHASEGP and a non-depleting anti-CD4 antibody) in a volume of liquid (e.g. a pharmaceutical excipient or other solution) is introduced into a site or sites within the body by injection or infusion.

The methods of the invention include administration of sHASEGP or pharmaceutical compositions containing sHASEGP prior to, simultaneously with, or following administration of a non-depleting anti-CD4 antibody. The sHASEGP polypeptide may be administered at a site different from the site of administration of a non-depleting anti-CD4 antibody or sHASEGP may be administered at a site the same as the site of administration of a non-depleting anti-CD4 antibody. In certain embodiments, sHASEGP is rHuPH20, which is administered at a dose between 0.1 and 15,000 Units, or between 1 and 1000 Units, or between 5 and 500 Units, or between 50 and 300 Units. sHASEGP, including rHuPH20, enzyme activity (Units) can be determined using methods known in the art, for example, a microtiter based hyaluronidase assay as described in Frost et al., Anal. Biochem. 251:263-269, 1997 and U.S. Patent Pub. No. 20060104968.

In certain embodiments, a non-depleting anti-CD4 antibody is administered using, for example, a self-inject device, autoinjector device, or other device designed for self-administration. Various self-inject devices, including autoinjector devices, are known in the art and are commercially available. Exemplary devices include, but are not limited to, prefilled syringes (such as BD HYPAK SCF®, READYFILL™, and STERIFILL SCF™ from Becton Dickinson; CLEARSHOT™ copolymer prefilled syringes from Baxter; and Daikyo Seiko CRYSTAL ZENITH® prefilled syringes available from West Pharmaceutical Services); disposable pen injection devices such as BD Pen from Becton Dickinson; ultra-sharp and microneedle devices (such as INJECT-EASE™ and microinfuser devices from Becton Dickinson; and H-PATCH™ available from Valeritas) as well as needle-free injection devices (such as BIOJECTOR® and IJECT® available from Bioject; and SOF-SERTER® and patch devices available from Medtronic). Co-formulations or co-administrations with such self-inject devices of a non-depleting anti-CD4 antibody with sHASEGP are envisioned, as well as co-formulations or co-administrations of a non-depleting anti-CD4 antibody, sHASEGP and/or at least a second therapeutic compound.

As noted, the non-depleting anti-CD4 antibody can be administered alone or in combination with at least a second therapeutic compound. These second therapeutic compounds are generally used in the same dosages and with administration routes as used heretofore, or about from 1 to 99% of the heretofore-employed dosages. If such second compounds are used, they are used in certain embodiments in lower amounts than if the non-depleting anti-CD4 antibody were not present, so as to eliminate or reduce side effects caused thereby.

Also as noted, a variety of suitable second therapeutic compounds are known in the art, and dosages and administration methods for such second therapeutic compounds have likewise been described. As just one example, the non-depleting anti-CD4 antibody can be administered in combination with cyclophosphamide for treatment of lupus (or MS, rheumatoid arthritis, or inflammatory bowel disease, or other disorder as described herein). A variety of cyclophosphamide treatment regimens have been described in the literature. Exemplary regimens include, but are not limited to, intravenous administration of 0.5-1.0 g/m² monthly for six months than every three months out to 30 months; and intravenous administration of 500 mg every two weeks for three months; oral administration of 1-3 mg/kg per day for twelve weeks or six months. See, e.g., Petri (2004) “Cyclosphosphamide: new approaches for systemic lupus erythematosus” Lupus 13:366-371 and Petri and Brodsky (2006) “High-dose cyclophosphamide and stem cell transplantation for refractory systemic lupus erythematosus” JAMA 295:559-560.

As another example, the non-depleting anti-CD4 antibody can be administered in combination with mycophenolate mofetil (MMF), e.g., CELLCEPT® manufactured by Roche, for the treatment of lupus, including SLE and lupus nephritis. MMF has been used in both induction and maintenance therapy of lupus nephritis. (Appel et al., Nature Clin. Practice 5:132-142 (2009); Ginzler et al., Lupus 14:59-64 (2005)). Various treatment regimens have been described for the use of MMF in the treatment of lupus, including but not limited to, 2.0 g daily for 6 months followed by 1.0 g daily for 6 months; or a range of 0.5 g-2.0 g/day for a period of time ranging from 3-24 months; or a range of 0.5 g-3.0 g daily. Id. In certain instances, MMF is administered in combination with other drugs typically employed for the treatment of lupus such as cyclophosphamide, azathioprine, and/or steroids, such as prednisone. Id.

Administration of the non-depleting anti-CD4 antibody and any second therapeutic compound can be done simultaneously, e.g., as a single composition or as two or more distinct compositions using the same or different administration routes. Alternatively, or additionally, the administration can be done sequentially, in any order. In certain embodiments, intervals ranging from minutes to days, to weeks to months, can be present between the administrations of the two or more compositions. For example, the non-depleting anti-CD4 antibody may be administered first, followed by the second therapeutic compound. However, simultaneous administration or administration of the second therapeutic compound prior to the non-depleting anti-CD4 antibody is also contemplated.

As noted above, a third, fourth, etc. compound is optionally administered in combination with the non-depleting CD4 antibody and the second therapeutic compound. Similarly, treatment for symptoms secondary or related to lupus (e.g., spasticity, incontinence, pain, fatigue) or MS, rheumatoid arthritis, inflammatory bowel disease, or other condition or disease can be administered to the subject, e.g., during treatment with the non-depleting CD4 antibody or combination.

D. Pharmaceutical Formulations

Therapeutic formulations of the antibodies used in accordance with the present invention are prepared for storage by mixing a non-depleting CD4 antibody having the desired degree of purity with optional pharmaceutically acceptable carriers, excipients, or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low-molecular-weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN®, PLURONICS®, or PEG.

Formulations for subcutaneous administration may be, for example, aqueous or lyophilized. Lyophilized formulations adapted for subcutaneous administration are described, for example, in U.S. Pat. No. 6,267,958 (Andya et al.). Such lyophilized formulations may be reconstituted with a suitable diluent to a high protein concentration and the reconstituted formulation may be administered subcutaneously to the mammal to be treated herein. Crystallized forms of the antibody are also contemplated. See, for example, U.S. Patent Publication No. 2002/0136719A1 (Shenoy et al.).

The formulation herein may also contain at least a second compound as necessary for the particular indication being treated, such as those with complementary activities that do not adversely affect each other. For example, it may be desirable to further provide a cytotoxic agent (e.g. methotrexate, cyclophosphamide, or azathioprine), chemotherapeutic agent, immunosuppressive agent, cytokine, cytokine antagonist or antibody, growth factor, hormone, integrin, integrin antagonist or antibody (e.g., an LFA-1 antibody, or an alpha 4 integrin antibody such as natalizumab), interferon class drug such as IFN-beta-1a or IFN-beta-1b, an oligopeptide such as glatiramer acetate, intravenous immunoglobulin (gamma globulin), lymphocyte-depleting drug (e.g., mitoxantrone, cyclophosphamide, CAMPATH® antibodies, or cladribine), non-lymphocyte-depleting immunosuppressive drug (e.g., MMF or cyclosporine), cholesterol-lowering drug of the “statin” class, estradiol, drug that treats symptoms secondary or related to lupus, MS, rheumatoid arthritis, or inflammatory bowel disease (e.g., spasticity, incontinence, pain, fatigue), a TNF inhibitor, DMARD, NSAID, corticosteroid (e.g., methylprednisolone, prednisone, dexamethasone, or glucocorticoid), levothyroxine, cyclosporin A, somatostatin analogue, anti-metabolite, a T- or B-cell surface antagonist/antibody, etc., or others as noted above in the formulation. The type and effective amounts of such other agents depend, for example, on the amount of antibody present in the formulation, the type of lupus, MS, rheumatoid arthritis or other condition or disease being treated, and clinical parameters of the subjects.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug-delivery systems (for example, liposomes, albumin microspheres, microemulsions, nano-particles and nanocapsules) or in macroemulsions. Such techniques are disclosed, e.g., in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the non-depleting antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid.

The formulations to be used for in vivo administration must be sterile. This is readily accomplished, for example, by filtration through sterile filtration membranes.

E. Articles of Manufacture

In another embodiment of the invention, an article of manufacture containing materials useful for the treatment of lupus, MS, rheumatoid arthritis, inflammatory bowel disease, or other condition or disease described above is provided. The article of manufacture comprises (a) a container comprising a composition comprising a non-depleting CD4 antibody and a pharmaceutically acceptable carrier or diluent within the container; and (b) a package insert with instructions for treating lupus, MS, rheumatoid arthritis, inflammatory bowel disease, or other condition or disease in a subject by administration of the antibody, alone or in combination with at least a second compound.

The package insert is on or associated with the container. Suitable containers include, for example, bottles, vials, syringes, etc. The containers may be formed from a variety of materials such as glass or plastic. The container holds or contains a composition that is effective for treating the lupus, MS, rheumatoid arthritis, inflammatory bowel disease, or other condition or disease and may have a sterile access port (for example, the container may be an intravenous solution bag or a vial having a stopper pierceable by a hypodermic injection needle). At least one active agent in the composition is the non-depleting CD4 antibody. The label or package insert indicates that the composition is used for treating lupus, MS, rheumatoid arthritis, inflammatory bowel disease, or other condition or disease in a subject eligible for treatment with specific guidance regarding dosing amounts and intervals of antibody and any other drug being provided.

The article of manufacture may further comprise a second container comprising a pharmaceutically acceptable diluent buffer, such as bacteriostatic water for injection (BWFI), phosphate-buffered saline, Ringer's solution, and dextrose solution. The article of manufacture optionally comprises a second or third container comprising a second compound, such as any of those described herein, where the article further comprises instructions on the package insert for treating the subject with the second compound. Alternatively, the composition comprising the non-depleting CD4 antibody can also comprise the second compound. The article of manufacture may further include other materials desirable from a commercial and user standpoint, including other buffers, diluents, filters, needles, and syringes.

### III. EXAMPLES

Further details of the invention are illustrated by the following non-limiting Examples. The disclosures of all citations in the specification are expressly incorporated herein by reference.

### Example 1

### Non-Depleting Anti-CD4 Variants with Minimized Effector Functions and Decreased Clearance In Vivo

The concern with targeting T cells with anti-CD4 antibodies has been reduction or depletion that could lead to immune suppression. In addition, clinical results with prior anti-CD4 antibodies, as discussed above in the background section, indicate that more desirable dosing regimens of anti-CD4 antibodies are needed. Accordingly, anti-CD4 antibody variants (see Table 2 below) were engineered to be non-depleting via certain amino acid substitutions in the parent molecule. Specifically, asparagine at amino acid position 297 in the heavy chain was changed to alanine (N297A). This substitution has been shown to abrogate the N-linked glycosylation at the Fc region which has been shown to be important for binding of antibody to Fcγ receptors (Burton and Dwek, Science 313:627-28, 2006). In addition, it has been shown that aglycosylated antibodies fail to induce ADCC both in vitro and in vivo (Isaacs et al., J. Immunol. 148:3062-71, 1992; Lund et al., Mol. Immunol. 29:53-9, 1992). Lack of FcγR interaction may provide improved safety because of the lack of T-cell depletion and potential for reduced infusion reactions, both of which are mediated through the Fcγ receptors.

In addition, because of the rapid clearance resulting from CD4-mediated elimination, frequent dosing may be required to maintain CD4 downmodulation and saturation. It has been shown previously that antibodies engineered with increased FcRn binding demonstrate an increased half-life and more prolonged exposure in monkeys (Hinton et al., J. Immunol. 176:346-56, 2006). This may be the first study, however, testing the affect of an antibody, in particular an anti-CD4 antibody, engineered to both lack glycosylation and to affect binding to FcRn on half-life in vivo. Accordingly, non-depleting anti-CD4 antibody variants (see Table 2 below) were also engineered to affect binding to FcRn. Specifically, asparagine at amino acid position 434 in the heavy chain was changed to alanine (N434A) or histidine (N434H). Certain non-depleting anti-CD4 variants were tested for binding to CD4+ T cells from humans and non-human primates, for binding to Fcγ receptors and FcRn, and were also assessed for effector functions such as antibody-dependent cell-mediated cytotoxicity (ADCC) and complement-dependent cytotoxicity (CDC), as described in the following experiments. In addition, certain non-depleting anti-CD4 variants were tested in vivo and clearance of the variants monitored along with CD4 T cell receptor occupation, as described below.

TABLE 2
Anti-CD4 antibody variants.

| Antibody | | LC Variant AA | | | HC Variant AA | |
|---|---|---|---|---|---|---|
| Variant | Light Chain | position | AA | Heavy Chain | position | AA |

| | | | | | | |
|---|---|---|---|---|---|---|
| A | LC1 | 117 | P | HC1 | 297 | N |
| | (SEQ ID NO.: 1) | | | (SEQ ID NO.: 3) | 434 | N |
| B | LC1 | 117 | P | HC2 | 297 | A |
| | (SEQ ID NO.: 1) | | | (SEQ ID NO.: 4) | 434 | N |
| C | LC1 | 117 | P | HC3 | 297 | A |
| | (SEQ ID NO.: 1) | | | (SEQ ID NO.: 5) | 434 | A |
| D | LC1 | 117 | P | HC4 | 297 | A |
| | (SEQ ID NO.: 1) | | | (SEQ ID NO.: 6) | 434 | H |
| E | LC2 | 117 | L | HC1 | 297 | N |
| | (SEQ ID NO.: 2) | | | (SEQ ID NO.: 3) | 434 | N |
| F | LC2 | 117 | L | HC2 | 297 | A |
| | (SEQ ID NO.: 2) | | | (SEQ ID NO.: 4) | 434 | N |
| G | LC2 | 117 | L | HC3 | 297 | A |
| | (SEQ ID NO.: 2) | | | (SEQ ID NO.: 5) | 434 | A |
| H | LC2 | 117 | L | HC4 | 297 | A |
| | (SEQ ID NO.: 2) | | | (SEQ ID NO.: 6) | 434 | H |

### Measurement of CD4 Binding Affinity by Equilibrium Binding Analysis

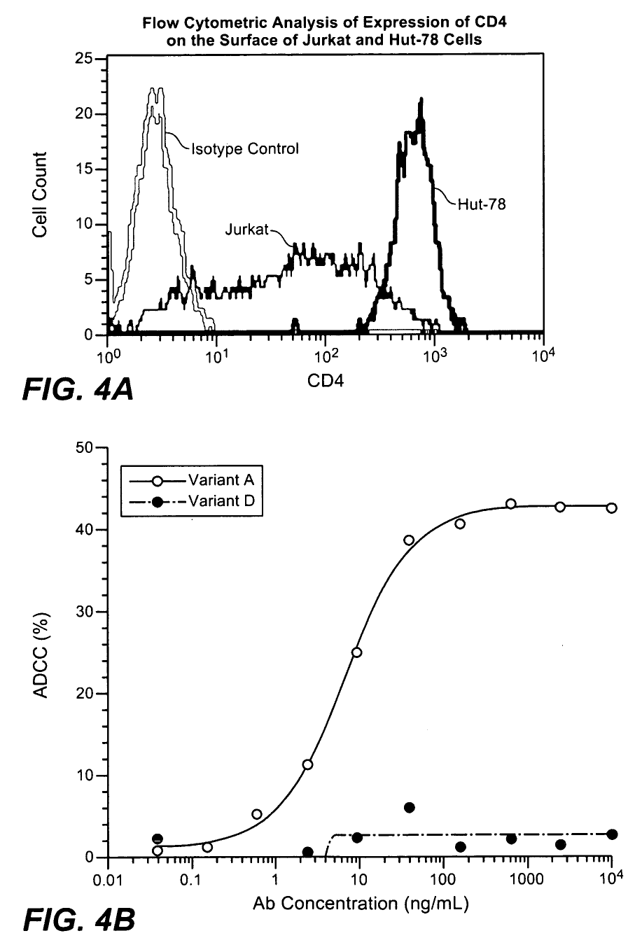

The Jurkat human T-cell leukemic line expresses CD4 (see FIG. 4A) and was utilized to determine the affinity of Variant B and Variant D for CD4 by equilibrium binding analysis. Variant B is similar to Variant D except that Variant B carries the normal (wild-type) amino acid at position 434, N434 (see Table 2). Equilibrium binding measurements were carried out as follows.

CD4+ Jurkat cells were cultured in growth media, which contained RPMI 1640 media supplemented with 10% fetal bovine serum (FBS), 2 mM L-glutamine, 1× penicillin-streptomycin, at 37° C. in 5% CO₂. Cells were washed with binding buffer (50:50 DMEM/F12 with 2% FBS and 50 mM Hepes, pH 7.2) and were placed into 96-well plates at approximately 2.3×10⁵ cells/well in 0.2 mL of binding buffer. The non-depleting anti-CD4 antibody variants (Variant B and Variant D) were iodinated using the Iodogen method. The radiolabeled antibodies were purified from free 125I-NA by gel filtration using a NAP-5 column; the purified Variant D antibody had a specific activity of 16.44 μCi/μg and the Variant B antibody, a specific activity of 13.37 μCi/μg. Competition mixtures containing a fixed concentration of iodinated antibody and decreasing concentrations of serially diluted unlabeled antibody were added to the cells and then incubated for 4 hours at 4° C. The final concentration of the iodinated antibody in each incubation with cells was approximately 25 pM (2.5×10⁴ cpm/0.25 mL) and the final concentration of the unlabeled antibody in the incubations with cells varied, starting at 50 nM and decreasing by 2-fold for 11 serial dilutions. Competition reactions were run in triplicate. The cells were transferred to a Millipore Multiscreen filter plate and washed four times with binding buffer to separate the free from bound iodinated antibody. The filters were counted on a Wallac Wizard 1470 gamma counter (Perkin Elmer Life and Analytical Sciences Inc.; Wellesley, Mass.). The binding data were evaluated using NewLigand software (Genentech), which uses the non-linear regression fitting algorithm of Munson and Rodbard, Anal. Biochem. 107:220-39, 1980, to determine the binding affinity of the antibody.

Saturation binding curves and Scatchard plots derived from linearized transformation of the data were generated and analyzed (data not shown). The designated affinities, or K_{D}s for binding of Variant B and Variant D to Jurkat cells were derived from a non-linear regression curve-fitting of the raw binding data (Munson and Rodbard, Anal. Biochem. 107:220-39, 1980). The data showed comparable equilibrium binding constants for Variant D and Variant B of 65 pM and 62 pM, respectively, and demonstrate that the substitution at amino acid 434 in Variant D (N434H) did not alter antibody binding to CD4.

### Binding of Variant D to Human, Baboon, Cynomolgus Monkey, and Rhesus Monkey CD4+ T Cells as Measured by Flow Cytometry

To characterize binding of Variant D to primary human CD4+ T cells and to determine its cross-reactivity to CD4 from non-human primate species, saturation binding titration experiments were performed on cells from humans, baboons, cynomolgus monkeys, and rhesus monkeys. Binding was analyzed and quantified by flow cytometry as described below.

Fresh human blood from a healthy human donor was obtained. Non-human primate blood was obtained from Southwest Foundation for Biomedical Research in San Antonio, Tex. (i.e., baboon, cynomolgus monkey, and rhesus monkey). Each sample of blood was diluted with an equivalent volume of PBS, overlaid on Ficoll (GE Healthcare; Princeton, N.J.), then centrifuged to isolate mononuclear cells. Residual red blood cells were lysed using erythrocyte lysis buffer (Qiagen; Valencia, Calif.) and washed. Cells were bound with serial titrations of either Variant D antibody or a control monoclonal antibody containing human IgG1 Fc similar to that of Variant D but lacking the modifications of Variant D and incubated on ice for 30 minutes and washed. Cells were then incubated with Fcγ-specific human IgG PE-conjugated antibody (Jackson ImmunoResearch Laboratories, Inc.; West Grove, Pa.) at 20 μg/mL for another 30 minutes on ice to detect the quantity of anti-CD4 antibody bound. Cells were washed, then co-stained with anti-CD3 fluorescein isothiocyanate (FITC) and anti-CD8 allophycocyanin (APC) (BD Biosciences) at saturating concentrations for 30 minutes on ice and then washed. These antibodies provided a means to gate on CD4+ T cells independent of Variant D binding. Samples were run on a FACSCalibur flow cytometer (BD Biosciences) and analyzed using Flowjo software. The mean fluorescence intensity (MFI) of CD4 staining was plotted as a function of the concentration of Variant D present during binding. Half-maximal effective concentration (EC50) values were calculated from the binding curves using a four-parameter curve-fitting algorithm (KaleidaGraph™ software).

The binding profiles of Variant D on human and baboon CD4+ T cells were relatively similar with CD4 saturation occurring at comparable antibody concentrations (data not shown). In contrast, concentrations three orders of magnitude higher were required to saturate CD4 on T cells from rhesus and cynomolgus monkeys than those required for the human and the baboon (data not shown). The control antibody showed no detectable binding to CD4+ T cells from any of the species.

For most antibody/antigen interactions, binding affinities can be approximated from the antibody concentration required to achieve half-maximal binding, i.e., the EC50 concentration (Wessels et al., Proc. Natl. Acad. Sci. USA 84:9170-74, 1987; Neri et al., Trends in Biotechn. 14:465-70, 1996). The estimated affinities (EC50 values) of Variant D for human and baboon CD4+ T cells were 0.17 and 0.14 nM, respectively, whereas the values for rhesus and cynomolgus monkey CD4 were substantially higher (180 and 177 nM, respectively) as shown in Table 3 below. The flow cytometry-based EC50 values presented in Table 3 present the mean values from four experiments in human and baboon cells and from two experiments in rhesus and cynomolgus monkeys.

TABLE 3
EC50 Values (nM) of Variant D for CD4 on T cells Based on Flow
Cytometry Analysis

| Species | Variant D |
|---|---|
| Human | 0.17 ± 0.08 |
| Baboon | 0.14 ± 0.05 |
| Cynomolgus monkey | 180.6 |
| Rhesus monkey | 177.7 |

^{a}EC50 = half-maximal effective concentration

### Fcγ Receptor Binding

The binding affinities of various antibodies to FcγRIA, FcγRIIA, FcγRIIB, and two allotypes of FcγRIIIA (F158 and V158) were measured in ELISA-based ligand-binding assays using the respective recombinant Fcγ receptors. Purified human Fcγ receptors were expressed as fusion proteins containing the extracellular domain of the receptor γ chain linked to a Gly/6×His/glutathione S-transferase (GST) polypeptide tag at the C-terminus. The binding affinities of antibodies to those human Fcγ receptors were assayed as follows.

For the low-affinity receptors, i.e. FcγRIIA (CD32A), FcγRIIB (CD32B), and the two allotypes of FcγRIIIA (CD16), F-158 and V-158, antibodies were tested as multimers by cross-linking with a F(ab′)2 fragment of goat anti-human kappa chain (ICN Biomedical; Irvine, Calif.) at an approximate molar ratio of 1:3 antibody:cross-linking F(ab′)₂. Plates were coated with an anti-GST antibody (Genentech) and blocked with bovine serum albumin (BSA). After washing with phosphate-buffered saline (PBS) containing 0.05% Tween-20 with an ELx405™ plate washer (Biotek Instruments; Winooski, Vt.), Fcγ receptors were added to the plate at 25 ng/well and incubated at room temperature for 1 hour. After the plates were washed, serial dilutions of test antibodies were added as multimeric complexes and the plates were incubated at room temperature for 2 hours.

Following plate washing to remove unbound antibodies, the antibodies bound to the Fcγ receptor were detected with horseradish peroxidase (HRP)-conjugated F(ab′)₂ fragment of goat anti-human F(ab′)₂ (Jackson ImmunoResearch Laboratories; West Grove, Pa.) followed by the addition of substrate, tetramethylbenzidine (TMB) (Kirkegaard & Perry Laboratories; Gaithersburg, Md.). The plates were incubated at room temperature for 5-20 minutes, depending on the Fcγ receptors tested, to allow color development. The reaction was terminated with 1 M H₃PO₄ and absorbance at 450 nm was measured with a microplate reader (SpectraMax® 190, Molecular Devices; Sunnyvale, Calif.). Dose-response binding curves were generated by plotting the mean absorbance values from the duplicates of antibody dilutions against the concentrations of the antibody. Values for the effective concentration of the antibody at which 50% of the maximum response from binding to the Fcγ receptor was detected (EC₅₀) were determined after fitting the binding curve with a four-parameter equation using SoftMax Pro (Molecular Devices).

For the high-affinity receptor (FcγRIA), the antibodies were assayed as monomers without cross-linking. The rest of the assay procedures were the same as those for low-affinity receptors.

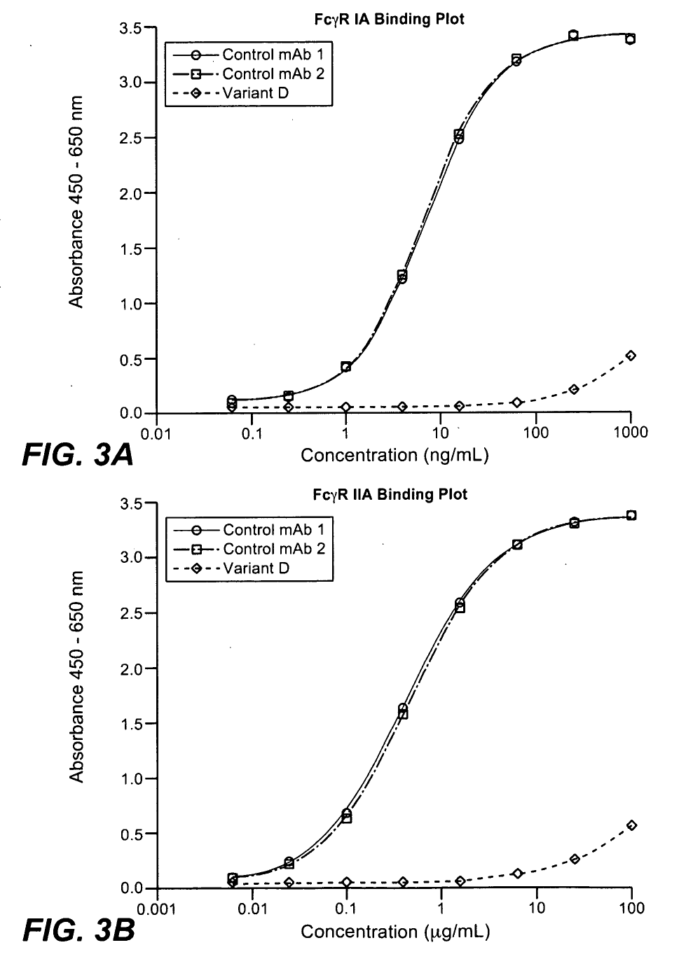

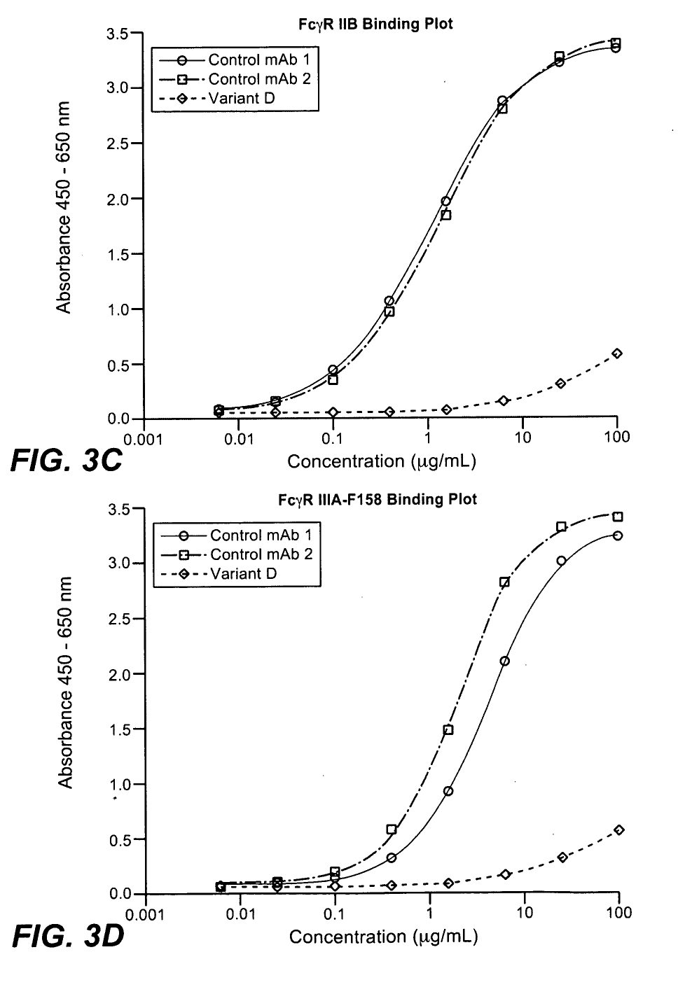

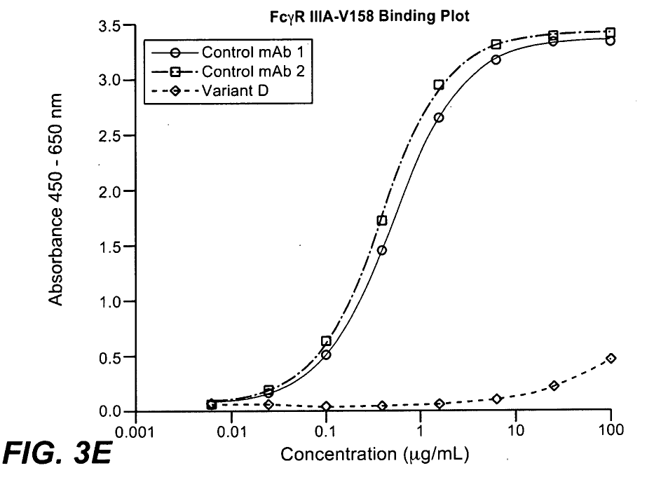

The binding affinities of non-depleting anti-CD4 antibody variant D (see Table 2) (Variant D) to FcγRIA, FcγRIIA, FcγRIIB, and two allotypes of FcγRIIIA (F158 and V158) were measured in the ELISA-based ligand binding assays as described above. Two different control monoclonal antibodies, each of which contain human IgG1 Fc similar to that of Variant D but lacking the modifications of Variant D, were tested as positive controls. Samples were tested in duplicate and a total of three independent experiments were performed for each Fcγ receptor. Binding curves from representative experiments are presented in FIGS. 3A-E.

The results shown in FIGS. 3A-E were obtained from one representative run of an in vitro binding experiment with Variant D and two control monoclonal antibodies. The antibodies were assayed in monomeric form for binding with FcγR1 and multimeric form for binding with the other Fcγ receptors tested. All data points were collected in duplicate and the mean of the duplicate absorbance values was plotted against the antibody concentration. As shown in FIGS. 3A-E, the two control monoclonal antibodies appeared to bind similarly to each of the Fcγ receptors tested, whereas Variant D showed dramatically reduced binding to all the Fcγ receptors tested. The minimum binding toward Fcγ receptors by Variant D was consistent with published results referencing human IgG1 antibodies carrying mutation at the amino acid 297 position (Lund et al., Mol. Immunol. 29:53-9, 1992; Shields et al., J. Biol. Chem. 276:6591-604, 2001). This mutation abrogates the N-linked glycosylation at the Fc region which has been shown to be important for binding of antibody to Fcγ receptors (Burton and Dwek, Science 313:627-28, 2006). It is reasonable to conclude that Variant D binds to FcγRs at affinities significantly lower than those of wild-type human IgG1 antibodies as a result of the engineered amino acid substitution at amino acid position 297.

ADCC Assay with Peripheral Blood Mononuclear Cells

ADCC assays were carried out using peripheral blood mononuclear cells (PBMCs) from healthy donors as effector cells, and two human T-lymphoma cell lines, Jurkat and Hut-78 (American Type Culture Collection [ATCC], Manassas, Va.), as target cells. To minimize donor variations derived from allotypic differences at residue 158 position of FcγRIIIA, blood donors were limited to those carrying the heterozygous FcγRIIIA allotype (F/V158). Briefly, PBMCs were isolated from fresh blood of healthy human donors by Ficoll-Paque™ (GE Healthcare, Sweden) density gradient centrifugation. Target cells (4×10⁴) prepared in assay medium (RPMI-1640 with 1% BSA and 100 units/mL penicillin/streptomycin) were seeded in each well of 96-well, round-bottom tissue culture plates. Serial dilutions of antibodies were added to the plates containing the target cells at 50 μL/well followed by incubation at 37° C. with 5% CO₂ for 30 minutes to allow opsonization. The final concentrations of antibodies ranged from 10000 to 0.0038 ng/mL following serial four-fold dilutions. After the incubation, PBMC effector cells (1.0×10⁶) in assay medium were added to each well to give a ratio of 25:1 effector:target cells and the plates were incubated for an additional 4 hours. The plates were centrifuged at the end of incubation and the supernatants were assayed for lactate dehydrogenase (LDH) activity using a Cytotoxicity Detection Kit (Roche Diagnostics Corporation; Indianapolis, Ind.). Cell lysis was quantified through absorbance at 490 nm using a microplate reader (SpectraMax® 190, Molecular Devices; Sunnyvale, Calif.). Absorbance of wells containing only the target cells served as the control for background (Low Control), whereas wells containing target cells lysed with Triton-X100 provided maximum signal available (High Control). Antibody-independent cellular cytotoxicity (AICC) was measured in wells containing target and effector cells without the addition of antibody. The extent of specific ADCC was calculated as follows:

$$\% ADCC = 100 \times \frac{A 490 nm ( Sample ) - A 490 nm ( AICC )}{\begin{matrix}A 490 nm ( High Control ) - \\ A 490 nm ( Low Control )\end{matrix}}$$

The mean ADCC values from duplicates of antibody sample dilutions were plotted against the antibody concentration, and the EC₅₀ values were generated by fitting the data to a four-parameter equation using SoftMax Pro.

For flow cytometric analysis, a fluorescein-conjugated mouse monoclonal antibody against human CD4 (clone RPA-T4) and a fluorescein-conjugated isotype control, mouse monoclonal antibody MOPC-1, were obtained from BD Biosciences (San Jose, Calif.). Cells were stained with antibodies as recommended by the manufacturer. Five thousand live-gated events were acquired from each sample using a FACSCalibur™ flow cytometer (BD Biosciences). Data were analyzed using the CellQuest™ software program (BD Biosciences).

ADCC is a well-recognized immune effector function in which antigen-specific antibodies direct effector cells of the innate immune system to kill antigen-expressing target cells. In this study, purified PBMCs from the blood of healthy donors were used to assess the potential of Variant D for ADCC activity. Two human T-lymphoma cell lines, Jurkat and Hut-78, were used as target cells as described above. The expression of CD4 on surface of both target cell types was verified by flow cytometry analysis (see FIG. 4A). Variant A is similar to Variant D except that Variant A contains the normal (wild-type) human IgG1 Fc region without amino acid substitutions (i.e. at positions 297 and 434), and was tested as a positive control. The antibodies were assayed at least twice with each target cell line using PBMCs from different donors. Percent ADCC was plotted against concentration of the antibodies and the data were fitted with a four-parameter model. The ADCC curves from one representative experiment in which Variant A and Variant D were assayed with Hut-78 cells are presented in FIG. 4B. While ADCC was observed with the control antibody, Variant A, no ADCC activity was observed with Variant D at concentrations up to 10 μg/mL. Similar results were obtained in experiments using Jurkat cells as target cells (data not shown). The lack of ADCC by Variant D in the present study is consistent with published results that aglycosylated antibodies failed to induce ADCC both in vitro and in vivo (Isaacs et al., J. Immunol. 148:3062-71, 1992; Lund et al., Mol. Immunol. 29:53-9, 1992).

### Complement-Dependent Cytotoxicity Assay

The complement-dependent cytotoxicity (CDC) assays were carried out using complement derived from human serum (Quidel Corporation; San Diego, Calif.) with Hut-78 or Jurkat cells as target cells. The antibody samples were serially diluted in assay medium (RPMI-1640 medium supplemented with 20 mM Hepes pH 7.2, 0.1% BSA, and 0.1 mg/mL gentamicin), and distributed into a 96-well tissue culture plate (Costar® Corning Inc.; Acton, Mass.). Following the addition of human serum complement (diluted 1:3 in assay medium) and the target cells (10⁵ cells/well), the plate was incubated with 5% CO₂ for 1-2 hours at 37° C. After the incubation, AlamarBlue™ was added at 50 μL/well and the plate was incubated for an additional 15-18 hours. The CDC activity of the test antibody was quantified through absorbance at 530 nm excitation with 590 nm emission on a fluorescence plate reader (SpectraMax GeminiXS, Molecular Devices). The EC₅₀ values were generated by fitting the data to a four-parameter equation (SoftMax Pro).

Complement-dependent cytotoxicity (CDC) is a cell-killing mechanism in which complement-dependent cell lysis occurs as a result of binding of antibody to C1q, which leads to activation of the complement pathway. In this study, the ability of Variant D to induce CDC activity was assessed using normal human serum complement and Hut-78 and Jurkat human T-lymphoma cell lines, which express CD4 on the cell surface. Variant A, which contains the normal (wild-type) human IgG1 Fc region without amino acid substitutions (i.e. at positions 297 and/or 434), was a positive control. The viability of cells in the presence of the antibodies and human serum complement were measured with AlamarBlue™. AlamarBlue™ is a non-toxic indicator dye that yields a calorimetric change and a fluorescent signal in response to metabolic activities of living cells. The assay signal is proportional to the number of viable cells; hence, the degree of reduction of signal indicates the extent of cytotoxicity induced by the antibodies. In this study, three independent CDC assay runs were performed, two with Hut-78 and one with Jurkat as target cells. There was no detectable CDC activity observed for either Variant A or Variant D in all three experiments. The lack of CDC activity with Variant D was consistent with published reports that depletion of carbohydrate in human IgG1 abrogated its binding to C1q and prevented the activation of the complement system (Tao and Morrrison, J. Immunol. 143:2595-601, 1989). The lack of CDC activity with Variant A, which contains the normal IgG1 Fc region and showed active ADCC in the assay (see above), is somewhat difficult to explain. It has been shown that tumor cell lines could display differential sensitivity toward ADCC and CDC and that susceptibility to CDC could be affected by a number of factors including receptor density and expression level of complement regulatory proteins (Gelderman et al., Trends Immunol. 25:158-64, 2004; van Meerten et al., Clin. Cancer Res. 12:4027-35, 2006). It is possible that both Hut-78 and Jurkat cells are resistant to CDC by the Variant A and Variant D antibodies at the concentrations tested in the present assay system. Nevertheless, the absence of a true positive control in the study prevented an absolute conclusion regarding potential CDC activity of Variant D.

The results described above indicate that Variant D binds to Fcγ receptors with minimum affinities (based on binding curves) and was unable to induce ADCC or CDC in vitro. The lack of effector functions of Variant D is consistent with published results for aglycosylated antibodies.

### FcRn Binding

The following experiments were carried out to assess the relative affinity of certain non-depleting anti-CD4 variants to human and baboon FcRn. The variants tested included Variant B and Variant D. Variant B is similar to Variant D except that Variant B carries the normal (wild-type) amino acid at position 434, N434 (see Table 2).

The IC₅₀s of Variant B and Variant D binding to human and baboon FcRn were measured using the BIAcore 3000 surface plasmon resonance system (BIAcore Inc, Piscataway, N.J.; Lofas and Johnsson 1990; Karlsson et al. 1991). The amine-coupling method used for immobilization of a control monoclonal antibody which contains human IgG1 Fc similar to that of Variant D but lacking the modifications of Variant D (Control Antibody) onto a carboxymethylated dextran biosensor chip (Sensor Chip CM5, BIAcore) was essentially as described in the manufacturer's instructions (BIAcore 1991; Johnsson et al. 1991). Briefly, the biosensor chip was activated using N-ethyl-N′-(3-dimethylaminopropyl) carbodiimide hydrochloride (EDC HCl) mixed with N-hydroxysuccinimide (NHS). Control Antibody, diluted to 12 μg/ml in 10 mM sodium acetate, pH 4, was injected over the chip to give an immobilized antibody signal of approximately 3000 RUs. Unreacted succinimide groups were then blocked with an injection of 1 M ethanolamine, giving a final density of approximately 2000 RUs of Control Antibody.

Soluble histidine-tagged human FcRn (SEQ ID NO: 13) and soluble histidine-tagged baboon FcRn (SEQ ID NO: 14) were produced from transient expression in CHO cells using standard methods well known to one skilled in the art. Each of the histidine-tagged FcRn polypeptides was purified using nickel column chromatography according to methods well known to one skilled in the art. The histidine tags were not removed from the FcRn polypeptides following purification. For simplicity, the histidine-tagged FcRn polypeptides are subsequently referred to as “FcRn” in the discussion of assay methodology and results below but it is understood that the histidine tags were still attached to the polypeptides. Each of Variant B and Variant D were serially diluted in running buffer (PBS containing 0.05% Tween-20 pH 6) and incubated at room temperature with a constant (100 nM) concentration of FcRn for 30 min before injection.

The final concentrations of antibodies used ranged from 2.29 nM to 5 μM. An FcRn calibration curve was prepared concurrently, using serial dilutions of known concentrations of FcRn. The FcRn and antibody mixtures were injected over the chip, and report points taken at 50 seconds after the start of injection. Buffer blanks, antibody binding in the absence of FcRn, and a reference flow cell were used as negative controls to adjust the values of the report points. The results were then converted to concentrations of free FcRn using the FcRn calibration curve.

Concentrations of free FcRn were plotted against the log of antibody concentrations in GraphPad Prism. The IC₅₀ of binding was determined by fitting the data to a 4 parameter curve (Y=m4+(m3−m4)/(1+10̂((log IC50−X)*Hillslope)) where m3 is the maximal signal, m4 is the minimal signal and the Hillslope is a slope constant. Experiments were repeated at least three times, and the IC₅₀s were reported with standard error of the mean (SEM).

The results showed that Variant D binds to both human and baboon FcRn with similar relative affinity and approximately 3.5-fold more tightly than does Variant B at pH 6. IC₅₀ values for Variant D binding to human and baboon FcRn were 144.1 nM and 160.2 nM respectively, while Variant B exhibited significantly higher values of 496.0 nM and 557.9 nM respectively (Table 4).

TABLE 4
Binding to human and baboon FcRn^{a} at pH 6.0 as measured
by BIAcore analysis

| | Human FcRn | Baboon |
|---|---|---|
| | IC50 (nM) | FcRn IC50 (nM) |
| Variant B | 496.0 ± 38.5 | 557.9 ± 36.2 |
| Variant D | 144.1 ± 16.3 | 160.2 ± 23.1 |

^{a}values represent the average from three expts.

In vivo clearance of non-depleting anti-CD4 variants

The following experiments were carried out to assess in vivo clearance of certain non-depleting anti-CD4 variants in baboons. The variants tested included Variant B, Variant C, and Variant D. Variant B is similar to Variant D except that Variant B carries the normal (wild-type) amino acid at position 434, N434 (see Table 2). Variant C is similar to Variant D except that Variant C carries a different amino acid substitution at position 434, N434A (see Table 2).

### Species

Twenty male and 20 female purpose-bred, drug-naive olive baboons (African origin; Southwest Foundation for Biomedical Research; San Antonio, Tex.) were acclimated for at least 7 days before dosing. The animals were 2-4 years old and weighed 5-12 kg at prestudy screening. Only animals that appeared to be healthy and that were free of obvious abnormalities were used for the study.

### Study Design

The animals were randomized into four groups; each animal received four doses of Vehicle or test material by slow IV infusion injection. Animals in Group 1 (2 males and 2 females) received Vehicle at 4.0 mL/kg. Animals in Groups 2, 3, and 4 (6 males and 6 females per group) received 40 mg/kg of Variant B, Variant C, or Variant D, respectively, administered in doses of 3.9 to 4.1 mL/kg doses. Doses were given twice weekly over a 2-week period (on Days 1, 4, 8, and 12).

### Dose Preparation

Test articles at their final concentrations were made up in single-use vials. Before administration, frozen test article vials (Groups 2-4) were thawed overnight in a refrigerator set to maintain a temperature range of 2° C.-8° C. On each day of dose administration, thawed test article and vehicle vials were equilibrated at ambient temperature for approximately 30 minutes before dose solutions were prepared. Vials were gently swirled, then the contents of individual vials were combined into a single depyrogenated sterile glass container specific to each dosing group. Dose solutions were used within 6 hours of preparation.

### Dose Administration

On Days 1, 4, 8, and 12, animals received a continuous IV infusion (3-4 mL/min) of test article via a superficial vein on the arm or leg, followed by a 1-mL saline flush.

### Body Weight Measurements

Individual animals body weights were collected on Day 7 of the acclimation period, Day 7 of dose administration, and recovery Days 7, 21, 35, 45, and 56/57.

### Blood Sample Collection

Blood samples were collected from each animal by venipuncture into a peripheral vein.

### Sample Collection

Blood samples for PK analysis (1 mL) were collected and transferred into serum-separator tubes at the following timepoints:

≦1 hour predose and 1 hour post-dose on Days 1, 4, and 8

≦1 hour predose, and 1, 6, and 12 hours post-dose, on Day 12

On Days 2, 5, 9, 13, 14, 16, 19, 22, 24, 26, 28, 30, 33, 36, 40, 43, 47, 54, 57, 61, and 64

Before necropsy on Day 68 or 69

For PK analysis, the timepoints began at Day 0 (Study Day 1=PK Day 0) and are recorded as Days 0, 0.042, 1, 2.958, 3.042, 4, 6.958, 7.042, 8, 10.958, 11.042, 11.25, 11.5, 12, 13, 15, 18, 21, 23, 25, 27, 29, 32, 35, 39, 42, 46, 53, 56, 60, 63, and 67 (Study Day 68).

### Blood Sample Processing

### Sample Processing

Samples for PK analysis were allowed to clot at room temperature for 20-60 minutes. Clotted samples were centrifuged within 1 hour of collection at a relative centrifugal force of 1,500-2,000×g for 10-15 minutes at 2° C.-8° C. Approximately 500 μL of serum was separated from the samples within approximately 20 minutes of the end of centrifugation and transferred into prelabeled EPPENDORF® tubes. The tubes were kept on dry ice or stored immediately in a freezer set to maintain a temperature of −60° C. to −86° C. until the samples were assayed.

### Assays

### Serum Anti-CD4 Antibody Concentration Assay (ELISA)

The concentration of anti-CD4 antibodies in serum was determined using an ELISA assay. Human soluble CD4 (rCD4, Genentech) diluted to 1 μg/mL in phosphate-buffered saline (PBS) was coated onto polystyrene 384-well MaxiSorp™ plates (No. 464718, Nalgate NUNC®, Sigma-Aldrich; St. Louis, Mo.). After 16-120 hours, the coating was removed and plates were blocked with block buffer (PBS/0.5% bovine serum albumin (BSA)/Proclin) for 0.5-3 hours. Dilutions of anti-CD4 Variant B standards (0.39-50 ng/mL) were prepared in assay buffer (PBS/0.5% BSA/0.05% Tween 20/Proclin) from a 10 μg/mL standard stock. Assay performance was monitored using three levels of controls. Controls were made by spiking baboon serum with three concentrations of Variant B. Controls were diluted 1:20 in assay buffer on each assay day. Samples were diluted to a minimum dilution of 1:20 in assay buffer, then diluted further into assay range. Blocked plates were washed three times with wash buffer (PBS/0.05% Tween 20), and standards, controls, and samples were added to appropriate assay wells. After a 1.5-hour incubation, plates were washed six times. Bound Variant B was detected by adding monkey IgG-absorbed, horseradish peroxidase-conjugated rabbit anti-human IgG antibody (No. CUS1684.H, Binding Site; San Diego, Calif.) diluted in assay buffer. After a 1-hour incubation, plates were washed six times and substrate (TMB Substrate No. 50-65-02, KPL; Gaithersburg, Md.) was added to all assay wells. The substrate reaction was stopped after 20 minutes with 1 M phosphoric acid. Plates were read at 450 nM using a reference wavelength of 620 nM. Sample concentrations were determined by comparing results with standards using a four-parameter curve-fit algorithm. The assay limit of quantitation (LOQ) is 0.016 μg/1 mL.

### Methods for Determination of PK Parameters

### Noncompartmental PK Analysis

Animals were dosed by IV infusion (3-4 mL/min) for 8.25 to 12.26 minutes. For analytical purposes, serum concentration-time data from each animal were analyzed using the IV bolus model of input (Model 201, WinNonlin-Pro, version 5.0.1, Pharsight Corporation; Mountain View, Calif.). The following methods were used to estimate specific PK parameters:

AUCLₐₗₗ=area under the serum concentration-time curve to the last observation point

CL=clearance (Dose/AUC_{inf})

Vss=volume of distribution at steady state (MRT_{inf}•CL)

Non-Depleting Anti-CD4 Antibody Concentrations in Serum

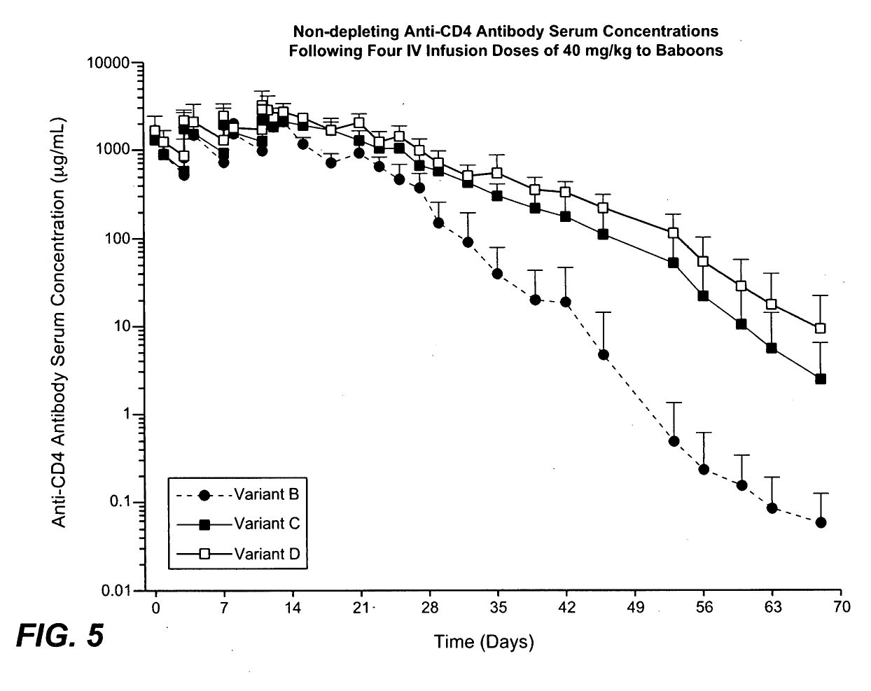

The mean (±SD) serum concentration profiles are presented in FIG. 5. In addition, CD4 receptor saturation and modulation on peripheral-blood T cells was measured by flow cytometry. One hour following the first dose of Variant D, >98% of CD4 receptors on peripheral T lymphocytes were saturated, compared with the predose timepoint (data not shown). In addition, a 30%-40% downmodulation of CD4 receptors on peripheral T lymphocytes was observed starting 3 days after administration of the first dose (data not shown). Both CD4 receptor saturation and CD4 receptor downmodulation had fully reversed by the end of the study at Day 68/69 (data not shown).

### PK Data Analysis

Nominal sample collection times and doses were used in the data analysis. Serum concentrations at or below the LOQ were treated as missing and were not used in the presentation or analysis of the PK data. For PK data calculations, Study Day 1 was converted to PK Day 0 to indicate the start of dosing (see Blood Sample Collection). The mean (±SD) PK parameters are presented in Table 5.

TABLE 5
Non-Compartmental PK Parameter Estimates (Mean ± SD)
Following IV Infusion Administration of Variant B, Variant C, or
Variant D to Baboons

| | Variant B | Variant C | Variant D |
|---|---|---|---|
| | 40 mg/kg IV Four | 40 mg/kg IV Four | 40 mg/kg IV Four |
| | Doses | Doses | Doses |
| PK Parameter | (n = 12) | (n = 12) | (n = 12) |
| AUCₐₗₗ | 30900 ± 8240 | 46100 ± 6090 | 60000 ± 11900 |
| (day · μg/mL) | | | |
| CL (mL/kg/ | 5.53 ± 1.46 | 3.53 ± 0.501 | 2.77 ± 0.615 |
| day) | | | |
| Vₛₛ (mL/kg) | 69.5 ± 18.1 | 58.0 ± 6.23 | 48.6 ± 9.71 |

AUCₐₗₗ = area under the serum concentration-time curve to last observation point;
CL = clearance;
PK = pharmacokinetic;
Vₛₛ = volume of distribution at steady state.

Noncompartmental analysis of the data indicated that CL was decreased by 36.2% in samples from animals given Variant C and 49.9% in samples from animals given Variant D compared with Variant B. These reduced CL values corresponded with an increase in half-life for the variants C and D compared with Variant B. It was expected that the half-life of Variant B would be concentration dependent because of the receptor-mediated CL and resulting nonlinear PK. The decreased CL of Variants C and D resulted in increased exposure (AUC) of 49.3% and 94.3% for Variant C and Variant D, respectively, when compared with Variant B. The decreased CL and increased AUC of both variants C and D were statistically significant compared with Variant B with p-values≦0.001 (calculated using the Tukey-Kramer adjustment for multiple testing). The results of a two-sided 95% confidence interval with Tukey-Kramer adjustment for multiple testing indicated that the CL of Variant D was reduced 38%-59% compared with Variant B. The volume of distribution ranges of the variants C and D were similar to Variant B (approximately 50-70 mL/kg), indicating that all three molecules remained mostly in the blood compartment. This result was consistent with prior observations that the 40 mg/kg dose saturates CD4-mediated CL.

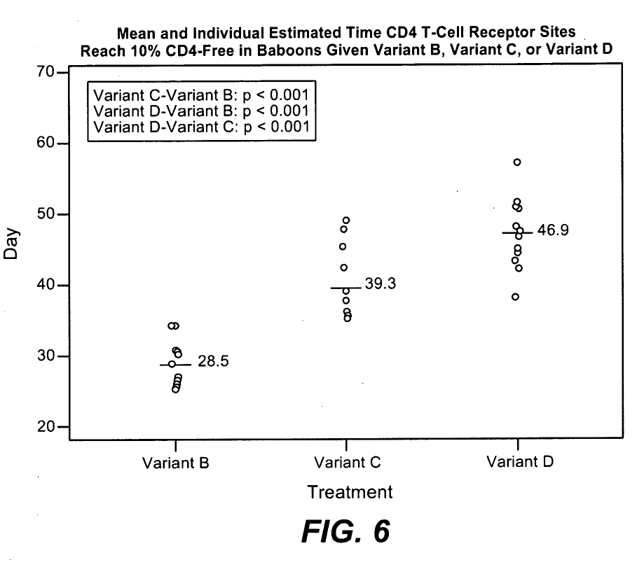

In addition, CD4 T-cell receptor occupation was evaluated by flow cytometry. The results are presented in FIG. 6. As early as 1 hour after the first dose administration of Variant B, Variant C, or Variant D, >98% of the CD4 T-cell receptors were occupied, compared with predose baseline measurements. CD4 T-cell receptor occupancy was not observed in the baboons in the vehicle group at any time. For each baboon, the available CD4 T-cell receptors were plotted over time and a cubic smoothing spline was fitted. These fitted lines were used to calculate the day on which the percentage of CD4-free sites crossed the 10% line, indicating that CD4 T-cell receptor occupancy was <90%. The average time at which animals given Variant B lost full CD4 T-cell receptor occupancy (<90%) was Day 28.6. For animals treated with Variant C, the average time at which animals lost full CD4 T-cell receptor occupancy was Day 39.3, and for Variant D, the average time at which animals lost full CD4 T-cell receptor occupancy was Day 46.9. These estimated times when less than 90% of the CD4 T-cell receptors were occupied were significantly different between the Variant B, Variant C, and Variant D-treated animals (FIG. 6).

These data indicate that Variants C and D, which were aglycosylated and had increased affinity for FcRn, had decreased CL compared with Variant B, which was only aglycosylated. These data thus show, perhaps for the first time, that antibodies engineered to both lack glycosylation and to have increased affinity for FcRn demonstrated increased serum half-life in vivo compared to their counterpart which only lacked glycosylation. Receptor-mediated CL should be the same for all variants tested because the affinity to CD4 is the same for Variants C and D as it is for Variant B and the extent of CD4 receptor saturation and downmodulation was similar between the molecules. The decreased CL can be attributed to the FcRn interaction and a more efficient recycling into circulation of Variants C and D compared with Variant B. The recycling resulted from increased binding of Variants C and D to FcRn in epithelial cells at low pH, following uptake of the antibodies by pinocytosis (see, e.g., Raghavan et al., Biochemistry 34:14649-57, 1995). The FcRn recycles the antibody into the serum instead of directing it to the lysosome for degradation because Variants C and D still have low affinity for FcRn at pH 7.4. In situations where CD4 is not saturated, CD4 receptor-mediated CL is thought to be the major elimination pathway. In those situations, the benefit of the reduced FcRn-mediated CL will likely be dose and concentration dependent. In addition, the data showed that CD4 receptor occupancy was prolonged in animals treated with Variants C and D compared to Variant B. The decreased clearance and thus, more prolonged exposure of Variants C and D, along with prolonged CD4 receptor occupancy, may be desirable by enabling less frequent dosing and/or lower doses and/or non-intravenous administration.

### Example 2

### In Vivo Administration of Non-depleting Anti-CD4 Variant D by Intravenous or Subcutaneous Routes

Variant D was administered to baboons by repeated intravenous (IV) or subcutaneous (SC) injection eight times at weekly intervals (8-week dosing period) and serum Variant D concentrations were determined. Sixty naive male and female baboons (Papio anubis) were divided into five dose groups (6/sex/group) and administered either control article (Variant D Vehicle) or test article (Variant D) once weekly for 8 weeks as indicated in Table 6 below. A total of 30 animals (3 males and 3 females from each of groups 1-5) underwent a 10-week recovery phase following the last dose.

TABLE 6
Study Design for In Vivo Administration of Variant D

| | | Dose | Dose | | Number |
|---|---|---|---|---|---|
| | Dose Level | Concentration | Volume | Dose | (Male/ |
| Group | (mg/kg) | (mg/mL) | (mL/kg) | Route | Female) |
| 1 | 0 (vehicle) | 0 | 0.5 | IV & SC | 6/6 |
| 2 | 5 | 10 | 0.5 | IV | 6/6 |
| 3 | 15 | 30 | 0.5 | IV | 6/6 |
| 4 | 50 | 100 | 0.5 | IV | 6/6 |
| 5 | 50 | 100 | 0.5 | SC | 6/6 |

### Intravenous Administration (Groups 1-4):

Intravenous injection in Group 1 was performed first followed by the subcutaneous injection. The site of dose administration was prepared prior to dose administration by shaving. Animals were dosed via a superficial vein on the arm or leg at a rate of 3-4 mL/min of continuous IV infusion. The butterfly infusion was primed so that no flush was required.

### Subcutaneous Injection (Groups 1 and 5):

The site of dose administration was prepared prior to dose administration by shaving and preparing the area. Test article was administered subcutaneously on the dorsal trunk (intrascapular area). The dose administration area was divided into approximate dosing quadrants using a permanent marker. The sites were labeled as SC-1, SC-2, SC-3, and SC-4. Dosing was rotated sequentially among the four numbered sites.

### General Blood Collection Procedure, Sample Processing and Variant D Serum Concentration Assay

Animals were sedated during all blood collections. Blood was collected by venipuncture from a peripheral vein. Approximately 1 mL of blood was collected from animals fasted overnight, then placed in a serum separator tube. Samples were collected for all animals on days 1, 8, 15, 22, 29, 36, 43, and 50. Samples from recovery animals were collected on days 60, 64, 67, 71, 78, 85, 92, 99, 107, and 120/121.

Samples were allowed to clot at room temperature for 20-60 minutes. Clotted samples were centrifuged within 1 hour of the collection time at a relative centrifugal force of 1,500-2,000×g for 10-15 minutes at 2° C.-8° C. Serum was separated from the samples within approximately 20 minutes of the end of centrifugation and transferred into prelabeled Eppendorf tubes (approximately 500 μL). The tubes were held on dry ice unless stored immediately in a freezer set to maintain a temperature within a range of −60° C. to −86° C. until samples were assayed.

The concentration of Variant D in serum samples was determined using an ELISA assay (see above). The assay limit of quantitation (LOQ) was 0.008 μg/mL in neat serum.

### Flow Cytometry of Peripheral Blood and Tissues

Blood samples for flow cytometry were collected from all animals twice during acclimation (Days-12 and -6), prior to dosing on Days 1, 8, 15, 22, 29, 36, 43, and 50, and weekly during the recovery period at same time that hematology and toxicokinetic samples were collected. No samples were collected during Week 9 of recovery. Samples were collected on the day of necropsy at Week 10, prior to administration of euthanasia solution. Representative sections of the spleen, mesenteric lymph nodes, and mandibular lymph nodes (right) from each animal were collected at necropsy and cell suspensions were prepared for flow cytometry analysis.

Flow cytometric analyses of peripheral-blood samples and lymphoid organs (lymph nodes and spleen) showed that Variant D administration resulted in a dose-dependent saturation of peripheral blood and tissue T-cell CD4 receptors as well as CD4 receptor downmodulation. At 5, 15, and 50 mg/kg IV and 50 mg/kg SC of Variant D, peripheral-blood CD4 receptor saturation of 59%, 85%, 95%, and 92%, respectively, was observed 3 days after the first dose. The peripheral T lymphocyte CD4 receptor saturation was paralleled by a 20-40% downmodulation of CD4 expression; receptor saturation and downmodulation resolved completely in a dose-dependent manner during the recovery phase. In the lymphoid tissues examined, approximately 20% CD4 T-cell saturation was observed in all Variant D-administered groups at terminal necropsy and approximately 20%-40% downmodulation of CD4 T-cell receptor expression was observed at terminal necropsy, which returned to control levels by recovery necropsy. No CD4 receptor occupancy on tissue T lymphocytes was observed at recovery necropsy.

Mean percentages of several peripheral blood and tissue lymphocyte subsets of Variant D-administered groups decreased when compared with those of control animals. This finding was observed for both T-lymphocyte subsets (CD3, CD3 CD4, CD4 CD45RA, and CD4 CD45RA-) as well as B-lymphocyte subsets (CD20, CD20 CD21). However, the absolute cell counts of these subsets did not fall outside the normal range as established by vehicle control and the predose values of all groups. In the tissues, large variability was observed in the absolute cell counts within each group. In general, Variant D administration at 5, 15, or 50 mg/kg IV or at 50 mg/kg SC did not induce any substantive changes in T- and B-cell lymphocyte subsets or natural killer (NK) cells in the peripheral blood or the lymph node and spleen tissues.

In summary, Variant D administration was well tolerated at doses up to 50 mg/kg by baboons following IV or SC administration of 8 weekly doses. Variant D administration produced the pharmacologic effects of CD4+ T-cell receptor saturation and downmodulation without T-cell depletion, results that are consistent with a non-depleting anti-CD4 antibody.

### Variant D Concentrations in Serum

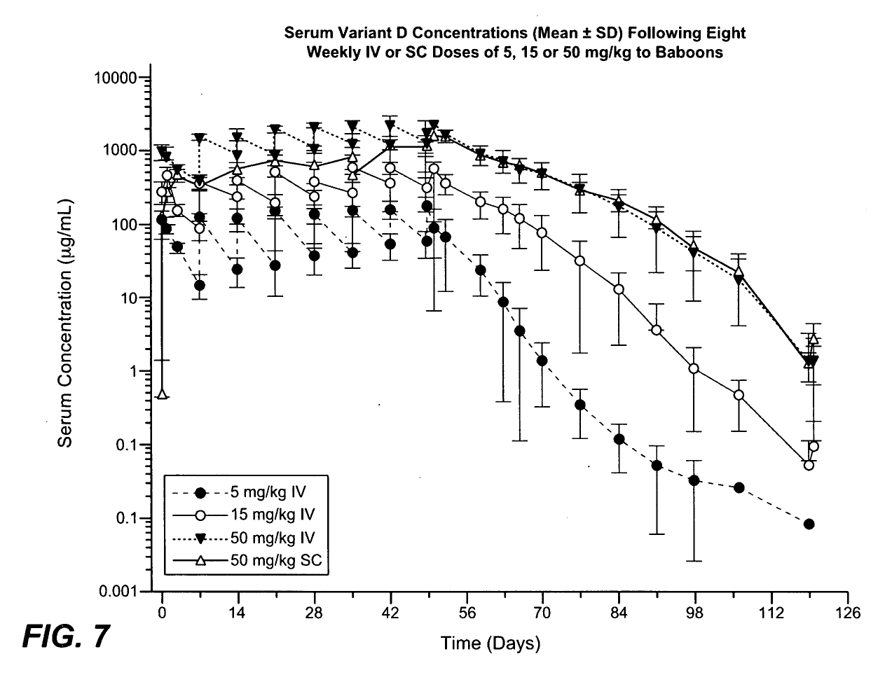

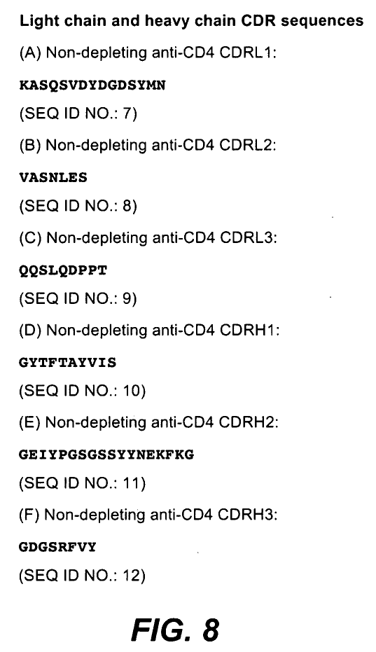

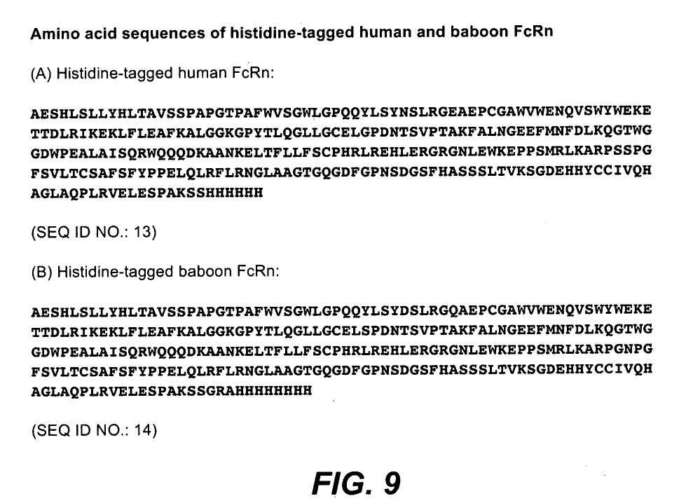

Serum Variant D concentration-time profiles over time following repeated IV infusions of 5, 15, and 50 mg/kg or SC doses of 50 mg/kg to baboons weekly for a total of eight doses (Groups 2, 3, 4, and 5, respectively) are presented in FIG. 7. Variant D in serum showed a biphasic disposition, with a fast distribution phase followed by a prolonged elimination phase. The terminal phase of the profiles also appeared nonlinear characterized by decreasing slope with increasing dose.

The estimated TK parameters for Variant D for the 5, 15, and 50 mg/kg IV and 50 mg/kg SC dose groups (Groups 2, 3, 4, and 5 respectively) are presented in Table 7. Following eight doses of Variant D (see Table 7), the exposure, defined as area under the serum concentration-time curve (AUCₗₐₛₜ), increased in a non-dose proportional manner over the dose range tested. The dose-normalized AUCₗₐₛₜ values for the 5, 15, and 50 mg/kg IV dose groups were 871, 1510, and 1920 day ·μg/mL/(mg/kg), respectively. Lack of dose proportionality in the AUC is consistent with nonlinear pharmacokinetics of Variant D due to CD4-mediated elimination, which is saturated at higher doses. The observed Cmin values also remained consistent throughout the dosing phase within each dose group (data not shown). The TK profiles also appeared similar between sexes. There was moderate accumulation following eight weekly doses of Variant D with an RCₘᵢₙ that ranged from 3 to 4. Bioavailability of Variant D following SC administration of 50 mg/kg was 67.2%. The mean (±SD) TK parameters for each dose group are presented in Table 7.

TABLE 7
Non-Compartmental TK Parameter Estimates (Mean ± SD) Following
Eight Weekly Doses of 5, 15, or 50 mg/kg to Baboons

| | 5 mg/kg IV | 15 mg/kg IV | 50 mg/kg IV | 50 mg/kg SC |
|---|---|---|---|---|
| TK Parameter | (n = 12) | (n = 12) | (n = 12) | (n = 12) |
| AUC₀₋₇ (day · μg/mL) | 375 ± 51.6 | 1240 ± 241 | 4430 ± 413 | 2880 ± 456 |
| AUC₀₋₅₂ (day · μg/mL) | 4570 ± 923 | 18700 ± 3140 | 74900 ± 9170 | 39600 ± 5500 |
| AUCₗₐₛₜ (day · μg/mL) | 4360 ± 591 | 22700 ± 4540 | 96200 ± 16000 | 64700 ± 8320 |
| AUC₀₋₇/Dose | 75.1 ± 10.3 | 82.6 ± 16.0 | 88.7 ± 8.26 | 57.5 ± 9.13 |
| (day · μg/mL/[mg/kg]) | | | | |
| AUC₀₋₅₂/Dose | 915 ± 185 | 1250 ± 209 | 1500 ± 183 | 791 ± 110 |
| (day · μg/mL/[mg/kg]) | | | | |
| AUCₗₐₛₜ/Dose | 871 ± 118 | 1510 ± 303 | 1920 ± 321 | 1290 ± 166 |
| (day · μg/mL/[mg/kg]) | | | | |
| Obs Cₘₐₓ (μg/mL) | 204 ± 41.4 | 777 ± 133 | 2610 ± 427 | 1640 ± 252 |
| tₘₐₓ (day) | 48.7 ± 2.11 | 44.0 ± 8.15 | 44.0 ± 5.53 | 49.8 ± 2.62 |
| Obs Cₘᵢₙ (μg/mL) | 14.9 | 87.5 | 399 | 330 |
| RCₘᵢₙ | 4.01 ± 0.920 | 3.57 ± 0.856 | 3.06 ± 0.724 | 3.23 ± 0.531 |
| F (%) | NA | NA | NA | 67.2 |

AUC₀₋₇ = area under the serum concentration-time curve from time 0 to Study Day 8 (TK Day 7);
AUC₀₋₅₂ = area under the serum concentration-time curve from time 0 to Study Day 53 (TK Day 52);
AUCₗₐₛₜ = area under the serum concentration-time curve (calculated for the recovery group only, n = 6);
AUC₀₋₇/Dose = area under the serum concentration-time curve from time 0 to Study Day 8 (TK Day 7) normalized by nominal dose;
AUC₀₋₅₂/Dose = area under the serum concentration-time curve from time 0 to Study Day 53 (TK Day 52) normalized by nominal dose;
AUCₗₐₛₜ/Dose = area under the serum concentration versus time curve between TK Day 0 and the last observed concentration (calculated using only the recovery group animals) normalized by nominal dose;
F = bioavailability;
(AUCₗₐₛₜ SC) ÷ (AUCₗₐₛₜ IV) calculated using the data from the recovery group animals only;
obs Cₘₐₓ = maximum observed concentration;
obs Cₘᵢₙ = minimum observed concentration during the dosing period;
RCₘᵢₙ = accumulation ratio (serum trough concentration before last dose, Study Day 50 [TK Day 49] divided by the serum trough concentration before second dose, Study Day 8 [TK Day 7]);
tₘₐₓ = time of maximum observed concentration.

In summary, the data above show that the toxicokinetic (TK) profiles appeared nonlinear following IV administration of 5, 15, or 50 mg/kg Variant D in baboons. Variant D exposure, or area under the serum concentration-time curve (AUC), increased non-dose proportionally over the dose range examined (5-50 mg/kg), consistent with nonlinear pharmacokinetics of Variant D due to CD4-mediated elimination. The observed Cₘᵢₙ values remained consistent throughout the dosing phase within each of the groups. The TK profiles also appeared similar between sexes. There was moderate accumulation following weekly dosing of Variant D with an RCₘᵢₙ that ranged from 3 to 4. Bioavailability of Variant D following SC administration of 50 mg/kg was 67.2%.

### CONCLUSION

The data presented and discussed in the examples above suggests that Variant D will have improved safety and will enable a more desirable dosing regimen for the treatment of autoimmune diseases compared to anti-CD4 antibodies described previously in the art, including previously-described non-depleting anti-CD4 antibodies. The concern with targeting T cells has been reduction or depletion that could lead to immune suppression. Lack of Fcγ interaction with Variant D as described above, via the substitution at position 297, may provide improved safety because of the lack of T-cell depletion and potential for reduced infusion reactions, both of which are mediated through the Fcγ receptors.

In addition, because of the rapid clearance resulting from CD4-mediated elimination, frequent dosing may be required to maintain CD4 downmodulation and saturation. Variant D, which has an amino acid substitution at position 434, showed increased FcRn binding and had a 50% reduced clearance in baboons compared with the wild-type antibody without the substitution at that position. This more prolonged exposure of Variant D may enable less frequent dosing and/or lower doses and/or alternative routes of administration, e.g., subcutaneous, than had previously been possible with anti-CD4 antibodies described previously in the art. Accordingly, the data support a phase I clinical study in an exemplary autoimmune disease, rheumatoid arthritis, as described below.

### Example 3

### A Phase I Study of a Non-depleting Anti-CD4 Antibody (Variant D) Administered by Intravenous or Subcutaneous Routes in Patients with Rheumatoid Arthritis

### Study Design

This is a Phase I multicenter study that will be conducted in the United States and consists of a double-blind (investigator and patient), placebo-controlled, single ascending-dose (SAD) stage, followed by a double-blind (investigator and patient), placebo-controlled multiple ascending-dose (MAD) stage using different patients from those in the SAD stage. The MAD stage population reflects the patient population most likely to receive Variant D in future studies. The study will be conducted in approximately 65 adult patients between 18 and 80 years old who have RA. Patients enrolled in the SAD stage will have a diagnosis of RA without pre-specified disease activity. Patients enrolled in the MAD stage will have mild to moderate disease activity, defined as a tender and swollen joint count of ≧3 and inadequate response to at least one biologic agent. The single-does study cohorts are summarized in Table 8 below.

TABLE 8
Single-Dose Study Cohorts

| Co- | | Dose | Total No. | Route of | No. of Patients^{a} | |
|---|---|---|---|---|---|---|
| hort | Stage | (mg/kg) | of Doses | Administration | Variant D | Placebo |
| A | SAD | 0.3 | 1 | intravenous | 4 | 1 |
| B | SAD | 1.0 | 1 | intravenous | 4 | 1 |
| C | SAD | 1.0 | 1 | subcutaneous | 4 | 1 |
| D | SAD | 3.5 | 1 | intravenous | 4 | 1 |
| E | SAD | 3.5 | 1 | subcutaneous | 4 | 1 |
| F | SAD | 7.0 | 1 | intravenous | 4 | 1 |

^{a}An additional 5 patients (in 4:1 ratio Variant D to placebo) may be enrolled if necessary, as specified in the dose-escalation rules (described below).

### Single Ascending-Dose Stage

Following screening, 30 patients will be sequentially enrolled into six cohorts of 5 patients each (treatment allocation of 4:1 Variant D to placebo; Cohorts A-F; see table above) with four intravenous (IV) dose levels (0.3, 1.0, 3.5, and 7.0 mg/kg) and two subcutaneous (SC) dose levels (1.0 and 3.5 mg/kg).

The first dose cohort (Cohort A; 0.3 mg/kg IV) will be the first-in-human dosing of Variant D; therefore, no more than 1 patient in the initial cohort will receive study drug (Variant D or placebo) on any single day. Safety data from Cohort A will be reviewed after at least 14 days of follow-up have been completed for all patients. If Variant D demonstrates acceptable safety in Cohort A, according to the pre-specified dose-escalation rules, the next 5 patients will be enrolled in Cohort B (1.0 mg/kg IV). Enrollment in Cohort C (1.0 mg/kg SC) will occur immediately upon the enrollment of patients in Cohort B. After at least 14 days of follow-up have been completed for all patients in Cohort B, a safety review will occur and enrollment may begin in Cohort D (3.5 mg/kg IV) after Cohort C has been fully enrolled. Enrollment in Cohort E (3.5 mg/kg SC) will occur immediately upon the enrollment of Cohort D. After at least 14 days of follow-up have been completed for all patients in Cohort D, a safety review will occur and enrollment may begin in Cohort F (7.0 mg/kg IV) after Cohort E has been fully enrolled.

A minimum of 5 patients and a maximum of 10 patients will be enrolled in each IV SAD cohort. If 1 of the 4 patients treated with Variant D develops a dose-limiting toxicity (DLT), an additional 5 patients (in a 4:1 ratio Variant D to placebo) will be enrolled in that cohort. If only 1 of the 8 Variant D-treated patients experiences a DLT, then dose escalation will occur. If more than 1 Variant D-treated patient experiences a DLT, dose escalation will be suspended and the available safety data will be reviewed. A total of 5 patients will be enrolled in each SC cohort. If more than 1 patient treated with Variant D in an SC cohort experiences a DLT, enrollment will be suspended and the available safety data will be reviewed.

Potential acute toxicities, such as hypersensitivity reactions, infusion reactions, or serum sickness-type reactions, injection-site reactions, rash, etc., may occur and are likely to have resolved during the 14-day observation period. Blood samples will be collected for PK, PD, and anti-therapeutic antibody (ATA) assessments. All patients in the SAD stage will undergo safety follow-up through Day 36 (5 weeks after dosing). All available safety data in the SAD stage through at least 14 days of follow-up for all patients in Cohort E will be reviewed prior to the initiation of the MAD stage. Safety data will include white blood cell count and types by complete blood count (CBC) and differential and T-cell subsets by flow cytometry.

### Multiple Ascending-Dose Stage

The objective of this stage is to characterize the safety and PK/PD properties of Variant D given weekly for eight doses over the proposed dose range (1.5 and 3.5 mg/kg SC and 5.0 mg/kg IV). All available safety data in the SAD stage through at least 14 days of follow-up for all patients in Cohort E will be reviewed prior to the initiation of the MAD stage. Additionally, at least 14 days of safety follow-up for all patients in Cohort F will be reviewed prior to enrollment of Cohort H of the MAD stage. Safety data will include white blood cell count and types by complete blood count (CBC) and differential and T-cell subsets by flow cytometry. Ongoing review of the safety and PK/PD data will be performed by the Sponsor's Medical Monitor, the drug safety scientist, and the biostatistician who will not be blinded to treatment assignment and dose.

Following screening, a total of 35 patients will be enrolled in three cohorts (G, H, and I; see table 9 below) in a sequential manner from Cohort G to I. Cohorts G (1.5 mg/kg SC) and H (3.5 mg/kg SC) will consist of 12 patients randomized to receive Variant D and 3 to placebo. Cohort I (5.0 mg/kg IV) will consist of 4 patients randomized to receive Variant D and 1 to placebo. The patients enrolled in the MAD stage will be distinct from those in the SAD stage; therefore, patients dosed in the SAD stage will not be eligible for enrollment in the MAD stage. Study drug will be administered to patients subcutaneously or intravenously every week for a total of eight doses. Blood samples will be collected for PK, PD, and ATA assessments. All patients in the MAD stage will undergo safety follow-up through Day 113. This schedule estimates that approximately 16 patients will be treated at or above the target dose of Variant D based on predicted exposure (3.5 mg/kg SC or 5 mg/kg IV) and that a total of approximately 7 patients in the entire MAD stage will receive placebo.

Enrollment in Cohort H (n=15; 3.5 mg/kg SC) will occur after Cohort G (n=15; 1.5 mg/kg SC) has been fully enrolled and at least 6 patients have completed 2 weeks of treatment and safety data for patients in Cohort E of the SAD stage have been reviewed. Enrollment in Cohort I (n=5; 5.0 mg/kg IV) will occur immediately upon full enrollment of Cohort H. The dose levels in the MAD stage may be modified if a maximum tolerate dose is observed in the SAD stage.

TABLE 9
Multiple-Dose Study Cohorts

| | | | Total No. | | | |
|---|---|---|---|---|---|---|
| Co- | | Dose | of Weekly | Route of | No. of Patients | |
| hort | Stage | (mg/kg) | Doses | Administration | Variant D | Placebo |
| G | MAD | 1.5 | 8 | subcutaneous | 12 | 3 |
| H | MAD | 3.5 | 8 | subcutaneous | 12 | 3 |
| I | MAD | 5.0 | 8 | intravenous | 4 | 1 |

For both the SAD and MAD stages of the study, eligible patients will be randomized within 4 weeks of screening. The incidence and nature of adverse events, serious adverse events, and laboratory abnormalities will be assessed.

### Inclusion Criteria

For the SAD stage, the target candidate for the SAD portion of the study is a patient with RA who may be on a stable regimen of anti-rheumatic therapy (see Table 10).

For the MAD stage, the target candidate for the MAD portion of the study is a patient with RA who currently has at least a minimal amount of disease activity and has had an inadequate response to at least one biologic therapy. These patients will have evidence of disease activity with at least 3 tender and swollen joints (e.g., the temperomandibular joint, stemoclavicular joint, acromioclavicular joint, shoulders, elbows, wrists, interphalangeal joints, metacarpophalangeal joints, hips, knees, ankles, and/or metatarsals) and will be on a stable therapy as specified in Table 10. Failure of at least one biologic agent is defined as lack of or loss of response (at doses indicated below) or intolerance. Exemplary biologic agents include anti-TNF agents such as adalimumab (40 mg administered every other week for at least 3 months); etanercept (50 mg administered weekly [or 25 mg administered twice a week] for at least 3 months); and infliximab (administration of >3 mg/kg with at least four infusions). Other exemplary biologic agents include rituximab (up to 2×1000 mg IV administered) and ocrelizumab (up to 2×1000 mg IV administered).

### Exclusion Criteria

Patients who meet any of the following criteria are not eligible for enrollment in either the SAD or MAD stage:

Female patients who are pregnant, plan to become pregnant during the study, or are breastfeeding

Clinically significant abnormal laboratory values or ECG (e.g., creatinine>1.5× the upper limit of normal [ULN], transaminases elevated>2.5× the ULN, or abnormalities in synthetic function tests judged by the investigator to be clinically significant)

History of anaphylactic reactions

Positive hepatitis C antibody or hepatitis B surface antigen

Positive serology for human immunodeficiency virus (HIV) by quantitative polymerase chain reaction

Positive (not just reactive) purified protein derivative (PPD; >5-mm wheal) without evidence of treatment for tuberculosis

A history of an autoimmune disease other than RA (other than secondary Sjögren's syndrome)

Significant systemic involvement of RA, including vasculitis, pulmonary fibrosis, or Felty's syndrome

Malignancy, or prior malignancy, other than non-melanoma skin cancer or cervical carcinoma in situ that has been resected

Administration of a live, attenuated vaccine within 1 month before dosing with Variant D, or anticipation that such a live attenuated vaccine will be required during the study or within 60 days after the last dose. Influenza vaccination should be performed during influenza season only (approximately October to March). Patients must not receive live attenuated influenza vaccine (e.g., FLUMIST®) within 30 days prior to randomization or at any time during the study.

History of treatment with any T cell-directed therapy (e.g., abatacept, keliximab, and ibalizumab)

Concomitant therapy with a biologic agent

Previous exposure to Variant D or treatment with any investigational agent 12 weeks or 5 half-lives of the investigational agent (whichever is longer) prior to Day 1

### Study Treatment

The study drug, a non-depleting anti-CD4 monoclonal antibody, Variant D, is manufactured and supplied by Genentech, along with the placebo. Variant D is produced in Chinese hamster ovary cells, purified, and subjected to quality control procedures. Both the drug product and placebo are sterile, preservative-free liquids intended for both SC and IV administration. The placebo is identical in composition to the Variant D drug product but does not contain Variant D antibody. Phase I clinical trials will be conducted with a single-use formulation administered to patients by IV infusion or SC injection. Active study drug or placebo for IV administration will be provided as a parenteral formulation in a 3-cc USP/Ph. Eur. Type 1 glass vial with a 13-mm fluoro-resin laminated stopper and capped with a 13-mm aluminum seal with a plastic flip-off cap.

The Variant D dose (in milligrams per kilogram) and route of administration will be determined by cohort assignation and patient's body weight at screening. The patient will be randomized through an interactive voice response system (IVRS) to receive active study drug or placebo.

For patients assigned to receive IV injections, study drug will be administered intravenously, after dilution in normal saline (0.9%), by IV infusion from a saline bag using an infusion pump. The volume of study drug to be given will be calculated for each patient. For patients assigned to receive SC injections, study drug will be administered subcutaneously in the deltoid region of the right or left arm. Alternatively, the injections can be administered in the thigh if medically significant reasons preclude administration in the deltoid region.

### Concomitant Therapy and Clinical Practice

All patients in the study will be permitted to continue treatment with approved stable doses of corticosteroids, disease-modifying anti-rheumatic drugs, and non-steroidal anti-inflammatory drugs. Doses of concomitant medications will be considered stable if the dose level and frequency have not been adjusted for at least the time specified in Table 10 below. A record of any other concomitant medication administered to patients during study participation will be maintained during the study for each study participant. Concomitant therapy includes any prescription medications or over-the-counter preparations used by a patient between the 30 days preceding the screening evaluation and the end of study visit. Patients who use oral contraceptives, hormone-replacement therapy, or other maintenance therapy should continue their use.

TABLE 10
Anti-Rheumatic Therapies

| | Minimum Length | |
|---|---|---|
| | of Stable Regimen | Maximum |
| | before Randomization | Allowed Dosage |
| DMARDS:^{a} | | |
| Methotrexate^{b} | 4 weeks | 25 mg weekly |
| Leflunomide^{b} | 8 weeks | 20 mg daily |
| Sulfasalazine | 6 weeks | 3 g daily |
| Hydroxychloroquine | 8 weeks | 400 mg daily |
| Other Agents: | | |
| Prednisone or equivalent | 4 weeks | 10 mg/day oral |
| NSAIDs (including Cox-2 | 2 weeks | Per to prescribing |
| selective agents [coxibs]) | | information |

DMARD = Disease-Modifying Anti-Rheumatic Drug; NSAID = Non-Steroidal Anti-Inflammatory Drug
^{a}The only DMARDs allowed are those listed above. No change in DMARD or dose is allowed for at least the period indicated above. Patients may be on a combination of two DMARD therapies, as long as the combination regimen (including the dose of each individual drug) has been stable for at least 4 weeks prior to randomization, is well-tolerated, is not associated with significant laboratory abnormalities, and, in the opinion of the investigator, will not pose additional risk to the patient or confound the interpretation of the study endpoint data.
^{b}The combination of methotrexate and leflunomide is not permitted prior to or during the study.

### Outcome Measures

### Safety Outcome Measures

The primary safety outcome measures of the study are the safety and tolerability of Variant D in both the SAD and MAD stages. Safety will be assessed by the incidence of adverse events, graded according to the National Cancer Institute Common Toxicity Criteria for Adverse Events (NCI CTCAE), v3.0.

### Pharmacokinetic and Pharmacodynamic Outcome Measures

The following PK parameters will be derived from the serum concentration-time profile of Variant D following study drug administration:

Maximum serum concentration (Cₘₐₓ)

Clearance (CL) or apparent CL (CL/F) for drugs given subcutaneously

Volume of distribution (V) or apparent volume of distribution (V/F) for drugs given subcutaneously

Total exposure (area under the concentration-time curve [AUC])

Dose proportionality

SC bioavailability (F)

Incidence of anti-therapeutic antibodies (ATAs) using samples obtained at multiple timepoints before and after dosing for each patient

The PD parameter that will be assessed following Variant D administration is CD4 expression and occupancy on peripheral blood T cells by flow cytometry.

### Results of the Single Ascending-Dose Study

### Pharmacokinetic Characterization

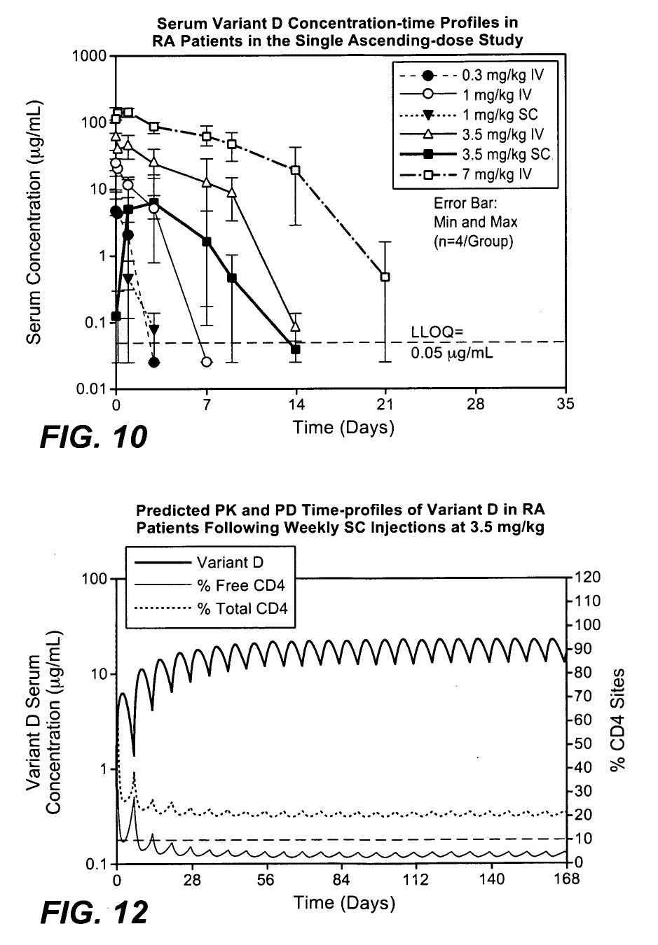

Serum Variant D concentration-time profiles following single IV infusions of 0.3, 1.0, 3.5 and 7.0 mg/kg or SC doses of 1.0 mg/kg and 3.5 mg/kg to RA patients are shown in FIG. 10. The pharmacokinetic profile appeared nonlinear characterized by increasing slope with time or decreasing dose. The estimated PK parameters for Variant D for all dose groups are presented in Table 11. Following a single dose of Variant D, the total exposure, defined as area under the serum concentration-time curve (AUCₐₗₗ), increased in a non-dose proportional manner over the dose range tested. The lack of dose proportionality in the AUC is consistent with nonlinear pharmacokinetics of Variant D due to CD4-mediated elimination, which is saturated at higher doses.

TABLE 11
Non-compartmental PK parameter estimates (Mean ± SD) following single doses of
Variant D to RA patients

| | IV | | | | SC | |
|---|---|---|---|---|---|---|
| | 0.3 mg/kg | 1.0 mg/kg | 3.5 mg/kg | 7.0 mg/kg | 1.0 mg/kg | 3.5 mg/kg |
| PK Parameter | (n = 4) | (n = 4) | (n = 4) | (n = 4) | (n = 4) | (n = 4) |
| Obs Cₘₐₓ | 4.72 ± 4.14 | 21.8 ± 5.11 | 47.5 ± 21.3 | 147 ± 18.7 | 0.240 ± 0.406 | 6.38 ± 6.57 |
| (μg/mL) | | | | | | |
| AUCₐₗₗ | 4.70 ± 4.71 | 39.9 ± 11.7 | 201 ± 151 | 994 ± 288 | 0.387 ± 0.671 | 33.0 ± 36.5 |
| (day · μg/mL) | | | | | | |
| AUCₐₗₗ/Dose | 15.7 ± 15.7 | 39.9 ± 11.7 | 57.3 ± 43.2 | 142 ± 41.1 | 0.387 ± 0.671 | 9.42 ± 10.4 |
| (day · μg/mL/ | | | | | | |
| [mg/kg]) | | | | | | |

Obs Cₘₐₓ = maximum observed concentration;
AUCₐₗₗ = area under the serum concentration-time curve from Day 0 to the last observed concentration;
AUCₐₗₗ/Dose = area under the serum concentration versus time curve from Day 0 to the last observed concentration normalized by nominal dose.

### Pharmacodynamic Responses

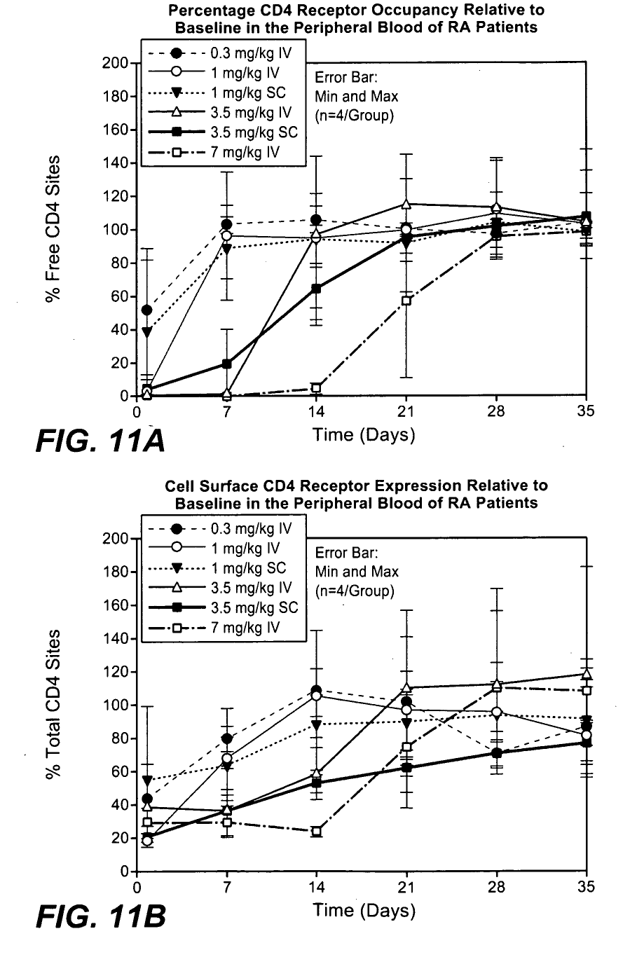

Flow cytometry analysis was performed to evaluate the PD effects of Variant D. Blood samples were collected pre-dose, and at Day 0, 7, 14, 21, 28 and 35 after single dose administration of Variant D and analyzed for lymphocyte subsets using standard clinical flow cytometry procedures. Specifically, the PD responses measured were CD4 receptor occupancy and CD4 receptor expression on peripheral blood T cells. The mean data of single dose cohorts is plotted in FIGS. 11A and 11B. A dose dependent occupancy of CD4 T cells (or decrease in % Free CD4 sites) was observed after a single dose administration of 0.3, 1.0, 3.5 or 7.0 mg/kg IV or 1.0 mg/kg and 3.5 mg/kg SC of variant D (FIG. 11A). Full CD4 occupancy was observed 24 hr after dose administration of Variant D in the 1.0, 3.5, 7.7 mg/kg IV and 3.5 mg/kg SC dose cohorts. Moreover, CD4 occupancy recovered in a dose dependent manner, with the 7.0 mg/kg IV dose cohort recovering last, after Day 14, compared with the other dose cohorts. Cell surface expression of CD4 on peripheral blood T cells was also assessed using a non-blocking anti-CD4 Ab. Similar dose dependent trends were observed with CD4 expression as with CD4 occupancy after single dose administration of Variant D (FIG. 11B). Maximum CD4 down-modulation was 80% of baseline, which was observed until Day 14 in the 7.0 mg/kg IV dose cohort.

In addition to CD4 occupancy and expression, total numbers of T-lymphocytes, CD4 T cells, CD8 T cells, total B cells, monocytes and NK cells were also determined by flow cytometry. Lymphocyte counts remained stable over the course of the study (36 days) after a single dose administration of 0.3, 1.0, 3.5 or 7.0 mg/kg IV and 1.0 mg/kg or 3.5 mg/kg SC of Variant D (data not shown). These results thus demonstrate that Variant D does not deplete CD4 T cells.

Projected target dosing regimen in humans

To predict the Variant D serum concentration and PD time-profiles following multiple injections in human, a receptor-mediated PK/PD model characterizing the relationship between serum anti-CD4 concentration and total and free CD4 expression was simultaneously fitted to the PK/PD data from the single-ascending dose study of Variant D. We modified the model based on TRX1 Phase I data as reported in Ng et al., Pharm. Research 23:95-103, 2006. In this model, the plasma drug concentration is assumed to be eliminated by both non-specific elimination (Kₑₗ) and specific receptor-mediated endocytosis. Receptor-mediated endocytosis was modeled as an interaction with free CD4 receptor (R_{f}) to form a drug-receptor complex (X_{R}) via reversible (Kₒₙ and Koff) binding, followed by cellular internalization (Kᵢₙₜ). A tissue compartment with linear first-order distribution processes (K_{ct} and K_{tc}) was used to account for non-specific drug binding or distribution. To simulate subcutaneous doses, the model described by Ng et al. was expanded to include the dynamics of subcutaneous absorption, characterized by the rate of absorption (Kₐ) and bioavailability (F). The updated differential equations that describe the model are shown below:

dXsc/dt=−KaXsc

dXc/dt=KaFXsc+R0−(Kel+Kct)Xc+KtcXt−(Kon(Xc/Vc)Rf−KoffXR)Vc

dXt/dt=KctXc−KtcXt

dRf/dt=Ksyn−KdegRf−Kon(Xc/Vc)Rf+KoffXR

dXR/dt=Kon(Xc/Vc)Rf−KoffXR−KintXR

where Xsc, Xc and Xt are the amount of free antibody in the subcutaneous, central and tissue compartments, respectively; Rf and XR are the free CD4 and antibody-CD4 complex concentrations, respectively.

This model was simultaneously fitted to the mean PK and PD data using ADAPT 5, version 5.0.28 (University of Southern California, available at the URL (HTTP protocol) bmsr.usc.edu) to identify Kₑₗ, Kₐ, and F while keeping the other parameters fixed at the published values for TRX1. The fitted parameter values are shown in Table 12.

TABLE 12
Estimated Model Parameters Based on the fit of Variant D
Single-Ascending Dose PKPD Data

| | | Model |
|---|---|---|
| Parameter | Description | Estimate |
| Kₑₗ (Day⁻¹) | first-order nonspecific elimination rate | 0.0654 |
| | constant from the central compartment | |
| K_{ct} (Day⁻¹) | first-order distribution rate constant | 0.649 |
| | from central compartment to tissues | |
| K_{tc} (Day⁻¹) | first-order distribution rate constant | 0.874 |
| | from tissues to central compartment | |
| V_{c} (mL · kg⁻¹) | volume of distribution in the central | 41.7 |
| | compartment | |
| K_{deg} (Day⁻¹) | first-order elimination rate constant | 0.694 |
| | of free CD4 receptor | |
| Kₒₙ (nM · Day⁻¹) | association rate constant | 0.753 |
| K_{off} (Day⁻¹) | dissociation rate constant | 14.6 |
| Kᵢₙₜ (Day⁻¹) | first-order internalization/degradation | 3.93 |
| | rate constant of CD4 receptor complex | |
| K_{syn} (nM · Day⁻¹) | zero-order synthesis rate constant of | 38.1 |
| | free CD4 receptor | |
| Kₐ (Day⁻¹) | rate of absorption | 0.354 |
| F (%) | bioavailability | 58.3 |

Model simulation based on the fitted parameters illustrated that 3.5 mg/kg weekly subcutaneous doses are predicted to maintain over 90% CD4 saturation in peripheral blood at steady state (FIG. 12). As shown in FIG. 12, the Variant D serum concentration predicted to maintain such>90% CD4 saturation is 6 μg/ml. Accordingly, this data supports a target dose level of ˜3.5 mg/kg or a flat dose of ˜250 mg (assuming average body weight of ˜70 kg) once every week for Variant D delivered subcutaneously in order to achieve adequate CD4 saturation in peripheral blood in human. Given the variability of body weight and response in a patient population, a range of flat doses between 150 mg and 350 mg can be used in the clinic, taking into account the antibody concentration typical in a pharmaceutical formulation and feasible volumes that can typically be administered subcutaneously.

### Clinical Results

A total of 30 patients (five patients in each of six cohorts as described above) in the single ascending-dose study were evaluated according to the schedule and parameters described above. Administration of Variant D was safe and well tolerated in doses between 0.3 mg/kg and 7.0 mg/kg IV and 1.0 mg/kg and 3.5 mg/kg SC. No serious adverse events or dose-limiting toxicities were observed in any patient who received Variant D. In addition, there were no infusion reactions and no evidence of cytokine release syndrome (IL-1B, IL-2, IL-4, IL-5, IL-6, IL-13, IFNγ). Significantly, no drug-related rash was observed in any patient who received Variant D. While no clinical activity signal was observed after single doses, this was not unexpected due to the design of the study for the purpose of assessing PK/PD parameters and safety. The PK/PD parameters and safety profile reported here support further clinical studies.

### Example 4

### Variant D Inhibits Human Th1 and Th17 CD4+ Cells in a Mixed Lymphocyte Reaction

Upon productive stimulation, CD4+ T cells proliferate and differentiate to become polarized in their function and can be classified into subsets based on the profile of cytokines they produce. Until recently, Th1 CD4+ T cells, characterized by their secretion of Interferon-gamma (IFN-γ), have been considered significant contributors to autoimmune pathologies associated with several diseases such as RA, MS, and IBD. More recently, a newly identified subset of CD4+ T cells, designated Th17 cells based on their production of interleukin-17 (IL-17), has been implicated as a primary driver of pathogenesis in RA, MS, and SLE. (Garrett-Sinha et al., Curr. Op. Rheum. 20:519-525 (2008); Hsu et al., Nature Immunol. 9:166-175 (2008); Wong et al., Clin. Immunol. 127:385-393 (2008); Jacob et al., J. Immunol. 182:2532-2541 (2009)). In addition to a direct role in pathogenesis, IL-17 secretion by Th17 cells has been shown to contribute to germinal center formation and to synergize with BAFF to enhance B cell survival and differentiation to antibody-secreting cells in lupus. To determine the extent to which Th1 and Th17 T cell subsets are dependent on CD4 co-receptor function for their activation, we assessed each subset for proliferative capacity in a mixed lymphocyte reaction under conditions of increasing concentrations of Variant D or control antibody as described further below.

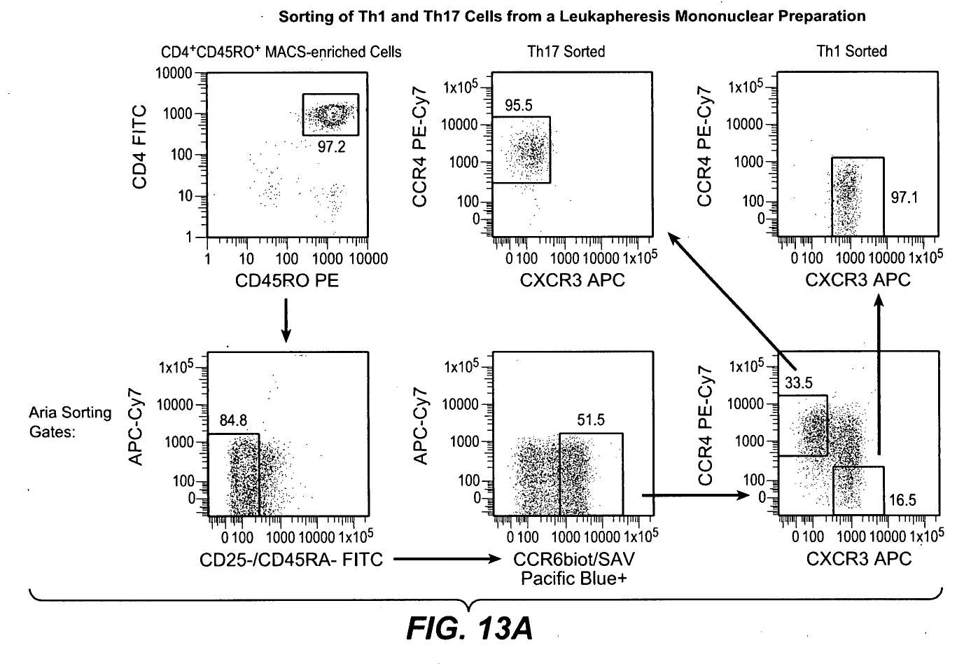

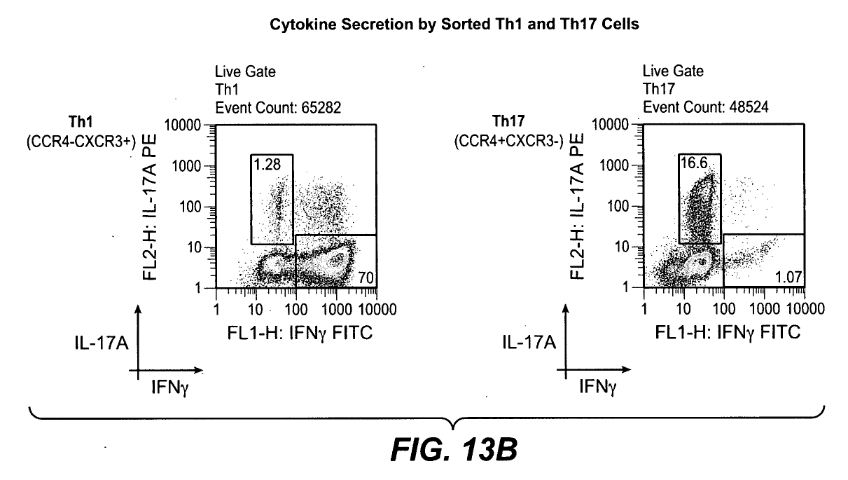

Human Th1 and Th17 responder cells were sorted from a fresh leukapheresis mononuclear preparation based on their distinct expression of chemokine receptors CXCR3 and CCR4, respectively. Prior to sorting, cells were diluted with PBS, pelleted and lysed with erythrocyte lysis buffer (Qiagen). CD4+ CD45RO+ memory T cells were isolated by negative selection (Miltenyi Biotec kit and SuperMACS XS columns, according to manufacturer's instructions). Cells were then stained with CD25 FITC, CD45RA FITC, CCR4PE-Cy7, CXCR3-APC, and CCR6-biotin (BD Biosciences) at 5 μl per million cells per antibody for 20 minutes on ice and washed. Cells were then stained with streptavidin-Pacific Blue (Invitrogen) at 1:500 dilution for 15 minutes on ice and washed. Th1 and Th17 cell populations were then sorted on three BD FACS Aria cell sorters based on the following surface expression: CD25/CD45RA FITC negative, CCR6biotin/Pacific Blue positive, CXCR3-APC positive CCR4-PE-Cy7 negative (Th1) and CXCR3-APC negative CCR4-PE-Cy7 positive (Th17). Each cell population had a minimum of 95% purity after sorting as shown in FIG. 13A.

To confirm the identity of the Th1 and Th17 cell populations by their cytokine secretion, freshly sorted cells were rested overnight and stimulated the next day with PMA (1 ng/ml) and ionomycin (1 μM) with GolgiPlug (BD Biosciences) for 5 hours. Cells were fixed and permeabilized (BD Biosciences kit) and stained for intracellular IFN-gamma-FITC (BD Biosciences, 1:100 dilution) and IL-17A-PE (eBioscience, 20 μl per sample). As expected based on the published literature concerning Th1 and Th17 cell cytokine secretion, the sorted Th1 cells produced mostly IFN-γ and minimal IL-17A as shown in FIG. 13B, left panel, while the sorted Th17 cells produced mostly IL-17A and minimal IFN-γ as shown in FIG. 13B, right panel.

For the mixed lymphocyte reaction (MLR) assay, allogeneic cells were isolated from whole blood from a different donor. Blood was diluted in equal volume with PBS and overlayed on Ficoll (GE Healthcare) to isolate peripheral mononuclear cells (PBMCs). Residual red blood cells were lysed. PBMCs were irradiated 2500 rads.

The MLR assay was set up in a 96 well flat bottom plate with either 75,000 sorted Th1 cells or 75,000 sorted Th17 cells to 225,000 irradiated PBMCs with Variant D or Control IgG antibody titrations in a total volume of 240 μl per well in triplicate. The culture medium was RPMI, 10% fetal bovine serum, 1× penicillin/streptomycin, 1× gentamicin, 1×L-glutamine, 1× sodium pyruvate, 1× non-essential amino acids, and 20 mM HEPES. After 4 days of culture, cells were pulsed with 1 μCi/well of tritiated-thymidine for 17 hours, frozen, thawed, harvested, and counted.

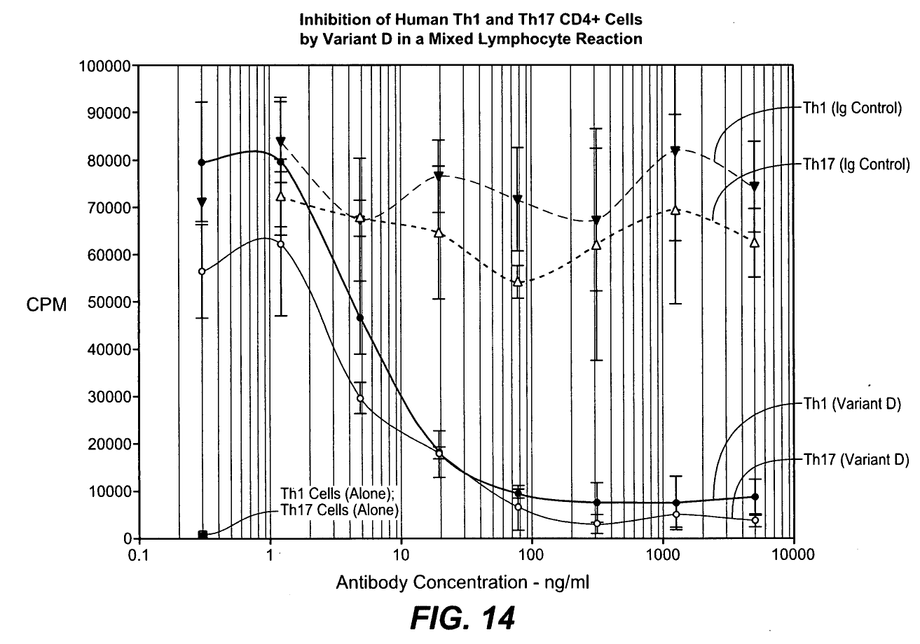

The data are shown in FIG. 14. The results show that proliferation of both Th1 and Th17 CD4+ cells was completely inhibited by Variant D in a dose-dependent manner. The Ig control failed to inhibit proliferation of either Th1 or Th17 cells (FIG. 14). Thus, these data demonstrate the potential for Variant D to attenuate the pathogenic activity of these fully differentiated subsets in the context of autoimmune disease.

To summarize, as exemplified in the Examples above, the method of the invention provides a higher therapeutic index than conventional and current therapy by minimizing toxicity and adverse side effects, including for example, but not limited to, CD4 lymphopenia and rash, and by enabling a non-intravenous method of administration, subcutaneous administration.

The foregoing written specification is considered to be sufficient to enable one skilled in the art to practice the invention. Indeed, various modifications of the invention in addition to those shown and described herein will become apparent to those skilled in the art from the foregoing description and fall within the scope of the appended claims.

1-4. (canceled)

5. A method of treating an autoimmune disease in a mammalian subject, the method comprising administering to the subject a therapeutically effective amount of a non-depleting CD4 antibody, wherein the antibody contains a modification to increase serum half-life compared to the antibody without the modification, and wherein administration of the antibody comprises a first administration and at least one subsequent administration, wherein the first administration is at a dose between 0.05 mg/kg and 35 mg/kg and each subsequent administration is at the same dose as the first administration, wherein each subsequent administration is administered between five and nine days after the previous administration, and wherein the first administration and each subsequent administration are administered subcutaneously.

6. The method of claim 5, wherein the first administration is at a dose between 1.5 mg/kg and 5.0 mg/kg.

7. The method of claim 5, wherein the antibody is an anti-human CD4 antibody and the subject is human.

8. The method of claim 5, wherein each subsequent administration is administered between six and eight days after the previous administration.

9. The method of claim 8, wherein each subsequent administration is administered seven days after the previous administration.

10. The method of claim 6, wherein the first administration is at a dose selected from 1.5 mg/kg, 2.0 mg/kg, 3.0 mg/kg, 3.5 mg/kg, and 5.0 mg/kg.

11. The method of claim 10, wherein the antibody is administered once every week.

12. The method of claim 11, wherein the antibody is administered for at least a period of time selected from one year, two years, five years, and ten years.

13. The method of claim 11, wherein the antibody is administered for the lifetime of the subject.

14. The method of claim 5, wherein the modification of the antibody increases the binding of the antibody to FcRn relative to the binding of the unmodified antibody to FcRn.

15. The method of claim 14, wherein the binding of the modified antibody to FcRn is increased between 2.0-fold and 4.5-fold relative to the binding of the unmodified antibody to FcRn.

16. The method of claim 15, wherein the binding of the modified antibody to FcRn is increased between 3.0-fold and 4.0-fold relative to the binding of the unmodified antibody to FcRn.

17. The method of claim 16, wherein the binding of the modified antibody for FcRn is increased between 3.3-fold and 3.7-fold relative to the binding of the unmodified antibody to FcRn.

18. The method of claim 17, wherein the binding of the modified antibody to FcRn is increased 3.5-fold relative to the binding of the unmodified antibody to FcRn.

19. The method of claim 5, wherein the modified antibody has reduced serum clearance compared to serum clearance of the unmodified antibody.

20. The method of claim 19, wherein the serum clearance of the modified antibody is reduced by at least 38% compared to the unmodified antibody.

21. The method of claim 20, wherein the serum clearance of the modified antibody is reduced between 38% and 59% compared to the unmodified antibody.

22. The method of claim 5, wherein the autoimmune disease is selected from lupus, systemic lupus erythematosus, cutaneous lupus erythematosus, extra renal/lupus nephritis, multiple sclerosis, relapsing-remitting multiple sclerosis, secondary-progressive multiple sclerosis, primary-progressive multiple sclerosis, rheumatoid arthritis, psoriasis, and psoriatic arthritis.

23. The method of claim 22, wherein the autoimmune disease is selected from systemic lupus erythematosus, cutaneous lupus erythematosus, and lupus nephritis.

24-25. (canceled)

26. The method of claim 5, wherein the antibody comprises the light chain CDR sequences of SEQ ID NO.: 1.

27. The method of claim 26, wherein the antibody comprises the light chain variable region sequence of SEQ ID NO.: 1.

28. The method of claim 27, wherein the antibody comprises a light chain comprising the sequence of SEQ ID NO.: 1.

29. The method of claim 5, wherein the antibody comprises the heavy chain CDR sequences of SEQ ID NO.: 6.

30. The method of claim 29, wherein the antibody comprises the heavy chain variable region sequence of SEQ ID NO.: 6.

31. The method of claim 30, wherein the antibody comprises a heavy chain comprising the sequence of SEQ ID NO.: 6.

32. The method of claim 5, wherein the antibody has a further modification that reduces binding to an Fcγ receptor as compared to the antibody without the further modification.

33. The method of claim 32, wherein the antibody comprises an Fc region that is aglycosylated.

34. The method of claim 33, wherein the antibody comprises a constant region that does not comprise a glycosylation site.

35. The method of claim 32, wherein the antibody comprises an Fc region with at least one amino acid substitution.

36. The method of claim 35, wherein the antibody comprises a N297A substitution as shown in SEQ ID NOs.: 4, 5, and 6.

37. The method of claim 36, wherein the antibody further comprises a N434A substitution as shown in SEQ ID NO.: 5.

38. The method of claim 36, wherein the antibody further comprises a N434H substitution as shown in SEQ ID NO.: 6.

39. The method of claim 28, wherein the antibody further comprises a heavy chain selected from SEQ ID NO.: 5 and SEQ ID NO.: 6.

40. The method of claim 5, wherein the antibody is a humanized antibody.

41. The method of claim 5, wherein the antibody is administered in combination with at least a second compound selected from methotrexate, leflunomide, sulfasalazine, hydroxychloroquine, a corticosteroid, and a NSAID.

42. (canceled)

43. The method of claim 5, wherein the subject previously failed at least one biologic agent.

44-52. (canceled)

53. The method of claim 5, wherein the first administration and each subsequent administration are administered subcutaneously with a self-inject device.

54. The method of claim 53, wherein the self-inject device is selected from a prefilled syringe, microneedle device, and needle-free injection device.

55-59. (canceled)

60. A method of treating an autoimmune disease in a mammalian subject, the method comprising administering to the subject a therapeutically effective amount of a non-depleting CD4 antibody, wherein the antibody contains a modification to increase serum half-life compared to the antibody without the modification, and wherein administration of the antibody comprises a first administration and at least one subsequent administration, wherein the first administration is at a flat dose between 150 mg and 350 mg and each subsequent administration is at the same dose as the first administration, wherein each subsequent administration is administered between five and nine days after the previous administration, and wherein the first administration and each subsequent administration are administered subcutaneously.

61. The method of claim 60, wherein the flat dose is between 200 mg and 300 mg.

62. The method of claim 61, wherein the flat dose is between 225 mg and 275 mg.

63. The method of claim 62, wherein the flat dose is 250 mg.

64. The method of claim 60, wherein the antibody is an anti-human CD4 antibody and the subject is human.

65. The method of claim 60, wherein each subsequent administration is administered between six and eight days after the previous administration.

66. The method of claim 60, wherein each subsequent administration is administered seven days after the previous administration.

67. The method of claim 60, wherein the antibody is administered once every week.

68. The method of claim 67, wherein the antibody is administered for at least a period of time selected from one year, two years, five years, and ten years.

69. The method of claim 67, wherein the antibody is administered for the lifetime of the subject.

70. The method of claim 60, wherein the modification of the antibody increases the binding of the antibody to FcRn relative to the binding of the unmodified antibody to FcRn.

71. The method of claim 70, wherein the binding of the modified antibody to FcRn is increased between 2.0-fold and 4.5-fold relative to the binding of the unmodified antibody to FcRn.

72. The method of claim 71, wherein the binding of the modified antibody to FcRn is increased between 3.0-fold and 4.0-fold relative to the binding of the unmodified antibody to FcRn.

73. The method of claim 72, wherein the binding of the modified antibody for FcRn is increased between 3.3-fold and 3.7-fold relative to the binding of the unmodified antibody to FcRn.

74. The method of claim 73, wherein the binding of the modified antibody to FcRn is increased 3.5-fold relative to the binding of the unmodified antibody to FcRn.

75. The method of claim 60, wherein the modified antibody has reduced serum clearance compared to serum clearance of the unmodified antibody.

76. The method of claim 75, wherein the serum clearance of the modified antibody is reduced by at least 38% compared to the unmodified antibody.

77. The method of claim 76, wherein the serum clearance of the modified antibody is reduced between 38% and 59% compared to the unmodified antibody.

78. The method of claim 60, wherein the autoimmune disease is selected from lupus, systemic lupus erythematosus, cutaneous lupus erythematosus, extra renal/lupus nephritis, multiple sclerosis, relapsing-remitting multiple sclerosis, secondary-progressive multiple sclerosis, primary-progressive multiple sclerosis, rheumatoid arthritis, psoriasis, and psoriatic arthritis.

79. The method of claim 78, wherein the autoimmune disease is selected from systemic lupus erythematosus, cutaneous lupus erythematosus, and lupus nephritis.

80-81. (canceled)

82. The method of claim 60, wherein the antibody comprises the light chain CDR sequences of SEQ ID NO.: 1.

83. The method of claim 82, wherein the antibody comprises the light chain variable region sequence of SEQ ID NO.: 1.

84. The method of claim 83, wherein the antibody comprises a light chain comprising the sequence of SEQ ID NO.: 1.

85. The method of claim 60, wherein the antibody comprises the heavy chain CDR sequences of SEQ ID NO.: 6.

86. The method of claim 85, wherein the antibody comprises the heavy chain variable region sequence of SEQ ID NO.: 6.

87. The method of claim 86, wherein the antibody comprises a heavy chain comprising the sequence of SEQ ID NO.: 6.

88. The method of claim 60, wherein the antibody has a further modification that reduces binding to an Fcγ receptor as compared to the antibody without the further modification.

89. The method of claim 88, wherein the antibody comprises an Fc region that is aglycosylated.

90. The method of claim 89, wherein the antibody comprises a constant region that does not comprise a glycosylation site.

91. The method of claim 88, wherein the antibody comprises an Fc region with at least one amino acid substitution.

92. The method of claim 91, wherein the antibody comprises a N297A substitution as shown in SEQ ID NOs.: 4, 5, and 6.

93. The method of claim 92, wherein the antibody further comprises a N434A substitution as shown in SEQ ID NO.: 5.

94. The method of claim 92, wherein the antibody further comprises a N434H substitution as shown in SEQ ID NO.: 6.

95. The method of claim 84, wherein the antibody further comprises a heavy chain selected from SEQ ID NO.: 5 and SEQ ID NO.: 6.

96. The method of claim 60, wherein the antibody is a humanized antibody.

97. The method of claim 60, wherein the antibody is administered in combination with at least a second compound selected from methotrexate, leflunomide, sulfasalazine, hydroxychloroquine, a corticosteroid, and a NSAID.

98. (canceled)

99. The method of claim 60, wherein the subject previously failed at least one biologic agent.

100-106. (canceled)

107. The method of claim 60, wherein the first administration and each subsequent administration are administered subcutaneously with a self-inject device.

108. The method of claim 107, wherein the self-inject device is selected from a prefilled syringe, microneedle device, and needle-free injection device.

109-116. (canceled)
